(19) United States
(12) Patent Application Publication
(10) Pub. No.: US 2014/0010817 A1
(43) Pub. Date: Jan. 9, 2014

(54) Novel Antibodies and Uses Thereof

(71) Applicant: Daiichi Sankyo Company, Limited, (US)

(72) Inventors: Satomichi Yoshimura, Hyogo (JP); Tatsuya Kurihara, Hyogo (JP); Kayoko Kawashima, Hyogo (JP); Masato Hoshino, Hyogo (JP); Kumiko Kadoshima, Hyogo (JP); Maki Tsujimoto, Hyogo (JP); Takako Kimura, Tokyo (JP); Jun Hasegawa, Tokyo (JP)

(73) Assignee: Daiichi Sankyo Company, Limited, Tokyo (JP)

(21) Appl. No.: 13/894,865

(22) Filed: May 15, 2013

Related U.S. Application Data
(60) Provisional application No. 61/647,992, filed on May 16, 2012.

(30) Foreign Application Priority Data
May 16, 2012 (JP) 2012-112029

(51) Int. Cl. C07K 16/10; A61K 45/06; A61K 39/42; G01N 33/68

(52) U.S. Cl. CPC C07K 16/1036; G01N 33/6893; A61K 45/06; A61K 39/42
USPC 424/139.1; 530/387.9; 530/387.3; 536/23.53; 435/320.1; 435/69.6; 435/7.94; 435/331; 435/252.33; 435/252.31; 435/254.2; 435/419

(57) ABSTRACT

The present invention provides an antibody that recognizes a polypeptide comprising the amino acid sequence represented by SEQ ID NO: 15 in the Sequence Listing and has an anti-arthritic function, or a functional fragment thereof.

### BACKGROUND OF THE INVENTION

1. Field of the Invention

The present invention relates to: an antibody that recognizes a desired antigen and has a desired activity; the antibody having particular complementarity determining region(s) (hereinafter, referred to as “CDR(s)”); a chimeric antibody, a humanized antibody, or a human antibody having these CDRs; a functional fragment of the antibody; a modified form of the antibody or the functional fragment thereof; a nucleic acid encoding the amino acid sequence of the antibody or the functional fragment thereof, or the modified form of the antibody or the functional fragment; a recombinant vector containing an insert of this nucleic acid; a recombinant cell containing this vector introduced therein; a ceil producing the antibody; a method for producing the antibody, comprising the steps of culturing any of these cells and collecting the desired antibody from the cultures; a pharmaceutical composition comprising the antibody; a pharmaceutical composition for treatment or prevention of various diseases; a method for treating or preventing various diseases, comprising the step of administering the antibody; use of the antibody for preparing a pharmaceutical composition for treatment or prevention of various diseases; use of the antibody for treating of preventing various diseases; a composition for examination or diagnosis of various diseases, comprising the antibody; a method for examining or diagnosing various diseases using the antibody; use of the antibody for preparing a composition for examination or diagnosis of various diseases; use of the antibody for examining or diagnosing various diseases; etc.

### BACKGROUND INVENTION

Rheumatoid arthritis (hereinafter, referred to as “RA”) is a chronic inflammatory disease that principally causes inflammation in the joint synovium and eventually results in joint dysfunction through bone or cartilage destruction. This serious disease significantly reduces the quality of life (QOL) of the patient. In RA, an abnormal immune system attacks the patient's own joint synovium, causing inflammation. As a result, symptoms such as joint pain, swelling, and deformity occur. The morbidity of RA is 0.5 to 1.0% of the population in a developed country and increases with aging (Non-Patent Document 1),

RA has previously been treated by therapy centered on disease-modifying antirheumatic drugs (DMARDs) such as methotrexate (MTX) or steroids. Such treatment has exhibited anti-inflammatory action to some extent, but has not been sufficiently effective for preventing joint destruction. In recent years, advanced treatment methods using anti-TNF biologics, anti-IL-6 receptor antibodies, or CTLA4-Ig have been introduced. Reportedly, these treatment methods have ameliorated RA in some patients and thus have received attention because of their high effectiveness. The introduction of such biologics is changing the treatment of RA from conservative therapy, which delays the progression of the disease, to active therapy aimed at the induction of remission.

Unfortunately, administered anti-TNF biologics are insufficiently effective for 30 to 40% of treated patients and thus cannot lead all RA patients to complete remission (Non-Patent Document 2). In addition, the mechanism underlying the pharmaceutical efficacy of steroids or conventional biologics is based on immunosuppressive action, which disadvantageously increases the risk of infection (Non-Patent Document 3). The development of safer and more effective therapeutic drugs for RA requires establishing a treatment method based on the novel mechanism of action of the cause of RA.

Although the mechanism underlying the onset of RA remains to be elucidated, genetic factors such as mutations in so-called RA-sensitive genes including HLA-DR4, PADI4, PTPN22, and TNFAIP3 have been proposed (Non-Patent Document 4). Also, environmental factors such as hormone imbalance caused by aging, stress, delivery, smoking, etc, or bacterial or viral infection are considered important. As for bacteria, the relation of mycoplasma or streptococcus to RA has been suggested, while the relation of infection with DNA viruses such as EB virus, type B hepatitis virus, herpesvirus, and parvovirus to the onset of RA has been pointed out (Non-Patent Document 5). Some retrovirus-derived factors are regarded as important factors causative of the onset of RA. HIV or HTLV-I increases the expression of oncogenes that lead synovial cells to abnormal growth. On the other hand, HIV or HTLV-I causes the expression of transcriptional activators called tax or tat, resulting in the production of inflammatory cytokines such as IL-1 or IL-6. These two mechanisms are hypothetical events to induce RA (Non-Patent Document 6). It has also been suggested that the group-specific antigen protein (gag), superantigen (sag), or envelope protein (env) of human endogenous retrovirus (HERV) is involved in the crises or RA by excessively activating T cells through its action on T cell receptors and thereby destroying immune functions (Non-Patent Document 7). In actuality, the onset of RA may arise from a complex combination of such genetic factors and environmental factors.

Murine mammary tumor virus (hereinafter, referred to as “MMTV”) is a retrovirus that induces breast cancer in mice. Reportedly, its presence in human patients with breast cancer has been suggested (Non-Patent Document 8), and infection with MMTV may influence mouse immune functions (Non-Patent Document 9). The relation of MMTV to RA, however, has not been known.

Some monoclonal antibodies against MMTV env are known to have a suppressive effect on MMTV infection or growth (Non-Patent Document 10 and 11). Nonetheless, an anti-MMTV env antibody that suppresses the onset and exacerbation of RA or arthritis has not yet been disclosed.

### PRIOR ART DOCUMENTS

### Non-Patent Documents

Non-Patent Document 1: Scott D L et al., “Lancet”, 2010, Vol. 376, p. 1094-1108

Non-Patent Document 2: Plant D et al., “Arthritis Rheumatology”, 2011, Vol. 63, No. 3, p. 645-653

Non-Patent Document 3: Tokuda H et al., “Internal Medicine”, 2008, Vol. 47, p. 915-923

Non-Patent Document 4: Dieude P., “Joint Bone Spine”, 2009, Vol. 76, No. 6, p. 602-607

Non-Patent Document 5: Berkun Y and Padeh S., “Autoimmunity Reviews”, 2010, Vol. 9, No. 5, p. A319-324

Non-Patent Document 6: Kalden J R and Gay S., “Clinical and Experimental Immunology”, 1994, Vol. 98, No. 1, p. 1-5

Non-Patent Document 7: Balada E et al., “Reviews in Medical Virology”, 2098, Vol. 19, p. 273-286

Non-Patent Document 8: Taneja P et al, “Expert Review of Molecular Diagnostics”, 2008, Vol. 9, No. 5, p. 423-440

Non-Patent Document 9: Acha-Orbea H et al. “Frontiers in Bioscience”, 2007, Vol. 12, p. 1594-1604

Non-Patent Document 10: Mpandi M J et al., “The Journal of Virology”, 2003, Vol. 77, No. 17, p. 9369-9377

Non-Patent Document 11: Indik S et al., “Cancer Research”, 2005, Vol. 65, No. 15, p. 6651-6659

An object of the present invention is to provide an antibody that recognizes the protein of the present invention and suppresses arthritis, a functional fragment thereof, or a modified form of the antibody or the functional fragment. An alternative object of the present invention is to provide a pharmaceutical composition comprising this antibody. A further alternative object of the present invention is to provide a pharmaceutical composition for treatment or prevention of autoimmune disease such as RA. A further alternative object of the present invention is to provide a composition for examination or diagnosis of the onset, exacerbation, degree of progression, therapeutic effects, etc., of autoimmune disease such as RA or for diagnosis of the disease. A further alternative object of the present invention is also to provide a method for producing the antibody of the present invention, a cell that is subjected to this production method, a recombinant vector introduced in this cell, a nucleic acid inserted in this vector, a cell producing the antibody of the present invention, etc.

### SUMMARY OF THE INVENTION

The present invention relates to, for example,

(1) An antibody that recognizes a polypeptide comprising any one of the following amino acid sequences (I) to (III) and has an anti-arthritic function, or a functional fragment thereof:

(I) the amino acid sequence represented by SEQ ID NO: 15 is in the Sequence Listing;

(II) the amino acid sequence, that is encoded by the nucleotide sequence of a nucleic acid hybridizing under stringent conditions to a nucleic acid having a nucleotide sequence complementary to a nucleotide sequence encoding the amino acid sequence represented by SEQ ID NO: 15 in the Sequence Listing, and of a polypeptide that causes the onset and/or exacerbation of arthritis; and

(III) the amino acid sequence, that comprises an amino acid sequence represented by SEQ ID NO: 15 in the Sequence Listing having the substitution, deletion, addition, or insertion of one to several amino acids, and of a polypeptide that causes the onset and/or exacerbation of arthritis;

(2) The antibody or the functional fragment thereof according to (1), wherein the polypeptide has a molecular weight of (I) 50 to 55 k, (II) 50 to 55 k and 2 5 to 30 k, or (III) 70 to 75 k under non-reducing conditions of SDS-PAGE;

(3) The antibody or the functional fragment thereof according to (1) or (2) wherein the antibody or the functional fragment thereof suppresses bone destruction;

(4) The antibody or the functional fragment thereof according to (3), wherein the bone destruction is a process in a collagen-induce arthritis non-human animal model;

The antibody or the functional fragment thereof according to any one of (1) to (4), wherein the anti-arthritic function works in a collagen-induced arthritis non-human animal model;

(6) The antibody or the functional fragment thereof according to any one of (1) to (5), wherein the polypeptide described in (1) exacerbates arthritis in a collagen-induced arthritis non-human animal model;

(7) The antibody of the functional fragment thereof described in (1) is capable of being detected in a collagen-induced arthritis mouse model;

(8) The antibody or the functional fragment thereof according to any one of (1) to (7), wherein the antibody or the functional fragment thereof inhibits cytokine production in an inflamed (body) region;

(9) The antibody or the functional fragment thereof according to (8), wherein the cytokine is an inflammatory cytokine and/or a chemokine;

(10) The antibody or the functional fragment thereof according to (8) or (9), wherein the inflamed (body) region is an affected part in a collagen-induced arthritis non-human animal model;

(11) The antibody or the functional fragment thereof according to any one of (4) to (6) and (10), wherein the non-human animal is a mouse;

(12) The antibody or the functional fragment thereof according to any one of (1) to (11), wherein the antibody or the functional fragment thereof recognizes the polypeptide comprising the amino acid sequence represented by SEQ ID NO: 15 in the Sequence Listing;

(13) The antibody or the functional fragment thereof according to any one of (1) to (12), wherein the antibody consists of a heavy chain comprising CDRH1 consisting of the amino acid sequence represented by SEQ ID NO: 22 in the Sequence Listing, CDRH2consisting of the amino acid sequence represented by SEQ ID NO: 23 in the Sequence Listing, and CDRH3 consisting of the amino acid sequence represented by SEQ ID NO: 24 in the Sequence Listing, and a light chain comprising CDRL1 consisting of the amino acid sequence represented by SEQ ID NO: 25 in the Sequence Listing, CDRL2consisting of the amino acid sequence represented by SEQ ID NO: 26 in the Sequence Listing, and CDRL3 consisting of the amino acid sequence represented by SEQ ID NO: 27 in the Sequence Listing;

(14) The antibody of the functional fragment thereof according to any one of (1) to (12), wherein the antibody consists of a heavy chain comprising CDRH1 consisting of the amino acid sequence represented by SEQ ID NO: 36 in the Sequence Listing, CDRH2 consisting of the amino acid sequence represented by SEQ ID NO: 37 in the Sequence Listing, and CDRH3 consisting of the amino acid sequence represented by SEQ ID NO: 33 in the Sequence Listing, and a light chain comprising CDRL1 consisting of the amino acid sequence represented by SEQ ID NO: 33 in the Sequence Listing, CDRL2 consisting of the amino acid sequence represented by SEQ ID NO: 40 in the Sequence Listing, and CDRL3 consisting of the amino acid sequence represented by SEQ ID NO: 41 in the Sequence Listing;

(15) The antibody or the functional fragment thereof according to any one of (1) to (12), wherein the antibody consists of a heavy chain comprising CDRH1 consisting of the amino acid sequence represented by SEQ ID NO: 66 in the Sequence Listing, CDRH2 consisting of the amino acid sequence represented by SEQ ID NO: 67 in the Sequence Listing, and CDRH3 consisting of the amino acid sequence represented by SEQ ID NO: 68 in the Sequence Listing, and a light chain comprising CDRL1 consisting of the amino acid sequence represented by SEQ ID NO: 69 in the Sequence Listing, CDRL2consisting of the amino acid sequence represented by SEQ ID NO: 70 in the Sequence Listing, and CDRL3 consisting of the amino acid sequence represented by SEQ ID NO: 71 in the Sequence Listing;

(16) The antibody or the functional fragment thereof according to any one of (1) to (12), wherein the antibody consists of a heavy chain comprising CDRH1 consisting of the amino acid sequence represented by SEQ ID NO: 112 in the Sequence Listing, CDRH2 consisting of the amino acid sequence represented by SEQ ID NO: 113 in the Sequence Listing, and CDRH3 consisting of the amino acid sequence represented by SEQ ID NO: 114 in the Sequence Listing, and a light chain comprising CDRL1 consisting of the amino acid sequence represented by SEQ ID NO: 115 in the Sequence Listing, CDRL2consisting of the amino acid sequence represented by SEQ ID NO: 116 in the Sequence Listing, and CDRL3 consisting of the amino acid sequence represented by SEQ ID NO: 117 in the Sequence Listing;

(17) The antibody or the functional fragment thereof according to any one of (1) to (12), wherein the antibody comprises heavy and light chains comprising amino acid sequences 95% or higher identical to the amino acid sequences of the heavy and light chains, respectively, of an antibody according to any one of (13) to (16) and recognizes the polypeptide according to (1);

(18) The antibody or the functional fragment thereof according to any one of (1) to (12), wherein the antibody or the functional fragment thereof binds to a site on an antigen recognized by an antibody or a functional fragment thereof according to any one of (13) to (16);

(19) The antibody or the functional fragment thereof according to any one of (1) to (12), wherein the antibody or the functional fragment thereof competes with an antibody or a functional fragment thereof according to any one of (13) to (16) for binding to the polypeptide according to (1);

(20) The antibody or the functional fragment thereof according to any one of (1) to (19), wherein the antibody is a chimeric antibody;

(21) The antibody or the functional fragment thereof according to any one of 1) to (19), wherein the antibody is a humanized antibody;

(22) The antibody or the functional fragment thereof according to any one of (1) to (19), wherein the antibody is a human antibody;

(23) Any one of the following nucleic acids (I) to (III):

(I) a nucleic acid comprising a nucleotide sequence encoding a partial or whole amino acid sequence of the heavy or light chain of an antibody according to any one of (1) to (22);

(II) a nucleic acid consisting of a nucleotide sequence comprising a nucleotide sequence encoding a partial or whole amino acid sequence of the heavy or light chain of an antibody according to any one of (1) to (22); and

(III) a nucleic acid consisting of a nucleotide sequence encoding a partial or whole amino acid sequence of the heavy or light chain of an antibody according to any one of (1) to (22);

(24) A recombinant vector containing an insert of a nucleic acid according to (23);

(25) A recombinant cell containing a nucleic acid according to (23) or a recombinant vector according to (24) introduced therein;

(26) A cell producing an antibody according to any one of (1) to (22);

(27) A method for producing an antibody or a functional fragment thereof according to any one of (1) to (22), comprising the following steps (I) and (II):

(I) culturing a cell according to (25) or (26); and

(II) collecting the antibody or the functional fragment thereof according to any one of (1) to (22) from the cultures obtained in step (I);

(28) The antibody or the functional fragment thereof according to any one of (1) to (22), wherein the antibody or the functional fragment thereof is obtained by a method according to (27):

(29) A modified form of an antibody or a functional fragment thereof according to any one of (1) to (22) and (28);

(30) A pharmaceutical composition comprising an antibody or a functional fragment thereof according to any one of (1) to (22) and (28) or a modified form according to (29) as an active ingredient;

(31) The pharmaceutical composition according to (30), wherein the pharmaceutical composition is a therapeutic or prophylactic drug for autoimmune disease in an individual expressing a polypeptide according to (1);

(32) The pharmaceutical composition according to (31), wherein the autoimmune disease is rheumatoid arthritis;

(33) The pharmaceutical composition according to (30), wherein the pharmaceutical composition is a therapeutic or preventive drug; for arthritis in an individual expressing a polypeptide according to (1);

(34) A composition for examination or diagnosis of rheumatoid arthritis, comprising an antibody or a functional fragment thereof according to any one of (1) to (22) and (28) or a modified form according to (29);

(35) The antibody or the functional fragment thereof according to (13), wherein the heavy chain variable region comprises a peptide represented by an amino acid sequence described in any one of SEQ ID NOs: 72 to 81 (FIGS. 40 to 49) in the Sequence Listing, and the light chain variable region comprises a peptide represented by an amino acid sequence described in any one of SEQ ID NOs: 82 to 86 (FIGS. 50 to 54) in the Sequence Listing;

(36) The antibody or the functional fragment thereof according to (13), wherein the heavy chain variable region is a peptide represented by an amino acid sequence described in any one of SEQ ID NOs: 72 to 81 (FIGS. 40 to 49) in the Sequence Listing, and the light chain variable region is a peptide represented by an amino acid sequence described in any one of SEQ ID NOs: 82 to 86 (FIGS. 50 to 54) in the Sequence Listing;

(37) The antibody or the functional fragment thereof according to (13), wherein the heavy chain variable region is represented by an amino acid sequence consisting of amino acid Nos. 20 to 138 of SEQ ID NO: 19 (FIG. 16) in the Sequence Listing, and the light chain variable region is represented by an amino acid sequence consisting of amino acid Nos. 21 to 128 of SEQ ID NO: 21 (FIG. 18) in the Sequence Listing;

(38) The antibody or the functional fragment thereof according to (35), wherein the heavy chain variable region comprises a peptide represented by an amino acid sequence selected from the group consisting of the amino acid sequences described in SEQ ID NOs: 72 to 74, 76, and 79 to 81(FIGS. 40 to 42, 44, and 47 to 49) in the Sequence Listing, and the light chain variable region comprises a peptide represented by the amino acid sequence of SEQ ID NO: 82 (FIG. 50) in the Sequence Listing;

(39) The antibody or the functional fragment thereof according to (36), wherein the heavy chain variable region is a peptide represented by an amino acid sequence selected from the group consisting of the amino acid sequences described in SEQ ID NOs: 72 to 74, 76, and 79 to 81 (FIGS. 40 to 42, 41, and 47 to 49) in the Sequence Listing, and the light chain variable region is a peptide represented by the amino acid sequence of SEQ ID NO: 82 (FIG. 50) in the Sequence Listing;

(40) The antibody or the functional fragment thereof according to (35), wherein the heavy chain variable region comprises a peptide represented by an amino acid sequence selected from the group consisting of the amino acid sequences described in SEQ ID NOs: 72 to 74 and 76 to 78 (FIGS. 40 to 42 and 44 to 46) in the Sequence Listing, and the light chain variable region comprises a peptide represented by the amino acid sequence of SEQ ID NO: 33 (FIG. 51) in the Sequence Listing;

(11) The antibody or the functional fragment thereof according to (36), wherein the heavy chain variable region is a peptide represented by an amino acid sequence selected from the group consisting of the amino acid sequences described in SEQ ID NOs : 72 to 74, and 76 to 78 (FIGS. 40 to 42 and 44 to 46) in the Sequence Listing, and the light chain variable region is a peptide represented by the amino acid sequence of SEQ ID NO: 83 (FIG. 31) in the Sequence Listing;

(42) The antibody or the functional fragment thereof according to (35), wherein the heavy chain variable region comprises a peptide represented by an amino acid sequence described in any one of SEQ ID NOs: 72 to 74 (FIGS. 40 to 42) in the Sequence Listing, and the light chain variable region comprises a peptide represented by an amino acid sequence of SEQ ID NO: 81 (FIG. 52) in she Sequence Listing;

(43) The antibody or the functional fragment thereof according to (36), wherein the heavy chain variable region is a peptide represented by an amino acid sequence described in any one of SEQ ID NOs: 72 to 74 (FIGS. 40 to 42) in the Sequence Listing, and the light chain variable region is a peptide represented by an amino acid sequence of SEQ ID NO: 84 (FIG. 52) in the Sequence Listing;

(44) The antibody or the functional fragment thereof according to (35), wherein the heavy chain variable region comprises a peptide represented by the amine acid sequence of SEQ ID NO: 75 (FIG. 43) in the Sequence Listing, and the light chain variable region comprises a peptide represented by the amino acid sequence of SEQ ID NO: 85 (FIG. 53) in the Sequence Listing;

(45) the antibody or the functional fragment thereof according to (36), wherein the heavy chain variable region is a peptide represented by the amino acid sequence of SEQ ID NO: 75 (FIG. 43) in the Sequence Listing, and the light chain variable region is a peptide represented by the amino acid sequence of SEQ ID NO: 85 (FIG. 53) in the Sequence Listing;

(46) The antibody or the functional fragment thereof according to (35), wherein the heavy chain variable region comprises a peptide represented by an amino acid sequence selected from the group consisting of the amino acid sequences described in SEQ ID NOs: 73, 74, 76, and 77 (FIGS. 41, 42, 44 and 45) in she Sequence Listing, and the light chain variable region comprises a peptide represented by the amino acid sequence of SEQ ID NO: 86 (FIG. 54) in the Sequence Listing;

(47) The antibody or the functional fragment thereof according to (36), wherein the heavy chain variable region is a peptide represented by an amino acid sequence selected from the group consisting of the amino acid sequences described in SEQ ID NOs : 73, 74, 76 and 77 (FIGS. 41, 42, 44 and 45) in the Sequence Listing, and the light chain variable region is a peptide represented by the amino acid sequence of SEQ ID NO: 86 (FIG. 54) in the Sequence Listing;

(48) An antibody selected from the following (i) to (xxi), or a functional fragment thereof:

(i) an antibody (T13) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(ii) an antibody (T14) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 33 (FIG. 51) in the Sequence Listing and a human IgG1-derived constant region;

(iii) an antibody (T15) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 86 (FIG. 51) in the Sequence Listing and a human IgG1-derived constant region;

(iv) an antibody (T8) that consists of a heavy chain having a variable region consisting of the amino acid sequence represented by SEQ ID NO: 74 (FIG. 47) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(v) an antibody (T9) that consists of a heavy chain having a variable region consisting of the amino acid sequence represented by SEQ ID NO: 71 (FIG. 42) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;

(vi) an antibody (T10) that consists of a heavy chain having a variable region consisting of the amino acid sequence represented by SEQ ID NO: 74 (FIG. 42) in the Sequence Listing and a human IgG1-derived constant region, and a light, chain having a variable region consisting of the amino acid sequence represented by SEQ ID NO: 84 (FIG. 52) in the Sequence Listing and a human IgG1-derived constant region;

(vii) an antibody (T11) that consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region;

(viii) an antibody (T18) that consists of a heavy chain having a variable region consisting of the amino acid sequence represented by SEQ ID NO: 78 (FIG. 46) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;

Sequence Listing and a human IgG1-derived constant region;

(ix) an antibody (T12) that consists of a heavy chain having a variable region consisting of the amino acid sequence represented by SEQ ID NO: 75 (FIG. 43) in the Sequence Listing and a human IgG1-derived constant region, and a light chain having a variable region consisting of the amino acid sequence represented by SEQ ID NO: 85 (FIG. 53) in the Sequence Listing and a human IgG1-derived constant region;

(x) an antibody (T1) that consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(xi) an antibody (T2) that consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;

(xii) an antibody (T3) that consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region;

(xiii) an antibody (T4) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(xiv) an antibody (T5) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;

(xv) an antibody (T6) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region;

(xvi) an antibody (T7) that consists of a heavy chain having a variable region consisting of the amino acid sequence represented by SEQ ID NQ: 73 (FIG. 41) in the Sequence Listing and a human IgG1-derived constant region, and a light chain having a variable region consisting of the amino acid sequence represented by SEQ ID NO: 36 (FIG. 54) in the Sequence Listing and a human IgG1-derived constant region;

(xvii) an antibody (T16) that consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 63 (FIG. 51) in the Sequence Listing and a human IgG1-derived constant region;

(xviii) an antibody (T17) chat consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region;

(xix) an antibody (T19) that consists of a heavy chain having a variable region consisting of the amino acid sequence represented by SEQ ID NO: 79 (FIG. 47) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(xx) an antibody (T20) that consists of a heavy chain having a variable region consisting of the amino acid sequence represented by SEQ ID NO: 80 (FIG. 48) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region; and

(xxi) an antibody (T21) that consists of a heavy chain having a variable region consisting of the amino acid sequence represented by SEQ ID NO: 81 (FIG. 49) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;

(49) The antibody or the functional fragment thereof according to any one of (1) to (22) and (28), wherein the antibody comprises heavy and light chains comprising amino acid sequences 95% or higher identical to the amino acid sequences of the heavy and light chains, respectively, of an antibody according to any one of (35) to (48) and recognizes the polypeptide described in (1);

(50) The antibody or the functional fragment thereof according to any one of (1) to (22) and (28), wherein the antibody or the functional fragment thereof binds to a site on an antigen recognized by an antibody or a functional fragment thereof according to any one of (35) to (48);

(51) The antibody or one functional fragment thereof according to any one of (1) to (22) and (28), wherein the antibody or the functional fragment thereof competes with an antibody or a functional fragment thereof according to any one of (35) to (48) for binding to the polypeptide described in (1);

(52) Any one of the following nucleic acids (I) to (III):

(I) a nucleic acid comprising a nucleotide sequence encoding a partial or whole amino acid sequence of the heavy or light chain of an antibody according to any one of (35) to (51);

(II) a nucleic acid consisting of a nucleotide sequence comprising a nucleotide sequence encoding a partial or whole amino acid sequence of the heavy or light chain of an antibody according to any one of (35) to (51); and

(III) a nucleic acid consisting of a nucleotide sequence encoding a partial or whole amino acid sequence of the heavy or light chain of an antibody according to any one of (35) to (51);

(53) The nucleic acid according to (52), wherein the nucleotide sequence encoding a partial or whole amino acid sequence of the heavy chain of an antibody according to any one of (35) to (51) is a nucleotide sequence represented by any one of SEQ ID NOs: 91 to 100 (FIGS. 58 to 67) in the Sequence Listing, and the nucleotide sequence encoding a partial or whole amino acid sequence of the light chain of an antibody according to any one of (35) to (51) is a nucleotide sequence represented by any one of SEQ ID NOs: 103 to 107 (FIGS. 69 to 73) in the Sequence Listing;

(54) A recombinant vector containing an insert of a nucleic acid according to (52) or (53);

(55) A recombinant cell containing a nucleic acid according to (52) or (53) or a recombinant vector according to (54) introduced therein;

(56) A cell producing an antibody according to any one of (35) to (51);

(57) A method for producing an antibody or a functional fragment thereof according to any one of (35) to (51), comprising the following steps (I) and (II):

(I) culturing a cell according to (55) or (56); and

(II) collecting the antibody or the functional fragment thereof according to any one of (35) to (51) from the cultures obtained in step (I);

(58) The antibody or the functional fragment thereof according to any one of (35) to (51), wherein the antibody or the functional fragment thereof is obtained by a method according to (57);

(59) A modified form of an antibody or a functional fragment thereof according to any one of (35) to (51) and (58);

(60) A pharmaceutical composition comprising an antibody or a functional fragment thereof according to any one of (35) to (51) and (58) or a modified form according to (59) as an active ingredient;

(61) The pharmaceutical composition according to (60), wherein the pharmaceutical composition is a therapeutic or prophylactic drug for autoimmune disease in an individual expressing a polypeptide described in (1);

(62) The pharmaceutical composition according to (61), wherein the autoimmune disease is rheumatoid arthritis;

(63) The pharmaceutical composition according to (60), wherein the pharmaceutical composition is a therapeutic or prophylactic drug for arthritis in an individual expressing a polypeptide described in (1);

(61) A method for detecting a polypeptide described in (1), comprising the step of contacting a test sample with an antibody that recognizes a polypeptide comprising the amino acid sequence represented by SEQ ID NO: 15 in the Sequence Listing, or a functional fragment thereof, or a modified form of the antibody or the functional fragment;

(65) The detection method according to (64), wherein the test sample is a test subject-derived sample;

(66) The detection method according to (65), wherein the test subject-derived sample is plasma;

(67) A method for quantifying RX protein, comprising the step of contacting a test sample with an antibody that recognizes a polypeptide comprising the amino acid sequence represented by SEQ ID NO: 15 in the Sequence Listing, or a functional fragment thereof, or a modified form of the antibody or the functional fragment;

(68) The quantification method according to (67), wherein the test sample is a test subject-derived sample;

(69) The quantification method according to (68), wherein the test subject-derived sample is plasma;

(70) The method according to any one of (64) to (69), wherein the method is performed using an antibody that recognizes a polypeptide comprising the amino acid sequence represented by SEQ ID NO: 15 in the Sequence Listing, or a functional fragment thereof, or a modified form of the antibody or the functional fragment;

(71) The method according to any one of (64) to (69), wherein the method is performed using two or more antibodies that each recognize a polypeptide comprising the amino acid sequence represented by SEQ ID NO: 15 in the Sequence Listing, functional fragments thereof, or modified forms of the antibodies or the functional fragments;

(72) The method according to (71), wherein the method is performed using sandwich ELISA;

(73) A method for examining rheumatoid arthritis, comprising the step of contacting a test sample with an antibody that recognizes a polypeptide comprising the amino acid sequence represented by SEQ ID NO: 15 in the Sequence Listing, or a functional fragment thereof, or a modified form of the antibody or the functional fragment;

(74) The examination method according to (73), wherein the test sample is a test subject-derived sample;

(75) The examination method according to (74), wherein the test subject-derived sample is plasma;

(76) A method for diagnosing rheumatoid arthritis, comprising the following steps (I) to (III):

(I) contacting test subject-derived plasma with an antibody that recognizes a polypeptide comprising the amino acid sequence represented by SEQ ID NO: 15 in the Sequence Listing, or a functional fragment thereof, or a modified form of the antibody or the functional fragment;

(II) determining the amount of a polypeptide described in (1) in the test subject-derived plasma; and

(III) diagnosing the test subject as having rheumatoid arthritis or as being at a high risk of developing rheumatoid arthritis when the amount of the polypeptide described in step (II) in the test subject-derived plasma is greater than that in healthy individual-derived plasma;

(77) A composition for assay of a polypeptide comprising the amino acid sequence represented by SEQ ID NO: 15 in the Sequence Listing or for diagnosis, comprising an antibody that recognizes the polypeptide, or a functional fragment thereof, or a modified form of the antibody or the functional fragment;

(38) The composition according to (77), wherein the diagnosis is diagnosis of rheumatoid arthritis:

(79) The composition according to (77) or (78), wherein the composition comprises an antibody that recognizes a polypeptide comprising the amino acid sequence represented by SEQ ID NO: 15 in the Sequence Listing, or a functional fragment thereof, or a modified form of the antibody or the functional fragment;

(80) The composition according to (77) or (78), wherein the composition comprises two or more antibodies that each recognize a polypeptide comprising the amino acid sequence represented by SEQ ID NO: 15 in the Sequence Listing, functional fragments thereof, or modified forms of the antibodies or the functional fragments;

(81) The composition according to (80), wherein the composition is used in sandwich ELISA;

(82) A reagent or a bit for examination or diagnosis, comprising an antibody that recognizes a polypeptide comprising the amino acid sequence represented by SEQ ID NO: 15 in the Sequence Listing, or a functional fragment thereof, or a modified form of the antibody or the functional fragment;

(83) The reagent or the kit according to (82), wherein the reagent or the kit is used in the examination or diagnosis of autoimmune disease;

(84) The reagent or the kit according to (83), wherein the autoimmune disease is rheumatoid arthritis;

(85) The reagent or the kit according to any one of (82) to (84), wherein the reagent or the kit comprises an antibody that recognizes a polypeptide comprising the amino acid sequence represented by SEQ ID NO: 15 in the Sequence Listing, or a functional fragment thereof, or a modified form of the antibody or the functional fragment;

(86) The reagent or the kit according to any one of (82) to (84), wherein the reagent or the kit comprises two or more antibodies that each recognize a polypeptide comprising the amino acid sequence represented by SEQ ID NO: 15 in the Sequence Listing, or functional fragments thereof, or modified forms of the antibodies or the functional fragments;

(87) The reagent or the kit according to any one of (82) to (86), wherein the reagent or the kit comprises a polypeptide described in (1) or a fragment thereof, or a modified form of the polypeptide or the fragment;

(88) The antibody or the functional fragment thereof according to (13), wherein the antibody is a rat antibody;

(89) The antibody or the functional fragment thereof according to any one of (14) to (10), wherein the antibody is a mouse antibody; and

(90) The pharmaceutical composition according to any one of (30) to (33) and (60) to (63), wherein the pharmaceutical composition is used in combination with an additional therapeutic or prophylactic agent.

### Effect of the Invention

The antibody provided by the present invention achieves the treatment or prevention of autoimmune disease such as RA or arthritis and the examination or diagnosis of RA or the like.

### BRIEF DESCRIPTION OF THE DRAWINGS

FIG. 1 shows the exacerbation of arthritis caused by the administration of ADSF cells to a collagen-induced arthritis animal model. The ordinate denotes an arthritis score. The abscissa denotes the number of days after initial sensitization with collagen;

FIG. 2 shows the ability of a monoclonal antibody prepared in the present invention to bind to RX protein. The ordinate denotes the amount of each antibody bound to the RX protein, wherein the amount is indicated as a relative value by absorbance;

FIG. 3 shows results of SDS-PAGE analysis of the RX protein purified from the culture supernatant of ADSF cells (sliver staining). The term “MW Marker” represents a molecular weight marker;

FIG. 4 shows results of Western blot analysis of the RX protein purified from the culture supernatant of ADSF cells, wherein Monoclonal Antibody 1 (MAb1) is used in the analysis. The term “MW Marker” denotes a molecular weight marker;

FIG. 5 shows results of MALDI-TOF-mass spectrometry (hereinafter, referred to as “MS analysis”) of the RX protein purified from the culture supernatant of ADSF cells. The term “*C” represents carbamidomethyl cysteine. The term “Position” represents a corresponding amino acid number in the amino acid sequence represented by SEQ ID NO: 15 in the Sequence Listing. The terms “Theoretical M.W.” and “Determined M.W.” represent theoretical and measured values, respectively, of a molecular weight;

FIG. 6 shows the binding affinity of the monoclonal antibody of the present invention for the RX protein. ka represents an association rate constant. kd represents a dissociation rate constant. KD represents a dissociation constant;

FIG. 7 shows the exacerbation of arthritis caused by the administration of the RX protein to a DBA/1 mouse. The ordinate denotes an arthritis score. The abscises denotes the number of days after initial sensitization with collagen. Vehicle represents a PBS-administered group used as a control;

FIG. 8 shows the suppressive action of Monoclonal Antibody 1 (MAb1) and Monoclonal Antibody 2 (MAb2) on the exacerbation of arthritis in a collagen-induced arthritis mouse model. The ordinate denotes an arthritis score. The abscissa denotes the number of days after initial sensitization with collagen;

FIG. 9 shows the suppressive action of MAb1 on bone destruction in a collagen-induced arthritis mouse model. The ordinate denotes a bone destruction score;

FIG. 10 shows results of Western blot analysis of the RX protein in the joint synovium of an RA patient. The abscissa denotes a patient number. M represents a molecular weight marker. S represents ADSF cell-derived RX protein;

FIG. 11 shows results of MALDI-TOF-MS analysis of bands of proteins purified from the plasma of an RA patient, wherein the proteins were recognized by MAb1. The term “*C” represents carbamidomethyl cysteine. The term “Position” represents a corresponding amino acid number in the amino acid sequence represented by SEQ ID NO: 15 in the Sequence Listing. The terms “Theoretical M.W.” and “Determined M.W.” represent theoretical and measured values, respectively, of a molecular weight;

FIG. 12 shows a partial amino acid sequence (which corresponds to amino acid Nos. 134 to 233 of SEQ ID NO: 15 in the Sequence Listing) translated from the nucleotide sequence of each RX gene derived from the joint synovium of an RA patient. ADSF represents an ADSF cell-derived partial sequence. Nos. 1 to 4 represent partial sequences derived from RA patients RA5 to RA8, respectively;

FIG. 13 shows the calibration curve of the RX protein obtained by sandwich ELISA (determination coefficient: R²=0.993);

FIG. 14 shows the amount of the RX protein in the plasmas of a normal subject and an RA patient. RA9 to RA26 represent samples derived from RA patients RA9 to RA26, respectively, HD1 to HD8 represent samples derived from healthy human volunteers HD1 to HD8, respectively. The ordinate denotes the concentration of the RX protein in blood;

FIG. 15 shows the nucleotide sequence (SEQ ID NO: 18 in the Sequence Listing) of an MAb1 heavy chain gene. A variable region is encoded by the nucleotides Nos. 58 to 414therein;

FIG. 16 shows the amino acid sequence (SEQ ID NO: 19 in the Sequence Listing) of the MAb1 heavy chain. The variable region is represented by amino acid Nos. 20 to 138 therein;

FIG. 17 shows the nucleotide sequence (SEQ ID NO: 20 in the Sequence Listing) of an MAb1 light chain gene. A variable region is encoded by the nucleotides Nos. 61 to 387 therein;

FIG. 18 shows the amino acid sequence (SEQ ID NO: 21 in the Sequence Listing) of the MAb1 light chain. The variable region is represented by amino acid Nos. 21 to 129 therein;

FIG. 19 shows the nucleotide sequence (SEQ ID NO: 32 in the Sequence Listing) of an MAb2 heavy chain variable region gene;

FIG. 20 shows the amino acid sequence (SEQ ID NO: 33 in the Sequence Listing) of the MAb2 heavy chain variable region;

FIG. 21 shows the nucleotide sequence (SEQ ID NO: 34 in the Sequence Listing) of an MAb2 light chain variable region gene;

FIG. 22 shows the amino acid sequence (SEQ ID NO: 39 in the Sequence Listing) of the MAb2 light chain variable region;

FIG. 23 shows amino acid sequences (SEQ ID NOs: 22 to 27, 36 to 41, and 66 to 71 in the Sequence Listing) of CDRs in the heavy and light chain variable regions of MAb1, MAb2, and Monoclonal Antibody 3 (MAb3) , respectively;

FIG. 29 shows the nucleotide sequence (SEQ ID NO: 14 in the Sequence Listing) of a gene (RX gene) encoding the amino acid sequence of ADSF ceil-derived RX protein (gp73ED), A portion (the nucleotides Nos. 1 to 294 of SEQ ID NO: 60) corresponding to a signal sequence and 3′-terminal 111 bases (except for the stop codon; the nucleotides Nos. 1954 to 2064 in the nucleotide sequence of SEQ ID NO; 60) were deleted from the nucleotide sequence of SEQ ID NO: 60 (FIG. 28);

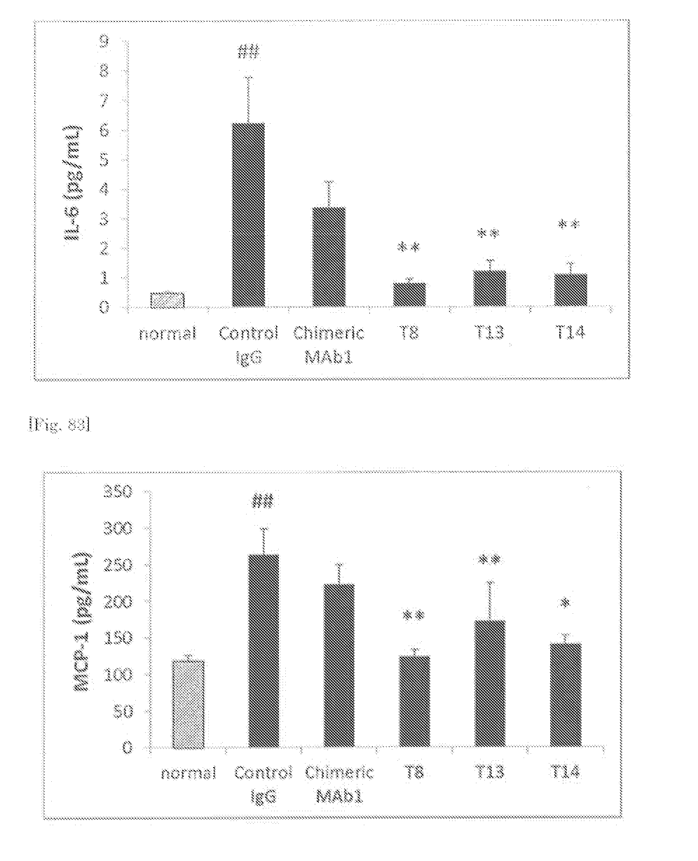

FIG. 25 shows the amino acid sequence (SEQ ID NO: 15 in the Sequence Listing) of the ADSF cell-derived RX protein gp73ED. A signal sequence (amino acid Nos. 1 to 98 of SEQ ID NO: 61) and C-terminal 37 amino acids (amino acid Nos. 652 to 688 of SEQ ID NO: 61) were deleted from the amino acid sequence of SEQ ID NO: 61 (FIG. 29);

FIG. 26 shows a nucleotide sequence (which corresponds to the nucleotides Nos. 1 to 1128 of SEQ ID NO: 14 (FIG. 22) in the Sequence Listing) encoding the amino acid sequence of gp52SU, in the ADSF cell-derived RX gene;

FIG. 27 shows the amino acid sequence (which corresponds to amino acid Nos. 1 to 376 of SEQ ID NO: 15 (FIG. 26) in the Sequence Listing) of gp52SU, in the ADSF cell-derived RX protein;

FIG. 28 shows the nucleotide sequence (SEQ ID NO: 60 in the Sequence Listing) of a gene encoding the amino acid sequence of an ADSF cell-derived RX protein precursor containing a signal sequence and a C-terminal sequence;

FIG. 29 shows the amino acid sequence (SEQ ID NO: 61 in the Sequence Listing) of the ADSF cell-derived RX protein precursor containing a signal sequence and a C-terminal sequence;

FIG. 30 shows the nucleotide sequence (SEQ ID NO: 62 in the Sequence Listing) of an MAb3 heavy chain variable region gene;

FIG. 31 shows the amino acid sequence (SEQ ID NO: 63 in the Sequence Listing) of the MAb3 heavy chain variable region;

FIG. 32 shows the nucleotide sequence (SEQ ID NO: 64 in the Sequence Listing) of an MAb3 light chain variable region gene;

FIG. 33 shows the amino acid sequence (SEQ ID NO: 65 in the Sequence Listing) of the MAb3 light chain variable region;

FIG. 34 shows the binding affinity of chimerized MAb1 for the RX protein;

FIG. 35 shows combinations of expression vectors for heavy and light chain proteins (H1 to H10 and L1 to L5, respectively) of humanized MAb1, and a number (Reference: T1 to T21) assigned to humanized MAb1 having each indicated combination;

FIG. 36 shows the binding affinity of humanized MAb1 for the RX protein;

FIG. 37 shows the preparation of an expression vector for a humanized antibody heavy chain protein;

FIG. 38 shows a calibration curve for the relative amount of the RX protein measured by ELISA using MAb2 (determination coefficient: R²=0.995);

FIG. 39 shows a calibration curve for the relative amount of the RX protein measured by ELISA using MAb3 (determination coefficient: R²=0.9961);

FIG. 40 shows the amino acid sequence (SEQ ID NO: 72 in the Sequence Listing) of the variable region of the humanized MAb1 heavy chain H1;

FIG. 41 shows the amino acid sequence (SEQ ID NO: 73 in the Sequence Listing) of the variable region of the humanized MAb1 heavy chain H2;

FIG. 42 shows the amino acid sequence (SEQ ID DO: 74 in the Sequence Listing) of the variable region of the humanized MAb1 heavy chain H3;

FIG. 43 shows the amino acid sequence (SEQ ID NO: 77 in the Sequence Listing) of the variable region of the humanized MAb1 heavy chain H4;

FIG. 44 shows the amino acid sequence (SEQ ID NO: 76 in the Sequence Listing) of the variable region of the humanized MAb1 heavy chain H5;

FIG. 45 shows the amino acid sequence (SEQ ID NO: 77 in the Sequence Listing) of the variable region of the humanized MAb1 heavy chain H6;

FIG. 46 shows the amino acid sequence (SEQ in NO; 78 in the Sequence Listing) of the variable region of the humanized MAb1 heavy chain H7;

FIG. 47 shows the amino acid sequence (SEQ ID NO: 79 in the Sequence Listing) of the variable region of the humanized MAb1 heavy chain H8;

FIG. 48 shows the amino acid sequence (SEQ ID NO: 80 in the Sequence Listing) of the variable region of the humanized MAb1 heavy chain H9;

FIG. 49 shows the amino acid sequence (SEQ ID NO: 81 in the Sequence Listing) of the variable region of the humanized MAb1 heavy chain H10;

FIG. 50 shows the amino acid sequence (SEQ ID NO: 82 in the Sequence Listing; of the variable region of the humanized MAb1 light chain L1;

FIG. 51 shows the amino acid sequence (SEQ ID NO: 83 in the Sequence Listing) of the variable region of the humanized MAb1 light chain L2;

FIG. 52 shows the amino acid sequence (SEQ ID NO: 84 in the Sequence Listing) of the variable region of the humanized MAb1 light chain L3;

FIG. 53 shows the amino acid sequence (SEQ ID NO: 85 in the Sequence Listing) of the variable region of the humanized MAb1 light chain L4;

FIG. 54 shows the amino acid sequence (SEQ ID NO: 86 in the Sequence Lasting) of the variable region of the humanized MAb1 light chain L5;

FIG. 55 shows she nucleotide sequence (SEQ ID NO: 87 in the Sequence Listing) of cDNA encoding the amino acid sequence of the heavy chain constant region of human IgG1;

FIG. 56 shows the nucleotide sequence (SEQ ID NO: 89 in the Sequence Listing) of primer F for amplification of cDNA encoding the human IgG1 heavy chain constant region;

FIG. 57 shows the nucleotide sequence (SEQ ID NO: 90 in the Sequence Listing) of primer F for amplification of cDNA encoding the human IgG1 heavy chain constant region;

FIG. 58 shows the nucleotide sequence (SEQ ID NO: 91 in the Sequence Listing) of cDNA encoding the amino acid sequence of the variable region of the humanized MAb1 heavy chain H1;

FIG. 59 shows the nucleotide sequence (SEQ ID NO: 92 in the Sequence Listing) of cDNA encoding the amino acid sequence of the variable region of the humanized MAb1 heavy chain H2;

FIG. 60 shows the nucleotide sequence (SEQ ID NO: 93 in the Sequence Listing) of cDNA encoding the amino acid sequence of the variable region of the humanized MAb1 heavy chain H3;

FIG. 61 shows the nucleotide sequence (SEQ ID NO: 94 in the Sequence Listing) of cDNA encoding the amino acid sequence of the variable region of the humanized MAb1 heavy chain H4;

FIG. 62 shows the nucleotide sequence (SEQ ID NO: 95 in the Sequence Listing) of cDNA encoding the amino acid sequence of the variable region of the humanized MAb1 heavy chain H5;

FIG. 63 shows the nucleotide sequence (SEQ ID NO: 96 in the Sequence Listing) of cDNA encoding the amino acid sequence of the variable region of the humanized MAb1 heavy chain H6;

FIG. 64 shows the nucleotide sequence (SEQ ID NO: 97 in the Sequence Listing) of cDNA encoding the amino acid sequence of the variable region of the humanized MAb1 heavy chain H7;

FIG. 65 shows the nucleotide sequence (SEQ ID NO: 98 in the Sequence Listing) of cDNA encoding the amino acid sequence of the variable region of the humanized MAb1 heavy chain H8;

FIG. 66 shows the nucleotide sequence (SEQ ID NO: 99 in the Sequence Listing) of cDNA encoding the amino acid sequence of the variable region of the humanized MAb1 heavy chain H9;

FIG. 67 shows the nucleotide sequence (SEQ ID NO: 100 in the Sequence Listing) of cDNA encoding the amino acid sequence of the variable region of the humanized MAb1 heavy chain H10;

FIG. 68 shows the nucleotide sequence (SEQ ID NO: 101 in the Sequence Listing) of the cDNA encoding the amino acid sequence of the human IgG1 light chain constant region;

FIG. 69 shows the nucleotide sequence (SEQ ID NO: 103 in the Sequence Listing) of cDNA encoding the amino acid sequence of the variable region of the humanized MAb1 light chain L1;

FIG. 70 shows the nucleotide sequence (SEQ ID NO: 104 in the Sequence Listing) of cDNA encoding the amino acid sequence of the variable region of the humanized MAb1 light chain L2;

FIG. 71 shows the nucleotide sequence (SEQ ID NO: 105 in the Sequence Listing) of cDNA encoding the amino acid sequence of the variable region of the humanized MAb1 light chain L3;

FIG. 72 shows the nucleotide sequence (SEQ ID MO; 106 in the Sequence Listing) of cDNA encoding the amino acid sequence of the variable region of the humanized MAb1 light chain L4;

FIG. 73 shows the nucleotide sequence (SEQ ID NO: 107 in the Sequence Listing) of cDNA encoding the amino acid sequence of the variable region oft he humanized MAb1 light chain L5;

FIG. 74 shows the suppressive action of humanized MAb1 on the exacerbation of arthritis in a collagen-induced arthritis mouse model. The ordinate denotes an arthritis score;

FIG. 75 shows the nucleotide sequence (SEQ ID NO: 108 in the Sequence Listing) of an MAb4 heavy chain variable region gene;

FIG. 76 shows the amino acid sequence (SEQ ID NO: 109 in the Sequence Listing) of the MAb4 heavy chain variable region;

FIG. 77 shows the nucleotide sequence (SEQ ID NO: 110 in the Sequence Listing) of an MAb4 light chain variable region gene;

FIG. 78 shows the amino acid sequence (SEQ ID NO: 111 in the Sequence Listing) of the MAb4 heavy light variable region;

FIG. 79 shows the amino acid sequences (SEQ ID NOs: 112 to 117 in the Sequence Listing) of CDRs in the heavy and light chain variable regions of MAb4;

FIG. 80 shows the suppressive action of Monoclonal Antibody 4 (MAb4) on the exacerbation of arthritis in a collagen-induced arthritis mouse model. The ordinate denotes an arthritis score. The abscissa denotes the number of days after initial sensitization with collagen;

FIG. 81 shows a calibration curve for the relative amount of the RX protein measured by ELISA using MAb4 (determination coefficient: R²=0.9905);

FIG. 82 shows the cytokine production inhibitory function of the humanized MAb1 of the present invention administered to a collagen-induced arthritis mouse model. The ordinate denotes the concentration of interleukin-6 (IL-6) in a homogenate sample derived from limbs; and

FIG. 83 shows the chemokine production inhibitory function of the humanized MAb1 of the present invention administered to a collagen-induced arthritis mouse model. The ordinate denotes the concentration of monocyte chemoattractant protein-1 (MCP-1) in a homogenate sample derived from limbs.

### DETAILED DESCRIPTION OF THE PREFERRED EMBODIMENTS

### 1. Definitions

In the present invention, the term “gene” means nucleosides (nucleic acid) comprising a nucleotide sequence encoding the amino acids of a protein, or its complementary strand. “Gene” is meant to include, for example, a polynucleotide, an oligonucleotide, DNA, mRNA, cDNA, and cRNA as the nucleotides (nucleic acid) comprising a nucleotide sequence encoding the amino acids of a protein, or its complementary strand. Such a gene is a single-stranded, double-stranded, or triple or more stranded nucleotides (nucleic acid). “Gene” is also meant to include an association of DNA and RNA strands, a mixture of ribonucleotides (RNAs) and deoxyribonucleotides (DNAs) on one nucleotides (nucleic acid) strand, and a double-stranded or triple or more stranded nucleotides (nucleic acid) comprising such a nucleotides (nucleic acid) strand. Examples of the “RX gene” of the present invention can include DNA, mRNA, cDNA, and cRNA comprising a nucleotide sequence encoding the amino acid sequence of the RX protein.

In the present invention, the term “nucleotide(s)” has the same meaning as in a “nucleic acid” and is also meant to include, for example, DNA, RNA, a probe, an oligonucleotide, a polynucleotide, and a primer. Such a nucleotide(s) is a single-stranded, double-stranded, or triple or more stranded nucleotide (nucleic acid). “Nucleotide” is also meant to include an association of DNA and RNA strands, a mixture of ribonucleotides (RNAs) and deoxyribonucleotides (DNAs) on one nucleotide (nucleic acid) strand, and an associate of two strands or three or more strands comprising such a nucleotide(s) (nucleic acid) strand.

In the present invention, the terms “polypeptide”, “peptide”, and “protein” have the same meaning.

In the present invention, the term “antigen” has the same meaning as “immunogen”.

In the present invention, the terms “RX” and “RX protein” both mean a polypeptide that comprises at least a portion of the amino acid sequence of murine mammary tumor virus envelope protein thereinafter, referred to as “MMTV env”) and causes the onset and/or exacerbation of arthritis.

In the present invention, the phrase “cause the onset and/or exacerbation or arthritis” means that a molecule directly or indirectly causes the onset and/or exacerbation or arthritis by itself, in collaboration with another factor, or in association with another factor.

In the present invention, the term “cell” also includes, for example, various cells derived from individual animals, primary cultured cells, subcultured cells, cell lines, recombinant cells, and microbial cells.

In the present invention, an antibody recognizing the RX protein is also referred to as an “anti-RX antibody”. The “anti-RX antibody” includes an anti-RX chimeric antibody, an anti-RX humanized antibody, an anti-RX human antibody, and the like.

In the present invention, the term “functional fragment of the antibody” means an antibody fragment that exerts at least a portion of functions exerted by the original antibody. Examples of the “functional fragment of the antibody” can include, but are not limited to, Fab, F(ab′)2, scFv, Fab′, and single chain immunoglobulin. Such a functional fragment of the antibody may be obtained by treating a full-length molecule of the antibody protein with an enzyme such as papain or pepsin or may be a recombinant protein produced in an appropriate host cell using a recombinant gene.

In the present invention, the “site” to which an antibody binds, i.e., the “site” recognized by an antibody, means a partial peptide or partial conformation on an antigen bound or recognized by the antibody. In the present invention, such a site is also referred to as an epitope or an antibody binding site. Examples of the site on the RX protein bound or recognized by the anti-RX antibody of the present invention can include a partial peptide or partial conformation on the RX protein.

The heavy and light chains of an antibody molecule are known to each have three complementarity determining regions (CDRs). The complementarity determining regions are also called hypervariable domains. These regions are located in the variable regions of the antibody heavy and light chains. These sites have a particularly highly variable primary structure and are usually separated at three positions on the respective primary structures of heavy and light chain polypeptide strands. In the present invention, the complementarity determining regions of the antibody are referred to as CDRH1, CDRH2, and CDRH3 from the amino terminus of the heavy chain amino acid sequence for the complementarity determining regions of the heavy chain and as CDRL1, CDRL2, and CDRL3 from the amino terminus of the light chain amino acid sequence for the complementarity determining regions of the light chain. These sites are proximal to each other on the three-dimensional structure and determine specificity for the antigen to be bound.

In the present invention, the term “antibody mutant” means a polypeptide that has an amino acid sequence derived from the amino acid sequence of the original antibody by the substitution, deletion, addition, and/or insertion (hereinafter, collectively referred to as a “mutation”) of amino acid(s) and binds to the RX protein of the present invention. The number of mutated amino acids in such an antibody mutant is 1 to 2, 1 to 3, 1 to 4, 1 to 5, 1 to 6, 1 to 7, 1 to 8, 1 to 9, 1 to 10, 1 to 12, 1 to 15, 1 to 20, 1 to 25, 1 to 30, 1 to 40, or 1 to 50. Such an antibody mutant is also encompassed by the “antibody” of the present invention.

In the present invention, the term “several” in “1 to several” refers to 2 to 10. Specifically, the term “1 to several” refers to 1 to 2, 1 to 3, 1 to 4, 1 to 5, 1 to 6, 1 to 7, 1 to 8, 1 to 9, or 1 to 10, preferably 1 to 8, more preferably 1 to 5, even more preferably 1 to 3, most preferably 1 to 2.

Examples of activities or properties exerted by the antibody of the present invention can include biological activities or physiocochemical properties and can specifically include various biological activities, a binding activity against an antigen or an epitope, stability during production or storage, and thermal stability.

In the present invention, the phrase “hybridizing under stringent conditions” means hybridization under conditions involving hybridization at 65° C. in a solution containing 5×SSC, followed by washing at 65° C. for 20 minutes in an aqueous solution containing 2×SSC−0.1% SDS, at 65° C. for 20 minuses in an aqueous solution containing 0.5×SSC−0.1% SDS, and at 65° C. for 20 minutes in an aqueous solution containing 0.2×SSC−0.1% SDS, or hybridization under conditions equivalent thereto. SSC means an aqueous solution of 150 ad NaCl−15 mM sodium citrate, and n×SSC means SSC with an n-fold concentration.

In the present invention, the term “anti-arthritic function” means the activity or function of suppressing the onset and/or exacerbation of arthritis and has the same meaning as in an “anti-arthritic activity”, an “arthritic suppressive function”, or an “arthritis suppressive activity”.

### 2. Protein

### (2-1) Properties

The RX protein of the present invention has the following properties:

(i) the RX protein has a molecular weight of 73 k (i.e., 70 to 75 k; hereinafter, a peptide that exhibits this molecular weight is referred to as “gp73ED”), approximately 55 k (i.e., 50 to 55 k; hereinafter, a peptide that exhibits this molecular weight is referred to as “gp52SU”) and approximately 28 k (i.e., 25 to 30 k; hereinafter, a peptide that exhibits this molecular weight is referred to as “qp28ED”), or approximately 55 kDa (gp52SU) only. In the present invention, the term “gp73ED” means the extracellular domain of gp73, which is full-length MMTV env. The term “gp52SU” means a domain consisting of approximately 376 amino acids counted from the N terminus of gp73ED. The term “gp28ED” means the extracellular domain of a subunit having a transmembrane domain subsequent to the C terminus of gp52SO (hereinafter, this subunit is referred to as “gp36TM”). The boundary between gp52SO and gp28ED in SEQ ID NO: 15 in the Sequence Listing exists between a serine residue at amino acid No. 376 and a phenylalanine residue at amino acid No. 377, though the boundary between gp52SO and gp28ED according to the present invention is not limited thereto. Any RX protein of the present invention can be found in a form free from a membrane such as a cell membrane and may be in a form bound to a membrane such as a cell membrane. In this context, the molecular weight means an apparent molecular weight under the non-reducing conditions of SDS-PAGE;

(ii) the RX protein causes the onset and/or exacerbation of arthritis in the joint. This exacerbation of arthritis also includes, for example, joint destruction including bone destruction; and

(iii) the RX protein comprises any one of the following amino acid, sequences (a) to (d) (hereinafter, each referred to as an “RX amino acid sequence”), consists of an amino acid sequence comprising the RX amino acid sequence, or consists of the RX amino acid sequence:

(a) the amino acid sequence represented by SEQ ID NO: 15 (FIG. 25) in the Sequence Listing:

(b) the amino acid sequence, that exhibits 86% or higher, 88% or higher, 90% or higher, 92% or higher, 94% or higher, 96% or higher, 98% or higher, or 99% or higher sequence identity to the amino acid sequence represented by SEQ ID NO: 15 (FIG. 25) in the Sequence Listing, and of a polypeptide that causes the onset and/or exacerbation of arthritis;

(c) the amino acid sequence of, that comprises an amino acid sequence represented by SEQ ID NO: 15 (FIG. 25) in the Sequence Listing having the substitution, deletion, addition, or insertion of 1 to 50, 1 to 45, 1 to 40, 1 to 35, 1 to 30, 1 to 25, 1 to 20, 1 to 15, 1 to 10, 1 to 8, 1 to 6, 1 to 5, 1 to 4, 1 to 3, 1 or 2, or 1 amino acid(s), and of a polypeptide that causes the onset and/or exacerbation of arthritis; and

(d) the amino acid sequence, that is encoded by the nucleotide sequence of a nucleic acid hybridizing under stringent conditions to a nucleic acid having a nucleotide sequence complementary to a nucleotide sequence encoding the amino acid sequence represented by SEQ ID NO: 15 (FIG. 25) in the Sequence Listing, and of a polypeptide that causes the onset and/or exacerbation of arthritis.

As mentioned above, in an aspect of the present invention, the RX protein is gp73ED, gp52SU, or a complex comprising gp52SU and gp28ED. In an aspect of the present invention, the RX protein recognized by the antibody of the present invention is gp73ED, gp52SU, or a complex comprising gp52Su and gp28ED.

Alternatively, the RX protein may be present as the whole or a portion of a homo or hetero oligo associate comprising one or two or more subunits selected from the group consisting of gp73ED and gp52SU. Such an oligo associate having the activity of causing the onset and/or exacerbation of arthritis is also included in the “RX protein” of the present invention for the sake of convenience. An antibody that recognizes this oligo complex and suppresses the onset and/or exacerbation of arthritis is also included in the “anti-RX antibody” of the present invention. In the present invention, the “complex” may also be used as the meaning of an “associate”.

SEQ ID NO: 15 (FIG. 25) in the Sequence Listing represents an exemplary amino acid sequence of gp73ED.

An exemplary amino acid sequence of gp52SU consists of ammo acid Nos. 1 (Glu) to 376 (Ser) (FIG. 27) in the amino acid sequence represented by SEQ ID NO: 15 in the Sequence Listing. An exemplary amino acid sequence of gp23ED consists of amino acid Nos. 377 (Phe) to 553 (Lys) in the ammo acid sequence represented by SEQ ID NO: 15 in the Sequence Listing. However, the amino acid sequence of the RX protein is not limited thereto, and any polypeptide having any one or the amino acid sequences (b) to (d) of polypeptides that cause the onset and/or exacerbation of arthritis in the joint is also included in the RX protein of the present invention.

The amino acid sequence and/or other properties of the RX protein may be neither the same nor homogeneous in an individual, a tissue, a body fluid, a cell, an RX protein-containing fraction, a purified or partially purified RX protein preparation, or the like, or among a plurality of individuals, tissues, cells, RX protein-containing fractions, or RX protein preparations. One individual, tissue, body fluid, cell, RX protein-containing fraction, purified or partially purified RX protein preparation, or the like may contain plural types of RX proteins differing in amino acid sequence and/or properties. Alternatively, a plurality of individuals, tissues, cells, RX protein-containing fractions, or RX protein preparations may differ in the amino acid sequence and/or other properties of the RX protein. Even such proteins differing in amino acid sequence and/or properties from each other are all encompassed by the “RX protein” of the present invention as long as the proteins possess the properties described above in (i) to (iii).

Examples of the amino acid sequence of MMTV env can include an amino acid sequence comprising 37 amino acids (amino acids corresponding to these amino acids can be found in the amino acid sequence represented by SEQ ID NO: 61) added to the C terminus of the amino acid sequence represented by SEQ ID NO: 15, and an amino acid sequence comprising the amino acid sequence represented by SEQ ID NO: 61 except for amino acid Nos. 1 to 98 (signal peptide).

(iv) The RX protein of the present invention can be obtained from a tissue, cells derived from the tissue-cultures of the cells, and the like, of a vertebrate, preferably of a mammal, more preferably of a rodent such as a mouse or a rat and a human, even more preferably of a human or a mouse. Such a tissue and cells are not particularly limited as long as they contain the RX protein. Examples thereof can include joint tissues, blood, lymph, thymus glands, spleens, and cells derived from any of those. Preferable tissues and cells are derived from animals or patients having arthritis or exhibiting similar symptoms. However, the origin of the RX protein of the present invention is not limited to those described above, and the RX protein of the present invention is also meant to include even RX proteins derived from other animal species, other tissues, other cells, or the like as long as the proteins possess the properties described above in (i) to (iii).

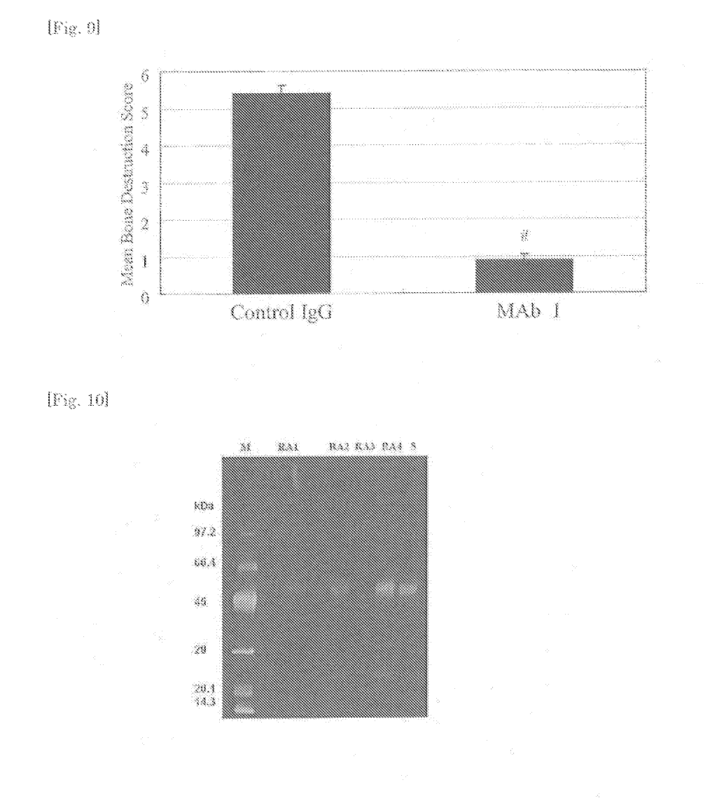

(v) The RX protein of the present invention may comprise one or two or more of the amino acid sequences described in FIG. 5 (SEQ ID NOs: 1 to 13 in the Sequence Listing). Carbamidomethyl cysteine in each of these amino acid sequences may be cysteine or cystine and may term a disulfide bond with another intramolecular or extramolecular cysteine.

The RX protein or the present invention may comprise one or two or more of the amino acid sequences described in FIG. 11 (SEQ ID NOs: 42 to 52 in the Sequence Listing). Carbamidomethyl cysteine in each of these amino acid sequences may be cysteine or cystine and may form a disulfide bond with another intramolecular or extramolecular cysteine.

The RX protein of the present invention may be a native or recombinant protein. The TX protein is also meant to include fusion products with another peptide or protein such as a carrier or a tag. The RX protein is further meant to include forms provided with chemical modification including the addition of a polymer such as PEG and/or with biological modification including sugar chain modification. Moreover, the RX protein of the present invention is meant to include an RX protein fragment. An RX protein fragment possessing the properties described above in (ii) is referred to as a functional fragment of the RX protein.

The RX protein of the present invention ca be detected in an individual affected with arthritis or autoimmune disease such as rheumatoid arthritis and/or in a collagen-induced arthritis mouse model. for example, a collagen-induced arthritis model may be prepared using a DBA/1 mouse strain. In such a case, the RX protein of the present invention can be detected in the blood of the model. However, the detection of the RX protein differs among species, strains, and individuals.

### (2-2) Gene

The RX gene of the present invention comprises any one of the following nucleotide sequences (a) to (c) (hereinafter, each referred to as an “RX gene sequence”), consists of a nucleotide sequence comprising the RX gene sequence, or consists of the RX gene sequence:

(a) the nucleotide sequence represented by SEQ ID NO: 14 (FIG. 24) in the Sequence Listing;

(b) a nucleotide sequence that hybridizes under stringent conditions to a nucleic acid consisting of a nucleotide sequence complementary to the nucleotide sequence represented by SEQ ID NO: 14 (FIG. 24) in the Sequence Listing and encodes the amino acid sequence of a polypeptide causing the onset and/or exacerbation of arthritis; and

(c) a nucleotide sequence that comprises a nucleotide sequence represented by SEQ ID NO: 14 (FIG. 24) in the Sequence Listing having the substitution, deletion, addition, or insertion of 1 to 150, 1 to 140, 1 to 130, 1 to 120, 1 to 110, 1 to 100, 1 to 90, 1 to 80, 1 to 70, 1 to 60, 1 to 50, 1 to 45, 1 to 40, 1 to 30, 1 to 25, 1 to 20, 1 to 15, 1 to 10, 1 to 8, 1 to 6, 1 to 5, 1 to 4, 1 to 3, 1 or 2, or 1 bases(s) and encodes the amino acid sequence of a polypeptide causing the onset and/or exacerbation of arthritis.

In one aspect of the present invention, the RX protein consists of a single chain polypeptide or gp73 ED, as mentioned above. In another aspect, gp52SU and gp28ED associate with each other to form a complex. In one aspect of the present invention, the RX gene encodes gp73ED or encodes both gp52SU and gp28ED. In an alternative aspect, the RX gene encodes only gp52SU.

The RX gene is overexpressed in autoimmune disease, for example, rheumatoid arthritis. In particular, the RX gene is overexpressed in a joint tissue or blood fraction derived from a rheumatoid arthritis patient or a rheumatoid arthritis animal model, for example, a joint synovium or plasma fraction derived from the patient.

The expression and expression level of the RX gene may be assayed with either an RX gene transcript or the RX protein as an index. The former index can be determined by RT-PCR, Northern blot hybridization, or the like, while the latter index can be determined by immunoassay (e.g., enzyme-linked immunosorbent assay; hereinafter, referred to as “ELISA”) or the like.

SEQ ID NO: 14 (FIG. 24) in the Sequence Listing represents an exemplary nucleotide sequence encoding the amino acid sequence of gp73ED. An exemplary nucleotide sequence encoding the amino acid sequence of gp52SU consists of the nucleotides Nos. 1 to 1128 (FIG. 26) in the nucleotide sequence represented by SEQ ID NO: 14 in the Sequence Listing. An exemplary nucleotide sequence encoding the amino acid sequence of gp28ED consists of the nucleotides Nos. 1129 to 1659 in the nucleotide sequence represented by SEQ ID NO: 14 in the Sequence Listing. However, the nucleotide sequence of the RX gene is not limited thereto, and any gene having either of the nucleotide sequences (b) or (c) encoding the amino acid sequences of polypeptides that cause the onset and/or exacerbation of arthritis in the joint is also included in the RX gene of the present invention.

### (2-3) Preparation of Protein

The RX protein of the present invention can be prepared by purification or isolation from animal tissues (including body fluids), cells derived from the tissues, or cultures of the cells, gene recombination, in vitro translation, chemical synthesis, etc.

### (2-3-1) Purification or Isolation of Native RX

The native RX protein can be purified or isolated from, for example, tissues (including body fluids, cells, etc.) derived from patients or non-human animals affected with autoimmune disease such as RA or arthritis, cells derived from the tissues, or cultures of the cells as long as they contain the RX protein of the present invention. Such non-human animals also include animal models of these diseases. The animals subjected to model preparation can be any vertebrate without particular limitations and are preferably mammals, more preferably rodents such as mice or rats, even more preferably mice or rats. The tissues and cells of such patients or animal models are not particularly limited as long as they contain the RX protein. Examples thereof can include joint tissues, blood, lymph, thymus glands, spleens, and cells derived from any of those. Preferable tissues end cells are derived from patients or animal models having arthritis or exhibiting similar symptoms. However, the origin of the RX protein of the present invention is not limited to those described above, and the RX protein of the present invention may be derived from other animal species, other tissues, other cells, or the like.

The purification or isolation from such tissues, cells, cell cultures, or the like can be performed by the combination of approaches well known by those skilled in the art, such as fractionation and chromatography. Such approaches include, but are not limited to, salting out, gel filtration, ion-exchange chromatography, affinity chromatography, hydrophobic chromatography, normal-phase or reverse-phase chromatography, and the like. A column for affinity chromatography can be prepared by packing with an affinity gel cross-linked with an anti-RX monoclonal antibody. A crude or partially purified fraction containing the RX protein is added to this column. Subsequently, non-specific adsorbed matter is removed with sterilized phosphate-buffered saline (PBS), and a buffer solution for elution can then be added thereto to thereby selectively collect the RX protein. The solution containing the RX protein can be subjected to gel filtration or to buffer replacement and/or concentration using a concentrator such as Centriprep.

The RX protein can be prepared from cells derived from the arthritis mouse model, for example, by the following method:

In adjuvant, pristane, an anti-collagen antibody, type II collagen, or the like can be administered to non-human animals such as mice to thereby artificially cause the onset of arthritis.

The malleolar joint tissue of a hindlimb is aseptically collected from an animal with serious arthritis. Cells can be extracted from the tissue slices in a culture dish. The obtained cells can be cultured for 1 to 6 months in a culture medium to obtain native RX-producing cells. An animal can be immunized with the cells themselves or concentrates of the culture supernatant of the cells to produce a monoclonal antibody. Examples of the animal species used in the immunization can include rodents such as rats and mice. After final immunization, antibody-producing cells are collected from the spleen of the animal and fused with myeloma cells to obtain fusion cells (hybridomas). The antibody-producing cells are not limited to the spleen cells, and the cells of thymus glands or lymph nodes may also be used. A myeloma cell line known in the art such as 8-653, P3, or NS-1 may be used in the cell fusion. For example, polyethylene glycol (PEG) or Hemagglutinating virus of Japan (HVJ) can be used as a cell fusion promoter. If necessary, an aid such as dimethyl sulfoxide may be added. The hybridomas can be screened for a single clone producing the antibody specifically binding to the RX protein by limiting dilution analysis. In addition to the limiting dilution analysis, a cytofluorometer or a cell separation apparatus such as ClonePix (Molecular Devices, Inc. (Genetrix) may be used in the cloning or antibody-producing cells. The hybridoma thus prepared can be cultured in a usual medium and can also be stored for a long period in liquid nitrogen using a cell storage solution (e.g., Cell Banker; Juji Field Inc.) or the like. The desired antibody can be obtained from the hybridoma by the application of, for example, a method involving preparing a culture supernatant using a flask for antibody production (e.g., CL-1000 flask; Section, Dickinson and Company) or a method involving inoculating the hybridoma in a mammal compatible with the hybridoma to obtain ascites. The anti-RX monoclonal antibody can be purified from the solution containing the antibody obtained by any of these methods. A column packed with an affinity gel cross-linked with the obtained anti-RX monoclonal antibody can be used to selectively concentrate the RX protein contained in the RX protein-containing fraction. Alternatively, the RX protein may be purified or isolated by an appropriate combination with other approaches for fractionation or purification.

### (2-3-2) Preparation of Recombinant RX Protein

The RX protein of the present invention can also be prepared in a recombinant form. Specifically, host cells are transfected with a gene encoding the amino acid sequence of the RX protein or an RX protein fragment, and the RX protein can be collected from cultures of the cells. For example, the RX gene or its fragment is inserted into an expression vector. Subsequently, prokaryotic or eukaryotic host cells are transfected with the resulting recombinant vector, and the obtained recombinant cells can be incubated to thereby express the RX protein. An expression pattern known in the art, such as secretion expression, intracellular expression of soluble forms, or expression in inclusion body forms can be used. Also, the RX protein can be expressed not only as a molecule having the same amino terminus (N terminus) and/or carboxy terminus (C terminus) as native ones, but also as a fusion protein with a secretory signal, an intracellular localization signal, a tag for affinity purification, or a partner peptide. The RX protein can be purified or isolated from such recombinant cell cultures by an appropriate combination of operations such as fractionation and chromatography described in (2-3-1) Purification or isolation of native RX protein.

The RX gene or its fragment can be prepared by a method well known by those skilled in the art,

Examples thereof can include: polymerase chain reaction (hereinafter, referred to as “PCR”; Saiki, R. K., at al., Science (1988) 239, p. 483-489) with a cDNA library for RX gene expression as a template using one set of primers capable of specifically amplifying the sequence; reverse transcription PCR (hereinafter, referred to as “RT-PCR”) with an mRNA fraction for RX gene expression as a template using a primer capable of reverse-transcribing the sequence and one set or primers capable of specifically amplifying the sequence; expression cloning using immunoassay; and cDNA cloning using the partial amino acid sequence of purified RX protein.

### (2-3-3) In-vitro Translation

The RX protein of the present invention can also be prepared by in vitro translation. Such a translation method is not particularly limited as long as the method employs a cell-free translation system involving enzymes necessary for transcription and translation, substrates, and energy substances. Examples thereof can include a method using Rapid Translation System (RTS) manufactured by Roche Diagnostics K. K.

### (2-3-4) Chemical Synthesis

The RX protein of the present invention can also be prepared by chemical synthesis. Examples of the chemical synthesis method can include solid-phase peptide synthesis methods such as Fmoc and Boc synthesis methods.

### 3. Antibody

### (3-1) Type of Antibody

The antibodies of the present invention may be either monoclonal or polyclonal antibodies. Examples of the monoclonal antibody of the present invention can include a non-human animal-derived antibody (non-human animal antibody), a human-derived antibody (human antibody), a chimeric antibody, and a humanized antibody.

Examples of the non-human animal antibody can include antibodies derived from vertebrates such as mammals and birds. Examples of the mammal-derived antibody can include rodent-derived antibodies such as mouse antibodies and rat antibodies. Examples of the bird-derived antibody can include chicken antibodies.

Examples of the chimeric antibody can include, but are not limited to, an antibody comprising non-human animal antibody-derived variable regions bound with human antibody (human immunoglobulin) constant regions. Examples of the non-human animal antibody-derived variable regions can include heavy and light chain variable regions derived from MAb1, MAb2, MAb3, and/or MAb4 described later.

Examples of the humanized antibody can include, but are not limited to, a human antibody (human immunoglobulin variable regions) grafted with CDRs in the variable regions of a non-human animal antibody, a human antibody grafted with the CDRs as well as with partial sequences of framework regions of a non-human animal antibody, and an antibody having human antibody amino acid(s) substituted for one or two or more non-human animal antibody-derived amino acid(s) in any of these humanized antibodies. Examples of the CDRs in the variable regions of a non-human animal antibody can include CDRH1 to CDRH3 in the heavy chain variable region and CDRL1 to CDRL3 in the light chain variable region derived from MAb1, MAb2, MAb3, and/or MAb4 described later.

The human antibody is not particularly limited as long as the antibody recognizes the antigen of the present invention. Examples thereof can include a human antibody having the CDRs of the antibody of the present invention. Examples of the CDRs of the human antibody of the present invention can include CDRH1 to CDRH3 in the heavy chain variable regions and CDRL1 to CDRL3 in the light chain variable region derived from MAb1, MAb2, MAb3, and/or MAb4 described later.

The antibody according to the present invention may be comprised of portions derived from a plurality of different antibodies. Examples of such an antibody can include an antibody comprising heavy and/or light chains exchanged among a plurality of different antibodies, an antibody comprising full-length heavy and/or light chains exchanged thereamong, an antibody comprising variable or constant regions exchanged thereamong, and an antibody comprising all or some CDRs exchanged thereamong. The heavy and light chain variable regions of the chimeric antibody may be derived from different antibodies of the present invention. CDRH1 to CDRH3 and CDRL1 to CDRL3 in the heavy and light chain variable regions of the humanized antibody may be derived from two or more different antibodies of the present invention. CDRH1 to CDRH3 and CDRL1to CDRL3 in the heavy and light chain variable regions of the human antibody may be a combination of CDRs carried by two or more different antibodies of the present invention.

Examples of the isotype of the monoclonal antibody of the present invention can include, but are not particularly limited to, IgG such as IgG1, IgG2, IgG3, and IgG4, IgM, IgA such as IgA1 and IgA2, IgD, and IgE and can preferably include IgG and IgM. The isotype and subclass of the monoclonal antibody can be determined by, for example, an Ouchterlony test, ELISA, or radio immunoassay (hereinafter, referred to as “RIA”). A commercially available kit for identification (e.g., Mouse Typer Kit; Bio-Rad Laboratories, Inc.) may be used.

### (3-2) Antigen Binding Activity of Antibody

The antibody of the present invention recognizes the RX protein. In other words, the antibody of the present invention binds to the RX protein. Such an antibody is referred to as an “anti-RX antibody”. Preferably, the antibody of the present invention specifically recognizes the RX protein. In other words, preferably, the antibody of the present invention specifically binds to the RX protein.

In the present invention, the “specific recognition”, i.e., “specific binding”, means binding which is not non-specific adsorption. Examples of criteria for determination of whether binding is specific or not can include a dissociation constant (hereinafter, referred to as “KD”. The preferable antibody of the present invention has a KD value of 1×10⁻⁵ or lower, 5×10⁻⁶ or lower, 2×10⁻⁶ or lower, 2×10⁻⁷ or lower, or 1×10⁻⁷ or lower, even more preferably 5×10⁻⁸ or lower, 2×10⁻⁹ or lower, or 1×10⁻⁸ or lower, further more preferably 5×10⁻⁹ or lower, 2×10⁻⁹ or lower, or 1×10⁻⁹ or lower, most preferably 5×10⁻¹⁰ or lower, 2×10⁻¹⁰ or lower, or 1×10⁻¹⁰ or lower for the RX protein.

In the present invention, the binding of the antibody to the antigen can be assayed or determined by ELISA, RIA, surface plasmon resonance (hereinafter, referred to as “SPR”) analysis, or the like. Examples of equipment used in the SPR analysis can include BIAcore™ (GE Healthcare Bio-Sciences Corp.), ProteOn™ (Bio-Rad Laboratories, Inc.), SPR-Navi™ (BioNavis Oy Ltd.), Spreeta™ (Texas Instruments Inc.), SPRi-Plex IT™ (Horiba, Ltd.), and Autolab SPR™ (Metrohm Japan Ltd.) The binding of the antibody to the antigen expressed on cell surface can be assayed by flow cytometry or the like.

The RX protein so which the antibody of the present invention binds causes the onset and/or exacerbation of arthritis in the joint, as mentioned above. This exacerbation of arthritis also includes, for example, joint destruction including bone destruction. The onset and/or exacerbation of arthritis are found in, for example, the joint of a collagen-induced arthritis non-human animal model, preferably a collagen-induced arthritis mouse model.

(3-3) Biological Activity of Antibody

In a preferable aspect, the antibody of the present invention has an arthritis suppressive activity (anti-arthritic activity).

The anti-arthritic activity means the activity of suppressing the onset and/or exacerbation of arthritis.

The anti-arthritic activity can be evaluated on the basis of the degree of improvement in arthritis score according to a routine method. For example, collagen-induced arthritis mice are widely used as disease models of RA and arthritis. The degree of arthritis in each limb can be scored for assessment as follows: 0=no sign of arthritis, 1=erythema and/or edema developed in one joint, 2=erythema and/or edema developed in two joints, 3=erythema and/or edema developed in the whole limb, and 4=joint deformity or rigidity. The overall points from the assessment of all limbs can be evaluated as the arthritis score of the individual.

In the present invention, the phrase “having an anti-arthritic function” refers to reduction in the arthritis score (described in the preceding paragraph) of the individual compared with a control group. N% or more reduction in the arthritis score of the individual compared with a control group is referred to as “having N% or more anti-arthritic function”. For example, 30% or more reduction in the score is referred to as “having 30% or more anti-arthritic function”, while 50% or more reduction therein is referred to as “having 50% or more anti-arthritic function”. This arthritis score is preferably a score determined in a collagen-induced arthritis non-human animal model, more preferably a score determined in a collagen-induced arthritis mouse model, even more preferably an arthritis score determined by the method described in paragraphs a) and b) of Example 6. The number of days from the sensitization of the mouse model to the determination is 30 days or longer, preferably 40 days or longer. When the test compound is an antibody, the control group is preferably a group (control IgG antibody-administered group) that has received IgG (hereinafter, referred to as a “control IgG antibody”) purified from normal rat serum as a negative control.

The antibody of the present invention not only has the anti-arthritic activity but may further have the activity of suppressing bone destruction. The bone destruction associated with RA generally includes, for example, development of marginal bone erosions or joint space narrowing by pannus, formation of cysts of subchondral bones, bone atrophy accompanied by periarticular osteoporotic alteration, osteonecrosis, and fragility or pathological fracture caused by combinations thereof. The degree of improvement in bone destruction can be determined as follows: in the case of, for example, an animal model, its limbs are subjected to soft X-ray photography after the completion of experiments. Each site such as calcaneum, tarsal bone, or metatarsus can be scored according to, for example, 0=normal, 1=mild, 2=moderate, and 3=severe. Also in the case of a patient, his or her limbs may be subjected to X-ray photography before or after medication or during treatment and scored in the same way as above. The suppression of this bone destruction can be evaluated using, for example, a collagen-induced arthritis non-human animal model, preferably a collagen-induced arthritis mouse model.

Alternatively, the anti-arthritic activity may be assayed or determined with an arthritis biomarker (arthritis marker), a bone destruction marker, or the like as an index.

The arthritis marker is not particularly limited as long as the index correlates with the severity of arthritis, the degree of its progression, the degree of curing brought about by treatment, etc. Examples thereof can include: inflammatory cytokines such as IL-1β, IL-6, and IL-12; chemokines such as MCP-1, macrophage inflammatory protein 1 alpha (MIP-1α), and regulated on activation, normal T cell expressed and secreted (RANTES); lipid mediators; enzymes such as matrix metalloproteinase-3 (MMP-3); and antibodies such as anti-cyclic citrullinated peptide (CCP) antibodies.

The antibody of the present invention may have a cytokine production inhibitory activity and may more preferably have an inhibitory activity against the production of an inflammatory cytokine and/or a chemokine. This cytokine production inhibitory activity is preferably found in an inflamed (body) region and can be evaluated, for example, in one inflamed region of a collagen-induced arthritis non-human animal model, preferably a collagen-induced, arthritis mouse model.

The anti-RX antibody of the present invention may have an inhibitory activity against the production (including the activity of inhibiting the promotion of production) of a bone destruction marker in blood. Examples of such a bone destruction marker can include type II collagen telopeptide, type I collagen telopeptide, and MPP9. These markers can each be assayed using a commercially available ELISA kit, though the assay method is not limited thereto. The joint destruction suppressive activity of the anti-RX antibody of the present invention may be assayed directly by image analysis using an imaging technique such as microcomputer tomography (micro-CT). In the case of an animal model, its limbs can be subjected to micro-CT photography after the completion of experiments or over time and evaluated by scoring according to the severity of joint destruction. The assay approach is not limited thereto, and any imaging technique capable of evaluating joint destruction can be used.

The antibody of the present invention may have the activity of decreasing the amount of the RX protein and may preferably have the activity of decreasing the amount of the RX protein in autoimmune disease or arthritis. This decrease in the amount of the RX protein can be evaluated using, for example, a collagen-induced arthritis non-human animal model, preferably a collagen-induced arthritis mouse model.

The anti-RX antibody of the present invention may have an antibody-dependent cellular cytotoxic (ADCC) activity and/or a complement-dependent cytotoxic (CDC) activity and/or an antibody-dependent cell-mediated phagocytosis (ADCP) activity. The ADCC, CDC, and ADCP activities can be assayed by any method known in the art.

Cells (target cells) each expressing the antigen of interest and effector cells that kill the target cells are used in the ADCC activity assay. Each effector cell recognizes the Fc region of an antibody bound with the target cell via an Fcγ receptor. The effector cell kills the target cell by signals transmitted from the Fcγ receptor. Human NK cells are used as the effector cells in the assay of the ADCC activity of an antibody having a human-derived Fc region. The human NK cells can be prepared from human peripheral mononuclear blood cells (PMBCs) by any method known in the art. Alternatively, PMBCs themselves may be used as the effector cells.

Cells (target cells) each expressing the antigen of interest and effector cells (e.g., monocytes or macrophages) that phagocytize the target cells are used in the ADCP activity assay. These effector cells can be prepared by separating a monocyte fraction from human peripheral mononuclear blood cells (PMBCs) by any method known in the art and inducing its differentiation into macrophages by any method known in the art.

### (3-4) Cell Used in Antibody Assay

The anti-RX antibody of the present invention may be subjected to an evaluation system using a cell line or primary cultured cells that exhibit some induced response to the RX protein. Examples of such a cell line can include a human synovium-derived cell line (SW982 cells; ATCC Ho. KTB-93) and a mouse macrophage-like cell line (RAW 264.7; ATCC NO. TTB-71). Examples of such primary cultured cells can include mouse bone marrow cells. Examples of an index for cell activation induced by the RX protein can include calcium influx (Ca influx) and cytokine production from the cell. The effect or the anti-RX antibody on such cell activation may be evaluated by the evaluation system mentioned above. Intracellular calcium concentration may be measured using, for example, FLUO-4 kit NW Calcium assay kit (Cat. #F36206, Invitrogen Corp.), The cytokines, etc., can be assayed using a commercially available kit. However, the assay system is not limited thereto, and any system capable of assaying cell response induced by the RX protein and suppression thereof can be used).

### (3-5) Monoclonal Antibody

Antibody 1 (MAb1) is a monoclonal antibody obtained by the intraperitoneal and intradermal administration of a mixtures of ADSF cells and a concentrated solution of the culture supernatant thereof to a WKY/NCrj rat and one of its soles, respectively, according to the method described in paragraph a) or Example 2.

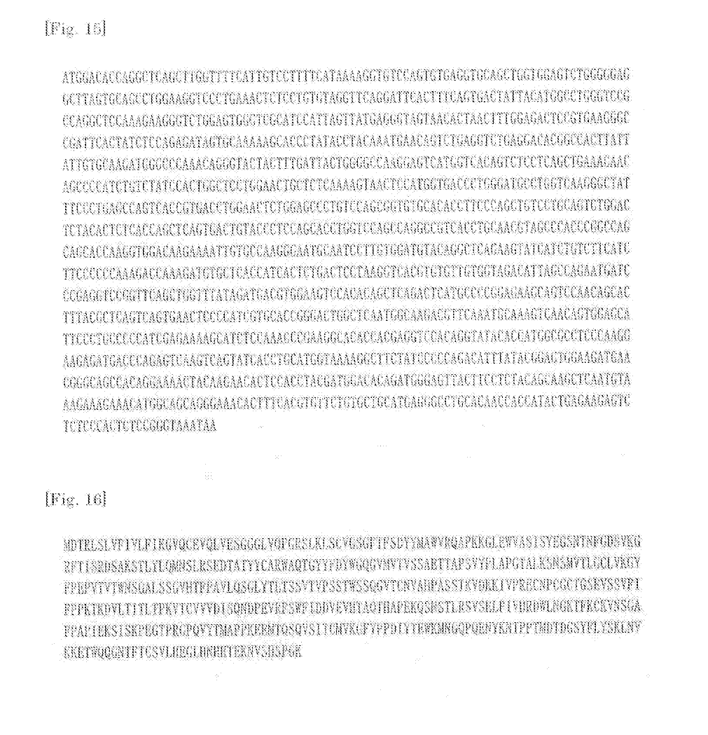

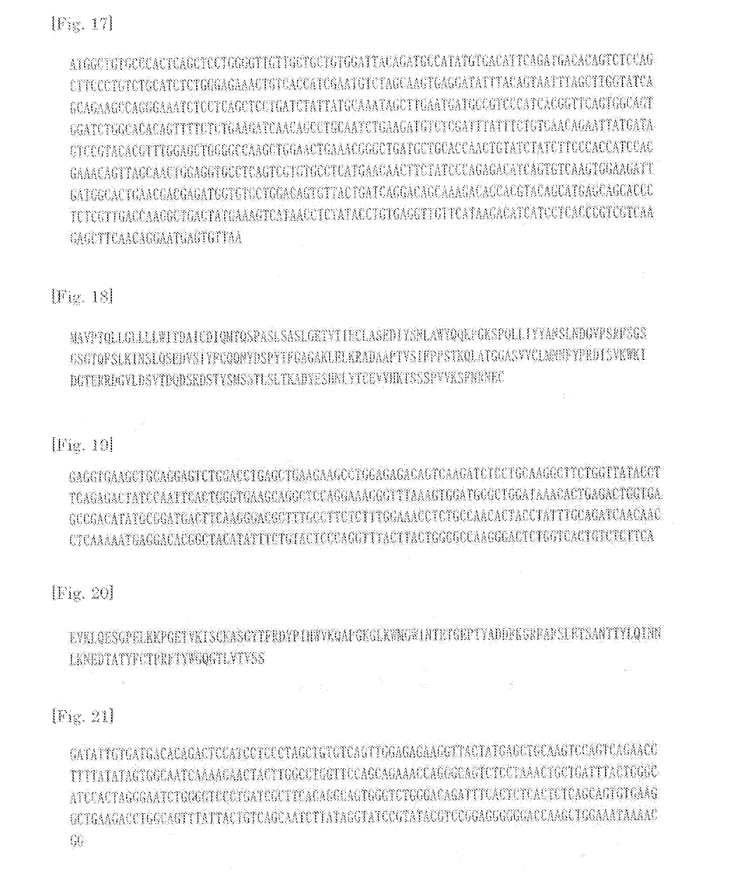

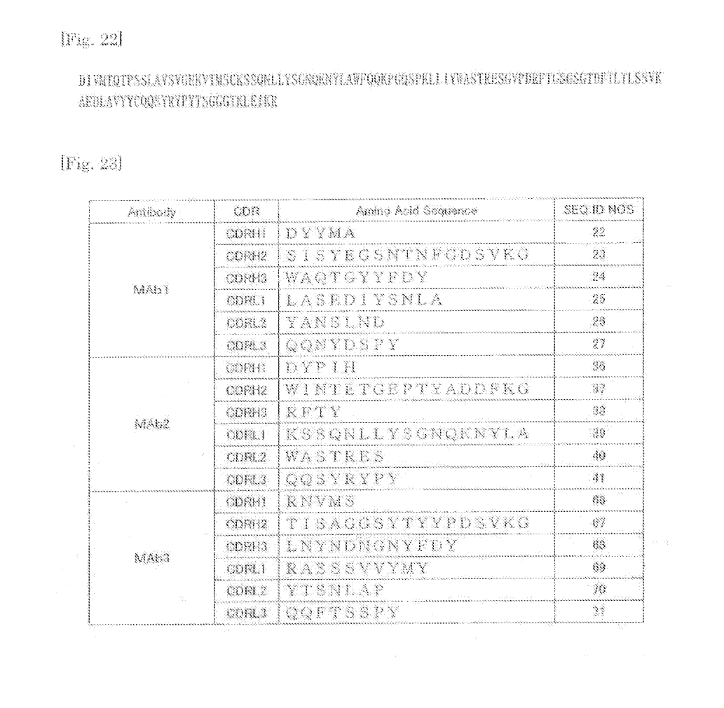

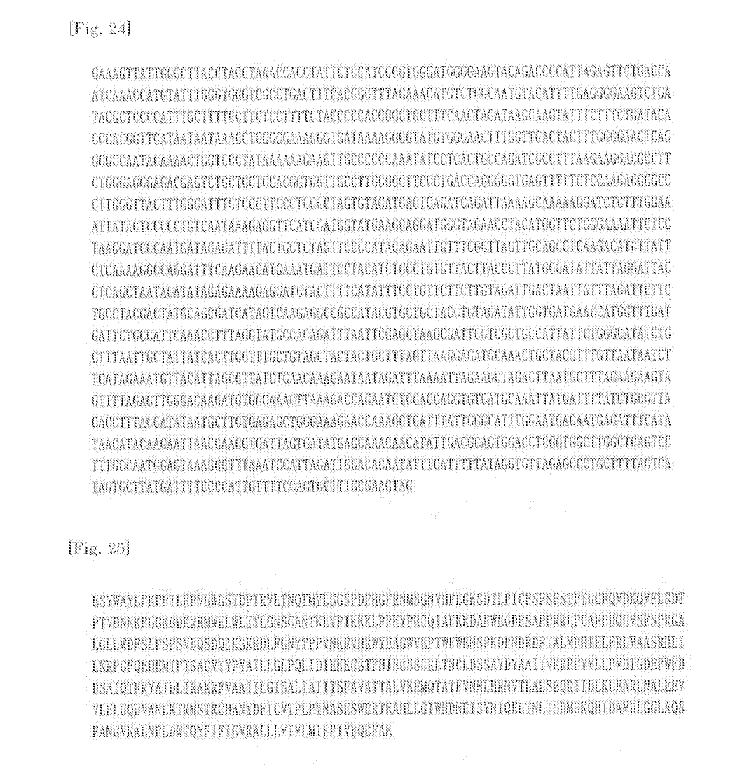

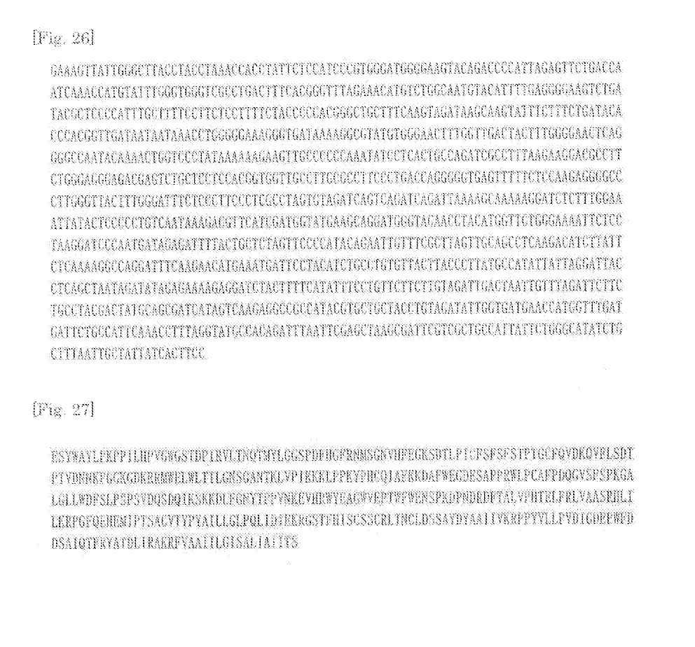

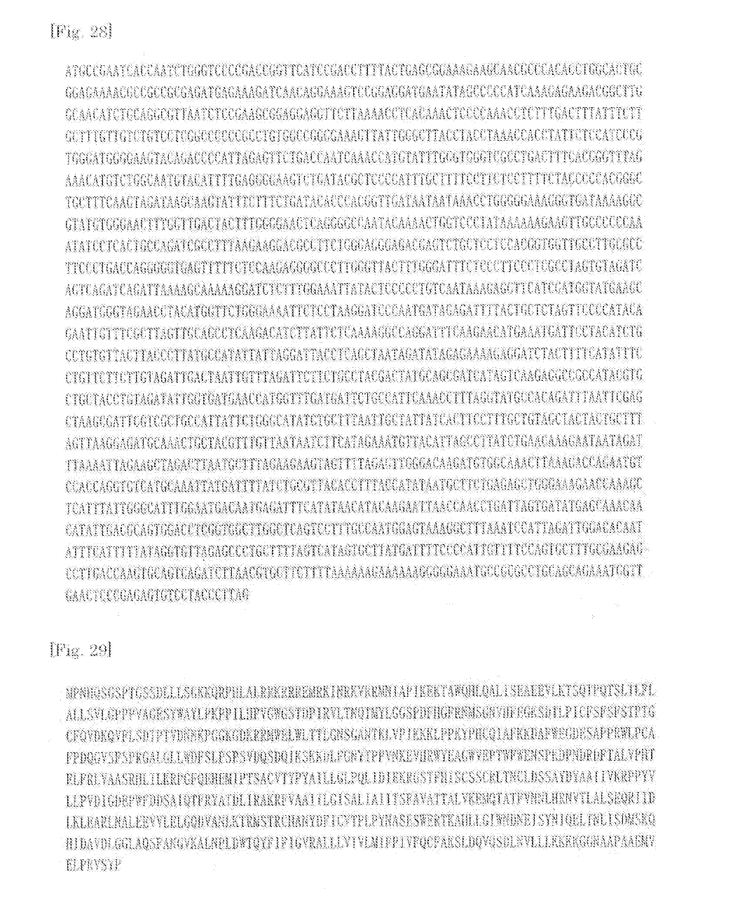

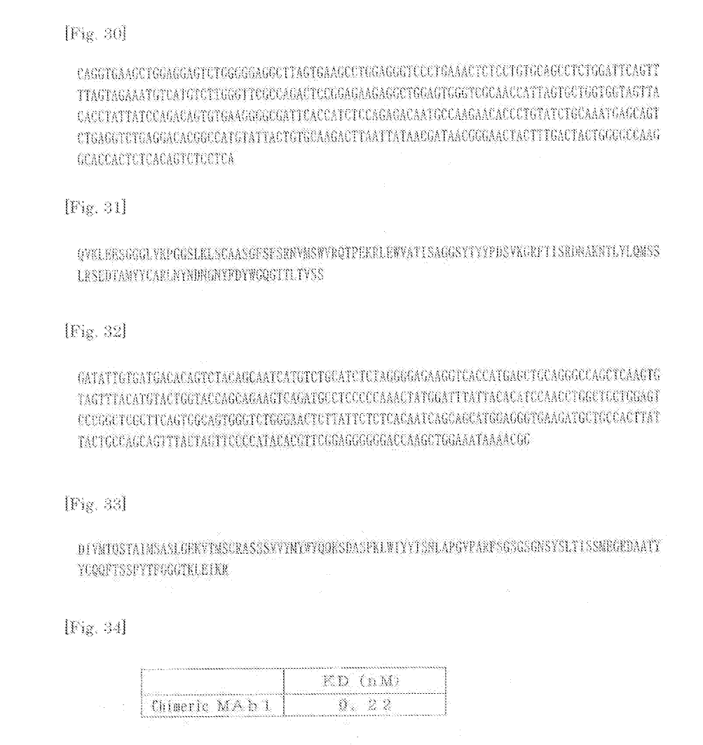

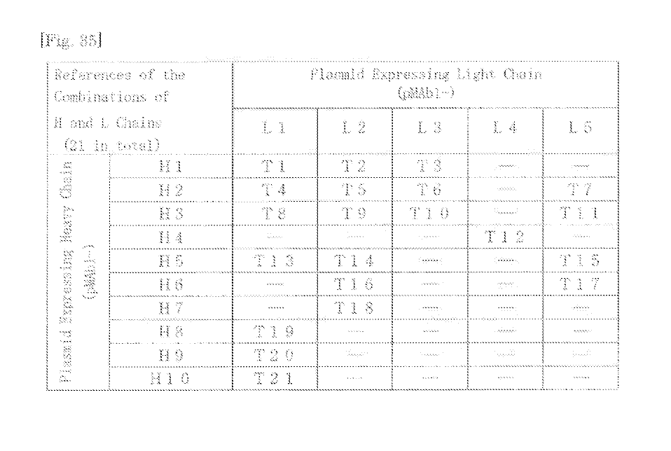

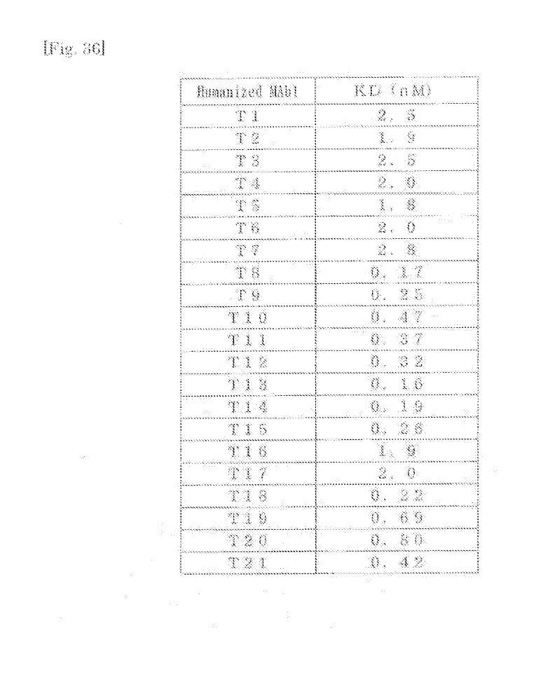

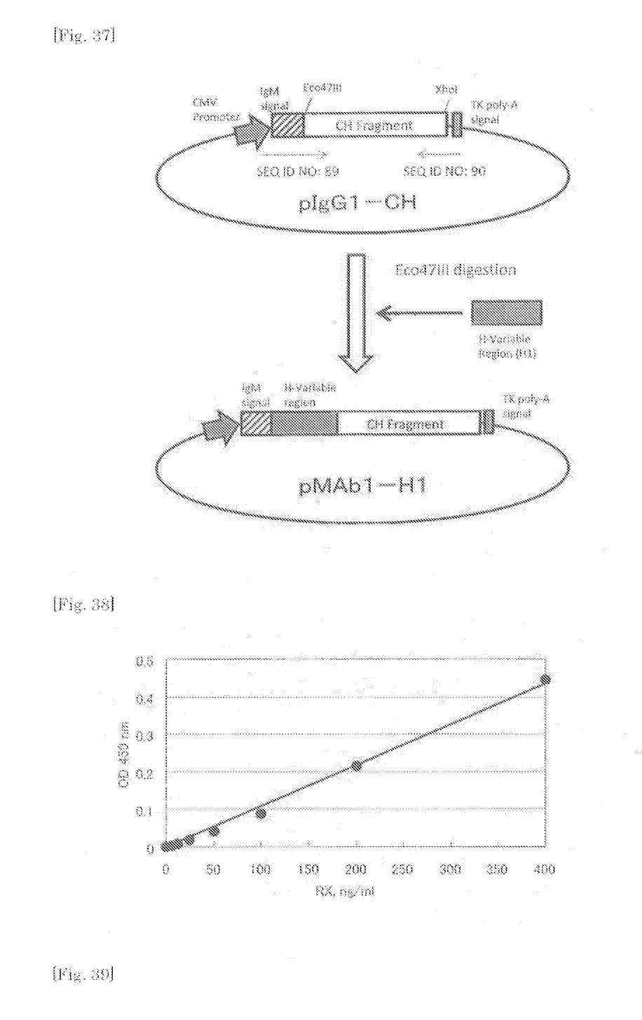

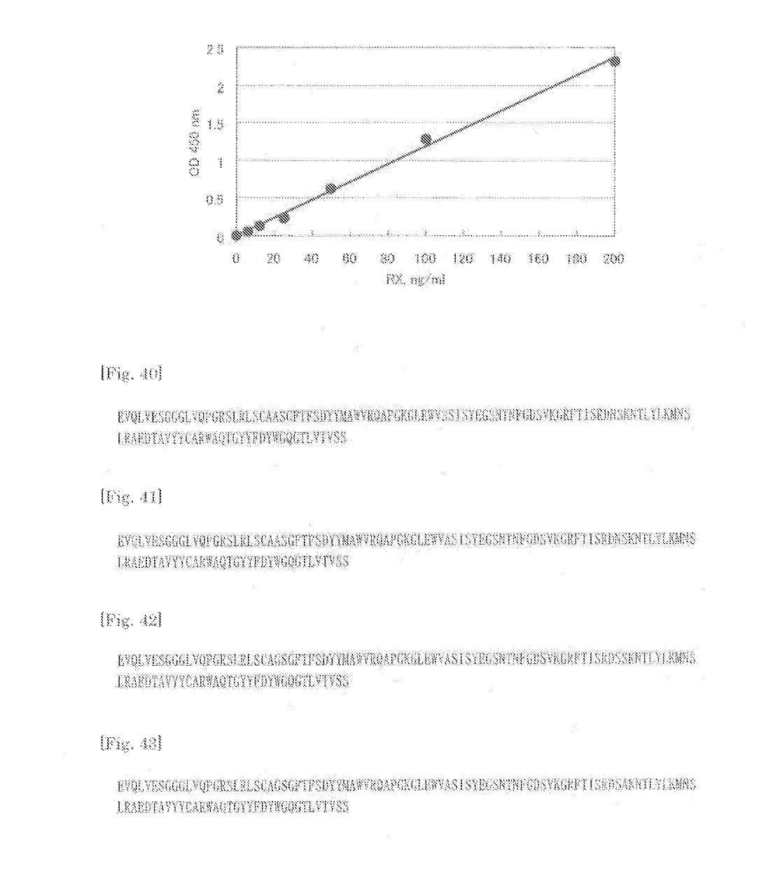

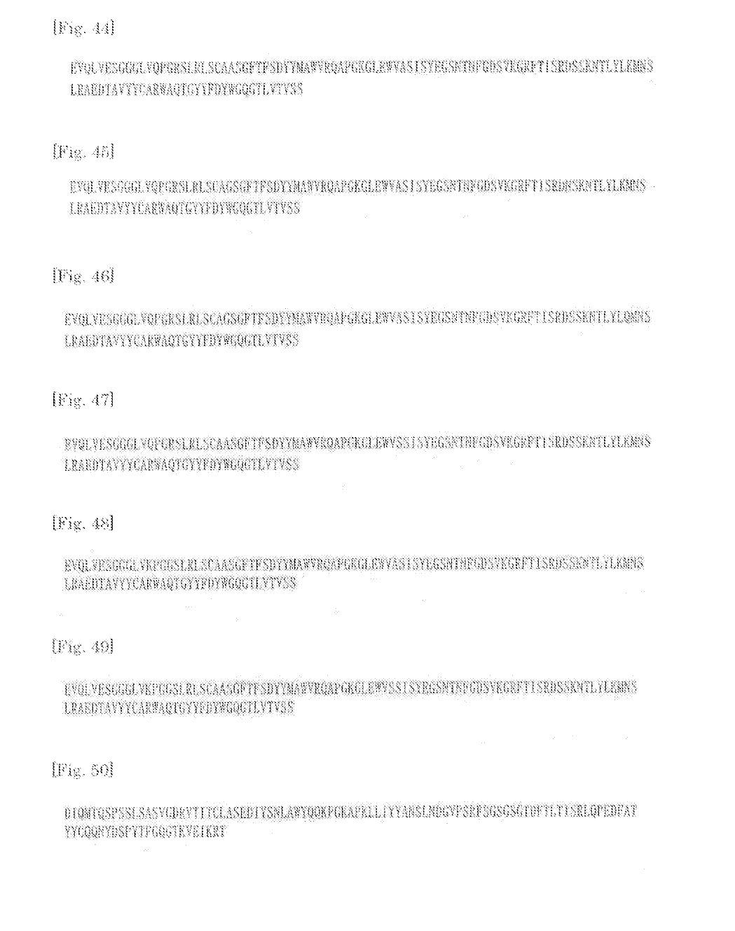

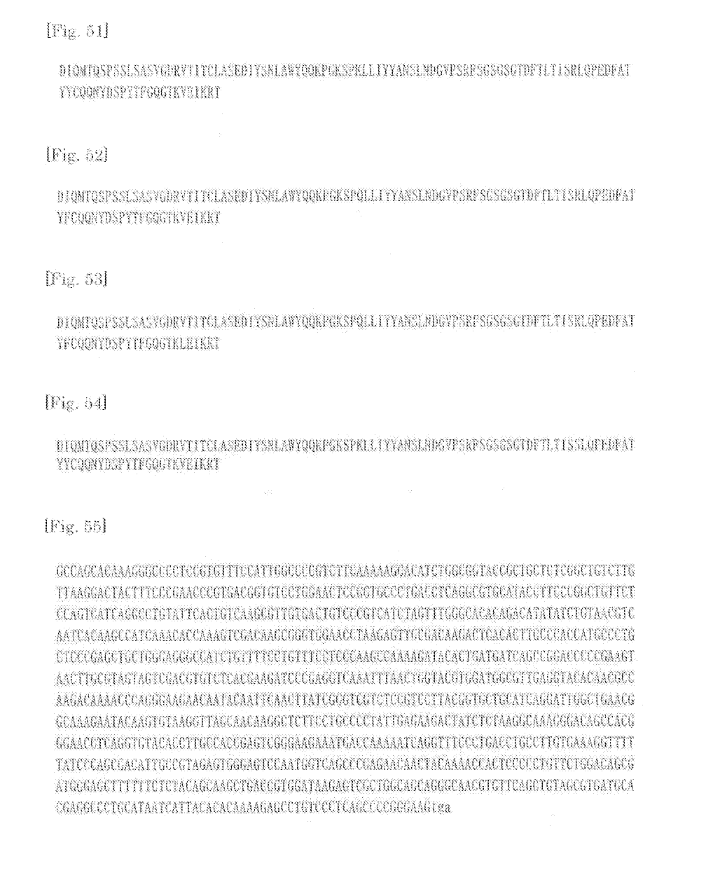

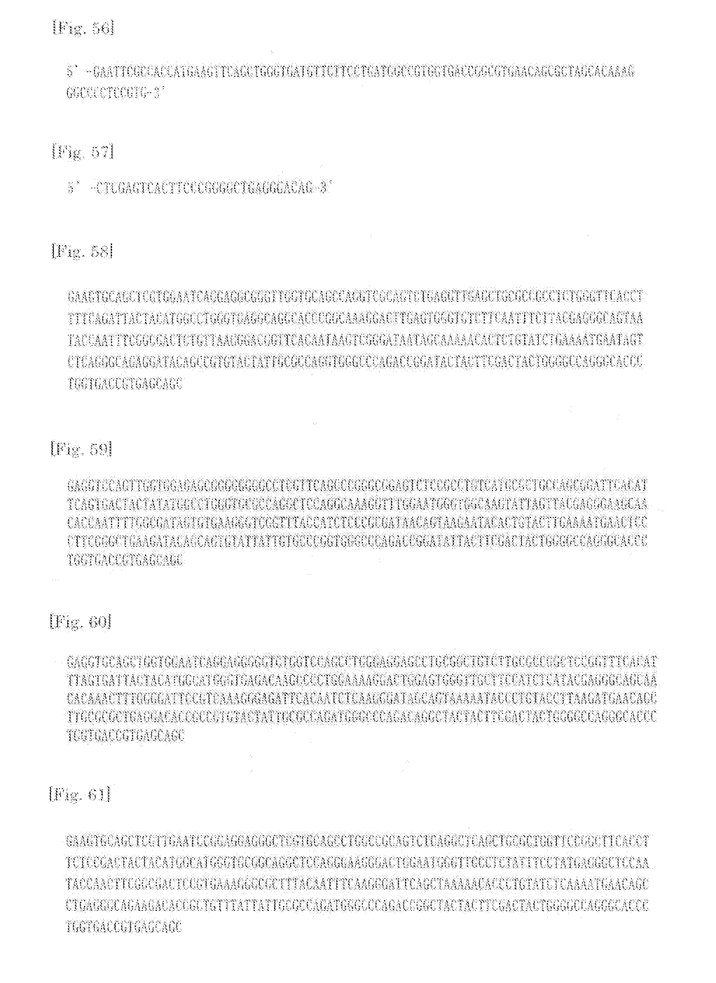

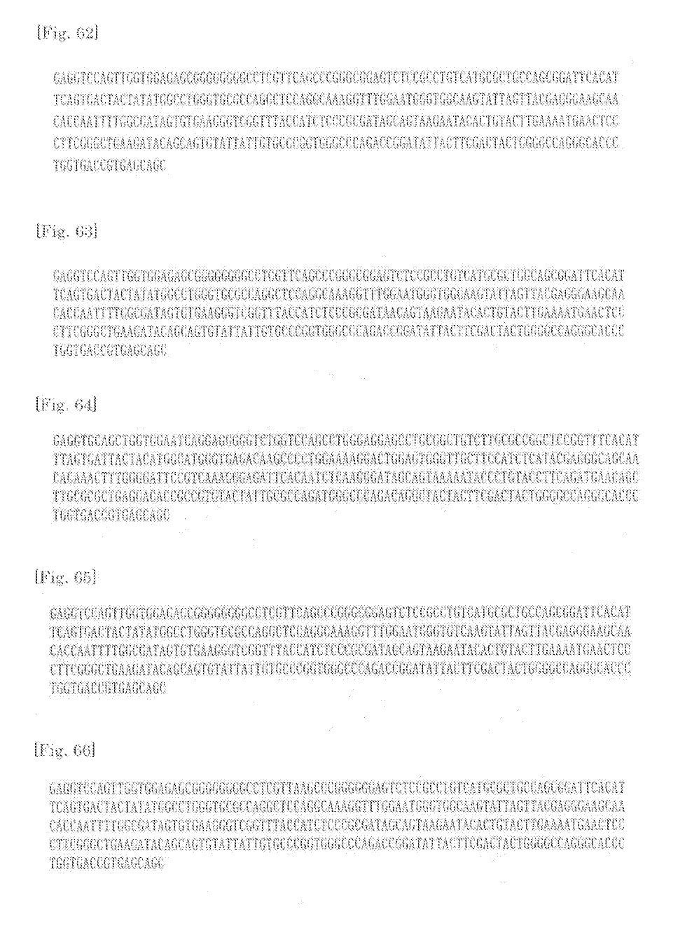

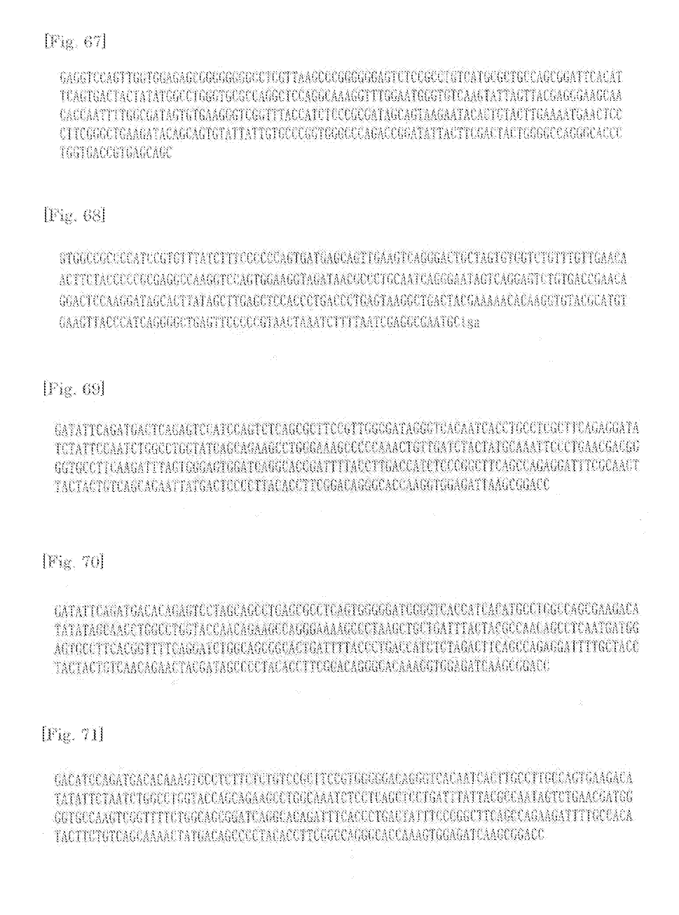

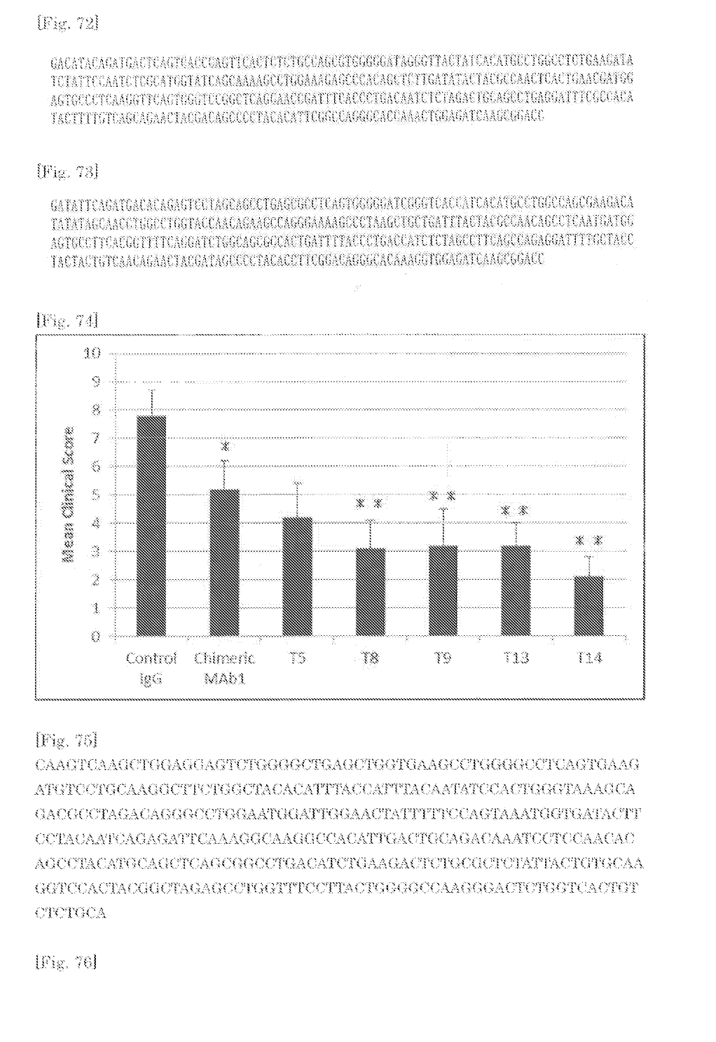

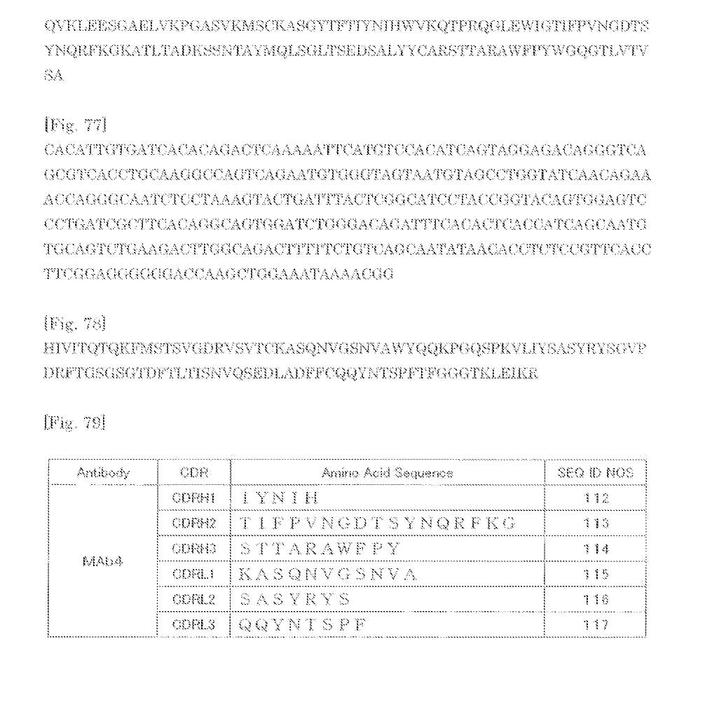

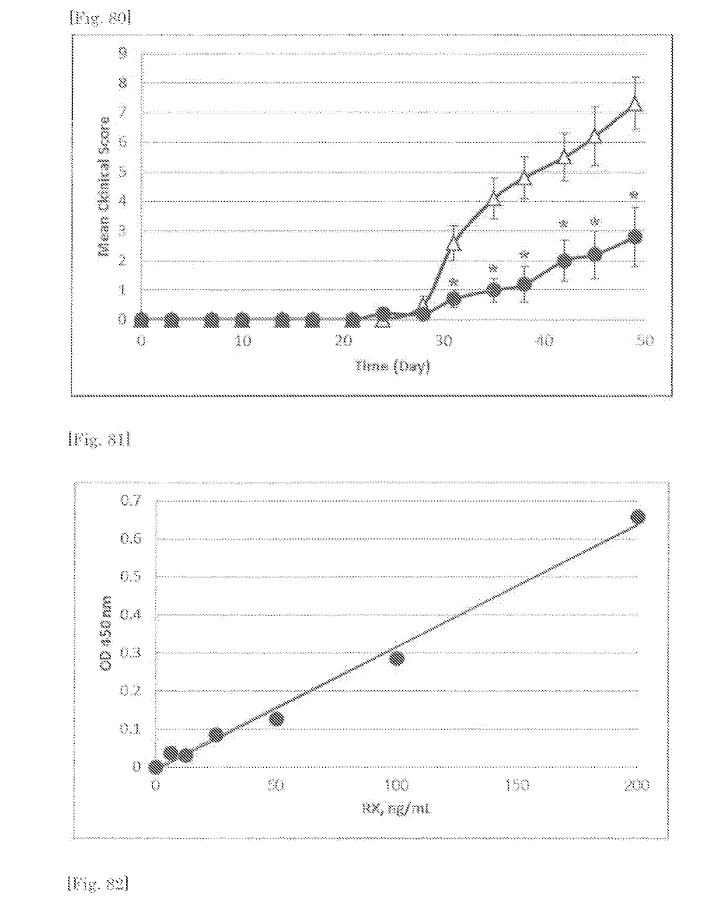

The nucleotide sequence and amino acid sequence of the heavy chain of MAb1 are described in SEQ ID NOs: 18 (FIG. 15) and 19 (FIG. 16), respectively, in the Sequence Listing. The nucleotide sequence and amino acid sequence of the light chain of MAb1 are described in SEQ ID NOs: 20 (FIG. 17) and 21 (FIG. 18), respectively, in the Sequence Listing. The nucleotide sequence of the heavy chain variable region of MAb1 corresponds to the nucleotides Nos. 58 to 414 in SEQ ID NO: 18 (FIG. 15) in the Sequence Listing. The amino acid sequence thereof corresponds to amino acid Nos. 20 to 138 in SEQ ID NO: 19 (FIG. 16) in the Sequence Listing. The nucleotide sequence of the heavy chain constant region of MAb1 corresponds to the nucleotides Nos. 415 to 1383 in SEQ ID NO: 18 (FIG. 15) in the Sequence Listing. The amino acid sequence thereof corresponds to amino acid Nos. 139 to 460 in SEQ ID NO: 19 (FIG. 16) in the Sequence Listing. The nucleotide sequence of the light chain variable region of MAb1 corresponds to the nucleotides Nos. 61 to 387 in SEQ ID NO: 20 (FIG. 17) in the Sequence Listing. The amino acid sequence thereof corresponds to amino acid Nos. 21 to 129 in SEQ ID NO: 21 (FIG. 18) in the Sequence Listing. The nucleotide sequence of the light chain constant region of MAb1 corresponds to the nucleotides Nos. 388 to 705 in SEQ ID NO: 20 (FIG. 17) in the Sequence Listing. The amino acid sequence thereof corresponds to amino acid Nos. 130 to 234 in SEQ ID NO: 21 (FIG. 18) in the Sequence Listing. The amino acid sequence of DCRH1 is described in SEQ ID NO: 22 (FIG. 23). The amino acid sequence of CDRH2 is described in SEQ ID NO: 23 (FIG. 23). The amino acid sequence of CDRH3 is described in SEQ ID NO: 24 (FIG. 23). The amino acid sequence of CDRL1 is described in SEQ ID NO: 25 (FIG. 23). The amino acid sequence of CDRL2 is described in SEQ ID NO: 26 (FIG. 23). The amino acid sequence of CDRL3 is described in SEQ ID NO: 27 (FIG. 23).

Antibody 2 (MAb2) is a monoclonal antibody obtained by the intraperitoneal administration of the RX protein purified from the culture supernatant of ADSF cells to a BALB/c mouse according to the method described in paragraph c) of Example 2.

The nucleotide sequence and amino acid sequence of the heavy chain variable region of MAb2 are described in SEQ ID NOs: 32 (FIG. 19) and 33 (FIG. 20), respectively, in the Sequence Listing. The nucleotide sequence and amino acid sequence of the light chain variable region of MAb2 are described in SEQ ID NOs: 34 (FIG. 21) and 35 (FIG. 22), respectively, in the Sequence Listing. The amino acid sequence of CDRH1 is described in SEQ ID NO: 36 (FIG. 23). The amino acid sequence of CDRH2 is described in SEQ ID NO: 37 (FIG. 23). The amino acid sequence of CDRH3 is described in SEQ ID NO: 38 (FIG. 23). The amino acid sequence of CDRL1 is described in SEQ ID NO: 39 (FIG. 23). The amino acid sequence of CDRL2 is described in SEQ ID NO: 40 (FIG. 23). The amino acid sequence of CDRL3 is described in SEQ ID NO: 41 (FIG. 23).

Antibody 3 (Mab3) is a monoclonal antibody obtained by the intraperitoneal administration of the RX protein purified from the culture supernatant of ADSF cells to a BALB/c mouse according to the method described in paragraph c) of Example 2.

The nucleotide sequence and amino acid sequence of the heavy chain variable region of MAb3 are described in SEQ ID NOs: 62 (FIG. 30) and 68 (FIG. 31), respectively, in the Sequence Listing. The nucleotide sequence and amino acid sequence of the light chain variable region of MAb3 are described in SEQ ID NOs: 64 (FIG. 32) and 65 (FIG. 33), respectively, in the Sequence Listing. The amino acid sequence of CDRH1 is described in SEQ ID NO: 66 (FIG. 23), The amino acid sequence of CDRH2 is described in SEQ ID NO: 67 (FIG. 23). The amino acid sequence of CDRH3 is described in SEQ ID NO: 68 (FIG. 23). The amino acid sequence of CDRL1 is described in SEQ ID NO: 69 (FIG. 23). The amino acid sequence of CDRL2 is described in SEQ ID NO: 70 (FIG. 23). The amino acid sequence of CDRL3 is described in SEQ ID NO: 71 (FIG. 23).

Antibody 4 (MAb4) is a monoclonal antibody obtained by the intraperitoneal administration of ADSF cells to a BALB/c mouse according to the method described in Example 13.

The nucleotide sequence and amino acid sequence of the heavy chain variable region of MAb4 are described in SEQ ID NOs: 108 (FIG. 75) and 109 (FIG. 76), respectively, in the Sequence Listing. The nucleotide sequence and amino acid sequence of the light chain variable region of MAb4 are described in SEQ ID NOs: 110 (FIG. 77) and 111 (FIG. 78), respectively, in the Sequence Listing. The amino acid sequence of CDRH1 is described in SEQ ID NO: 112 (FIG. 79). The amino acid sequence of CDRH2 is described in SEQ ID NO: 113 (FIG. 70). The amino acid sequence of CDRH3 is described in SEQ ID NO: 114 (FIG. 79). The amino acid sequence of CDRL1 is described in SEQ ID NO: 115 (FIG. 79). The amino acid sequence of CDRL2 is described in SEQ ID NO: 116 (FIG. 79). The amino acid sequence of CDRL3 is described in SEQ ID NO; 117 (FIG. 79).

The antibody mutant of the present invention preferably exhibits, for example, reduced sensitivity to protein degradation or oxidation, an improved biological activity, an improved ability to bind to the antigen, or physicochemical or functional properties imparted thereto. Examples of such an antibody mutant can include an antibody having an amino acid sequence derived from the amino acid sequence of the original antibody by conservative amino acid substitution. The conservative amino acid substitution is substitution that occurs in an amino acid group related to amino acid side chains.

Preferable amino acid groups are as follows: an acidic group including aspartic acid and glutamic acid; a basic group including lysine, arginine, and histidine; a nonpolar group including alanine, valine, leucine, isoleucine, proline, phenylalanine, methionine, and tryptophan; and an uncharged polar family including glycine, asparagine, glutamine, cysteine, serine, threonine, and tyrosine. Other preferable amino acid groups are as follows: an aliphatic hydroxy group including serine and threonine; an amide-containing group including asparaginic and glutamine; an aliphatic group including alanine, valine, leucine, and isoleucine; and an aromatic group including phenylalanine, tryptophan, and tyrosine. Such amino acid substitution in the antibody mutant is preferably performed without reducing the antigen binding activity of the original antibody.

The present invention also encompasses, for example: an antibody mutant having an amino acid sequence derived from the amino acid sequence of MAb1, MAb2, Mab3, or MAb4 of the present invention by conservative five amino acid substitution; and a mouse antibody, a rat antibody, a chimeric antibody, a humanized antibody, or a human antibody comprising a CDR having an amino acid sequence in which a conservative amino acid mutation occurs in the amino acid sequence of any of CDRH1 to CDRH3 and CDRL1 to CDRL3 derived from MAb1, and MAb2, MAb3, and/or MAb4.

The constant regions of the antibody of the present invention are not particularly limited. Preferably, constant regions derived from a human antibody are used in the antibody of the present invention for the treatment or prevention of a disease in a human. Examples of the heavy chain constant region of the human antibody can include Cγ1, Cγ2, Cγ3, Cγ4, Cμ, Cδ, Cα1, Cα2, and Cε. Examples of the light chain constant region of the human antibody can include Cκ and Cλ.

The monoclonal antibody of the present invention that is used in the treatment or prevention of a human disease is preferably a chimeric antibody having the constant regions of a human antibody, a humanized antibody, or a human anti-body, more preferably a humanized antibody or a human antibody.

### (3-6) Detection of RX Protein Using Antibody

The antibody of the present invention may recognize the RX protein present in human autoimmune disease. The antibody of the present invention also includes, for example, an antibody that recognizes the RX protein expressed in the joint tissue of an RA patient. Exemplary partial amino acid sequences of the RA patient-derived RX protein are shown in Nos. 1 to 4 of FIG. 12 (SEQ ID NOs: 56 to 59 in the Sequence Listing), but are not limited to those sequences.

### (3-7) Functional Fragment of Antibody

According to one aspect, the present invention provides a functional fragment of the anti-RX antibody of the present invention. The functional fragment of an antibody means a fragment of the antibody of the present invention that maintains at least a portion of the functions of the antibody. Examples of such functions of the antibody can generally include an antigen binding activity, an antigen activity-regulating activity, an antibody-dependent cytotoxic activity, and a complement-dependent cytotoxic activity. Examples of the functions of the anti-RX antibody of the present invention can include an RX protein binding activity, and an anti-arthritic activity, i.e., a suppressive activity against the action of the RX protein of causing the onset and/or exacerbation of arthritis in the joint and/or a suppressive activity against bone destruction.

The functional fragment of an antibody is not particularly limited as long as the fragment of the antibody maintains at least a portion of the activities of the antibody. Examples thereof can include, but are not limited to, Fab, E(ab′)2, Fv, single chain Fv (scFv) comprising heavy and light chain Fvs linked via an appropriate linker, diabodies, linear antibodies, polyspecific antibodies formed from antibody fragments, and Fab′, which is a monovalent fragment of antibody variable regions obtained by the treatment of F(ab′)2 under reducing conditions.

A molecule that is derived from the antibody protein by the deletion of 1 to several or more amino acid(s) at its amino terminus and/or carboxy terminus and maintains at least a portion of the functions of the antibody is also encompassed in the meaning of the functional fragment of the antibody. For example, the heavy chain of an antibody produced by cultured mammalian cells is known to lack a lysine residue at the carboxy terminus (Journal of Chromatography A, 705: 129-134 (1995)). Also, the heavy chain of such an antibody is known to lack two amino acid residues (glycine and lysine) at the carboxy terminus and instead have an amidated proline residue at the carboxy terminus (Analytical Biochemistry, 360: 75-83 (2007)). The deletion and the modification in these heavy chain sequences, however, do not influence the ability of the antibody to bind to the antigen or other functions or its effector functions (complement activation, antibody-dependent cytotoxic action, etc.). such a modified form of the functional fragment of the antibody is also encompassed by the antibody of the present invention or the functional fragment thereof, or a modified form (described later) of the antibody or the functional fragment.

The antibody of the present invention or the functional fragment thereof may be a polyspecific antibody having specificity for at least 2 types of different antigens. The polyspecific antibody is not limited to a bispecific antibody, which binds to 2 types of different antigens, and an antibody having specificity for 3 or more types of different antigens is also encompassed in the meaning of the “polyspecific antibody” of the present invention.

The polyspecific antibody of the present invention may be a full-length antibody or a functional fragment thereof (e.g., bispecific F(ab′)2 antibody). The bispecific antibody can also be prepared by linking the heavy and light chains (HL pairs) of two types of antibodies. Alternatively, the bispecific antibody may be obtained by fusing two or more types of monoclonal antibody-producing hybridomas to prepare bispecific antibody-producing fusion cells (Millstein et al., Nature (1983) 305, p, 537-539). The polyspecific antibody can also be prepared in the same way as above.

According to one aspect, the antibody of the present invention is a single chain antibody (single chain Fv; hereinafter, referred to as “scFv”). The scFv is obtained by linking the heavy and light chain V regions of the antibody via a polypeptide linker (Pluckthun, The Pharmacology of Monoclonal Antibodies, 113 (Rosenburg and Moore, ed.), Springer Verlag, New York, p. 269-315 (1994); and Nature Biotechnology (2005), 23, p. 1126-1136). Also, bi-scFv comprising two scFvs linked via a polypeptide linker can be used as a bispecific antibody. Alternatively, multi-scFv comprising three or more scFvs may be used as a polyspecific antibody.

The present invention includes a single chain immunoglobulin comprising full-length heavy and light chain sequences or the antibody linked via an appropriate linker (Lee, H-S, et al., Molecular Immunology (1999), 36, p. 61-71; and Shirrmann, T. et al., mAbs (2010), 2 (1) p. 1-4). Such a single chain immunoglobulin can be dimerized to thereby maintain a structure and activities similar to those of the antibody, which is originally a tetramer. Also, the antibody of the present invention may be an antibody that has a single heavy chain variable region and was no light chain sequence. Such an antibody, called a single domain antibody (sdAb) or a nanobody, has been reported to maintain the ability to bind to an antigen (Muyldemans S. et al., Protein Eng. (1994), 7 (9), 1129-35; and Hamers-Casterman C. et al., Nature (1993), 363 (6429), 446-8). These antibodies are also encompassed in the meaning of the functional fragment of the antibody according to the present invention.

### (5-8) Humanized Antibody

The antibody of the present invention also includes a humanized antibody. Examples of the humanized antibody of the present invention can include, but are not limited to, a human-derived antibody having CDRs replaced with the CDRs of a non-human animal antibody (see Nature (1986), 321, p. 522-525), a human antibody grafted with the CDR sequences and with some amino acid residues of framework regions by CDR grafting (see WO90/07861 and 086972323), and an antibody having human antibody amino acid(s) replaced for one or two or more non-human animal antibody-derived amino acid(s) in any of these humanized antibodies.

Examples of the anti-RX humanized antibody or a functional fragment thereof can include an antibody that consists of a heavy chain comprising CDRH1 consisting of the amino acid sequence represented by SEQ ID NO: 22 in the Sequence Listing, CDRH2 consisting of the amino acid sequence represented by SEQ ID NO: 23 in the Sequence Listing, and CDRH3 consisting of the amino acid sequence represented by SEQ ID NO: 24 in the Sequence Listing, and a light chain comprising CDRL1 consisting of the amino acid sequence represented by SEQ ID NO: 25 in the Sequence Listing, CDRL2 consisting of the amino acid sequence represented by SEQ ID NO: 26 in the Sequence Listing, and CDRL3 consisting of the amino acid sequence represented by SEQ ID NO: 27 in the Sequence Listing, and that recognizes the RX protein of the present invention, and a fragment of the antibody that maintains the RX protein binding activity of the antibody.

Examples of such a humanized antibody can include: an antibody whose heavy chain variable region comprises a peptide represented by an amino acid sequence described in any one of SEQ ID NOs: 82 to 86 (FIGS. 40 to 49) in the Sequence Listing and whose light chain variable region comprises a peptide represented by an amino acid sequence described in any one of SEQ ID NOs: 82 to 86 (FIGS. 50 to 54) in the Sequence Listing, and a functional fragment thereof; and an antibody whose heavy chain variable region is a peptide represented by an amino acid sequence described in any one of SEQ ID NOs: 72 to 81 (FIGS. 40 to 49) in the Sequence Listing and whose light chain variable region is a peptide represented by an amino acid sequence described in any one of SEQ ID NOs: 82 to 86 (FIGS. 50 to 54) in the Sequence Listing, and a functional fragment thereof.

In the present invention, heavy chains whose variable region is a peptide represented by an amino acid sequence described in any one of SEQ ID NOs: 72 to 81 (FIGS. 40 to 49) in the Sequence Listing and whose constant region is derived from human IgG1 are referred to as H1 to H10, respectively. In the present invention, light chains whose variable region is a peptide represented by an amino acid sequence described in any one of SEQ ID NOs: 81 to 86 (FIGS. 50 to 54) in the Sequence Listing and whose constant region is derived from human IgG1 are referred to as L1 to L5, respectively. In the present invention, an antibody having the heavy chain H1 and the light chain L1 is referred to as T1; an antibody having the heavy chain H1 and the light chain L2 is referred to as T2; an antibody having the heavy chain H1 and the light chain L3 is referred to as T3; an antibody having the heavy chain H2 and the light chain L1 is referred to as T4; an antibody having he heavy chain H2 and the light chain L2 is referred to as T5; an antibody having the heavy chain H2 and the light chain L3 is referred to as T6; an antibody having the heavy chain H2 and the light chain L5 is referred to as T7; an antibody having the heavy chain H3 and the light chain L1 is referred to as T8; an antibody having the heavy chain H3 and the light chain L2 is referred to as T9; an antibody having the heavy chain H3 and the light chain L3 is referred to as T10; an antibody having the heavy chain H3 and the light chain L5 is referred to as T11; an antibody having the heavy chain H4 and the light chain L4 is referred to as T12; an antibody having the heavy chain H5 and the light chain L1 is referred to as T13; an antibody having the heavy chain H5 and the light chain L2 is referred to as T14; an antibody having the heavy chain H5 and the light chain L5 is referred to as T15; an antibody having the heavy chain H6 and the light chain L2 is referred to as T16; an antibody having the heavy chain H6 and the light chain L5 is referred to as T17; an antibody having the heavy chain H7 and the light chain L2 is referred to as T18; an antibody having the heavy chain H8 and the light chain L1 is referred to as T19; an antibody having the heavy chain H9 and the light chain L1 is referred to as T20; and an antibody having the heavy chain H10 and the light chain L1 is referred to as T21.

The humanized antibody T1 consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region, and has a high binding activity against the RX protein.

The humanized antibody T2 consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein.

The humanized antibody T3 consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein.

The humanized antibody T3 consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region, and has a high binding activity against the RX protein.

The humanized antibody T4 consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 82 (FIG. 80) in the Sequence Listing and a human IgG1-derived constant region, and has a high binding activity against the RX protein.

The humanized antibody T5 consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein, an in vivo anti-arthritic function, and a chemokine production inhibitory function.

The humanized antibody T6 consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region, and has a high binding activity against the RX protein.

The humanized antibody T7 consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID No: 86 (FIG. 54) in the Sequence Listing and a human IgG1-derived constant region, and has a high binding activity against the RX protein.

The humanized antibody T8 consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region, and has a high binding activity against the RX protein, an in vivo anti-arthritic function, and inhibitory function of inflammatory cytokine and chemokine production.

The humanized antibody T9 consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 83 (FIG. 51) in the Sequence Listing and a human IqG1-derived constant region, and has a high binding activity against the RX protein and an in vivo anti-arthritic function.

The humanized antibody T10 consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region, and has a high binding activity against the RX protein.

The humanized antibody T11 consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region, and has a high binding activity against the RX protein.

The humanized antibody T12 consists of a heavy chain having a variable region consisting of the amino acid sequence represented by SEQ ID NO: 75 (FIG. 53) in the Sequence Listing and a human IgG1-derived constant region, and a light chain having a variable region consisting of the amino acid sequence represented by SEQ ID NO: 85 (FIG. 53) in the Sequence Listing and a human IgG1-derived constant region, and has a high binding activity against the RX protein.

The humanized antibody T13 consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region, and has a high binding activity against the RX protein, an in vivo anti-arthritic function, and inhibitory function of inflammatory cytokine and chemokine production.

The humanized antibody T14 consists of a heavy chain having a variable region consisting of the amino acid sequence and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein, an in vivo anti-arthritic function, and inhibitory function of inflammatory cytokine and chemokine production.

The humanized antibody T15 consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region, and has a high binding activity against the RX protean.

The humanized antibody T16 consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein.

The humanized antibody T17 consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 36 (FIG. 54) in the Sequence Listing and a human IgG1-derived constant region, and has a high binding activity against the RX protein.

The humanized antibody T18 consists of a heavy chain having a variable region consisting of the amino acid sequence represented by SEQ ID NO: 78 (FIG. 46) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region, and has a high binding activity against the RX protein.

The humanized antibody T19 consists of a heavy chain having a variable region consisting of the amino acid sequence represented by SEQ ID NO: 79 (FIG. 47) in the Sequence Listing and a human IgG1-derived constant region, and a light chain having a variable region consisting of the amino acid sequence represented by SEQ ID NO: 82 (FIG. 80) in the Sequence Listing and a human IgG1-derived constant region, and has a high binding activity against the RX protein.

The humanized antibody T20 consists of a heavy chain having a variable region consisting of the amino acid sequence represented by SEQ ID NO: 80 (FIG. 48) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region, and has a high binding activity against the RX protein.

The humanized antibody T21 consists of a heavy chain having a variable region consisting of the amino acid sequence represented by SEQ I NO: 81 (FIG. 49) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region, and has a high binding activity against the RX protein.

The anti-RX humanized antibody of the present invention is preferably T1 to T21, more preferably T3 to T15 and T18, even more preferably T8 to T11, T13 to T15, and T18, further more preferably T13 to T15.

A collagen-induced arthritis mouse model that received T5, T8, T9, T13, or T14 showed clinical findings similar to those of an untreated normal mouse without exhibiting weight change, autonomic disturbance (piloerection), abnormal posture, abnormal gait, decline in locomotor activity, respiratory abnormality, irritable urination, salivation, lacrimation, exophthalmos, ataxia of limbs or general ataxia, etc.

Other examples of the anti-RX humanized antibody or a functional fragment thereof can include an antibody that consists of a heavy chain comprising CDRH1 consisting of the amino acid sequence represented by SEQ ID NO: 36 in the Sequence Listing, CDRH2 consisting of the amino acid sequence represented by SEQ ID NO: 37 in the Sequence Listing, and CDRH3 consisting of the amino acid sequence represented by SEQ ID NO: 38 in the Sequence Listing, and a light chain comprising CDRL1 consisting of the amino acid sequence represented by SEQ ID NO: 39 in the Sequence Listing, CDRL2 consisting of the amino acid sequence represented by SEQ ID NO: 40 in the Sequence Listing, and CDRL3 consisting of the amino acid sequence represented by SEQ ID NO: 41 in the Sequence Listing, and that recognizes the RX protein of the present invention, and a fragment of the antibody that maintains the RX protein binding activity of the antibody.

Alternative examples of the anti-RX humanized antibody or a functional fragment thereof can include an antibody that consists of a heavy chain comprising CDRH1 consisting of the amino acid sequence represented by SEQ ID NO: 66 in the Sequence Listing, CDRH2 consisting of the amino acid sequence represented by SEQ ID NO: 67 in the Sequence Listings and CDRH3 consisting of the amino acid sequence represented by SEQ ID NO: 68 in the Sequence Listing, and a light chain comprising CDRL1 consisting of the amino acid sequence represented by SEQ ID NO: 69 in the Sequence Listing, CDRL2 consisting of the amino acid sequence represented by SEQ ID NO: 70 in the Sequence Listing, and CDRL3 consisting of the amino acid sequence represented by SEQ ID NO: 71 in the Sequence Listing, and that recognizes the RX protein of the present invention, and a fragment of the antibody that maintains the RX protein binding activity of the antibody.

Further alternative examples of the anti-RX humanized antibody or a functional fragment thereof can include an antibody that consists of a heavy chain comprising CDRH1 consisting of the amino asset sequence represented by SEQ ID NO: 112 in the Sequence Listing, CDRH2 consisting of the amino acid sequence represented by SEQ ID NO: 113 in the Sequence Listing, and CDRH3 consisting of the amino acid sequence represented by SEQ ID NO: 114 in the Sequence Listing, and a light chain comprising CDRL1 consisting of the amino acid sequence represented by SEQ ID NO: 115 in the Sequence Listing, CDRL2 consisting of the amino acid sequence represented by SEQ ID NO: 116 in the Sequence Listing, and CDRL3 consisting of the amino acid sequence represented by SEQ ID NO: 117 in the Sequence Listing, and that recognizes the REX protein of the present invention, and a fragment of the antibody that maintains the RX protein binding activity of the antibody.

The present invention also encompasses an antibody that comprises a heavy or light chain comprising an amino acid sequence 80% or higher, 82% or higher, 84% or higher, 86% or higher, 83% or higher, 90% or higher, 92% or higher, 94% or higher, 96% or higher, 98% or higher, or 99% or higher identical to the amino acid sequence of the heavy or light chain of any one of the antibodies MAb1 to MAb4 of the present invention, the chimeric antibodies thereof, and the humanized antibodies thereof (including T1 to T21) and binds to the RX protein, or a functional fragment thereof. This sequence identity is preferably 95% or higher, more preferably 96% or higher, even more preferably 97% or higher, further more preferably 98% or higher, most preferably 99% or higher. Such an antibody preferably has one or more of the activities described in paragraph (3-3).

The identity or homology between two types of amino acid sequences can be determined using the default parameter of Blast algorithm version 2.2.2 (Altschul, Stephen F., Thomas L. Madden, Alejandro A. Schaffer, Jinghui Zhang, Zheng Zhang, Webb Miller, and David J. Lipman (1997), “Gapped BLAST and PSi-BLAST: a new generation of protein database search programs”, Nucleic Acids Res. 25:3389-3402). The Blast algorithm is also available, for example, by Internet access at http://blast.ncbi.nlm.nih.gov/.

The present invention also encompasses an antibody that comprises a heavy or light chain comprising an amino acid sequence derived from the amino acid sequence of the heavy or light chain of any one of the antibodies MAb1 to MAb4 or the present invention, the chimeric antibodies thereof, and the humanized antibodies thereof (including T1 to T21) by the substitution, deletion, addition, or insertion of 1 to 50, 1 to 45, 1 to 40, 1 to 35, 1 to 30, 1 to 25, 1 to 20, 1 to 15, 1 to 10, 1 to 8, 1 to 6, 1 to 5, 1 to 4, 1 to 3, 1 or 2, or 1 amino acid(s) and binds to the RX protein, or a functional fragment thereof. This amino acid mutation is preferably substitution. The number of mutated amino acids is preferably 1 to 5, more preferably 1 to 4, even more preferably 1 to 3, further more preferably 1 to 2, most preferably 1. Such an antibody preferably has one or more of the activities described in paragraph (3-3).

The present invention also encompasses an antibody that comprises a heavy or light chain comprising an amino acid sequence encoded by the nucleotide sequence of a nucleic acid hybridizing under stringent conditions to a nucleic acid having a nucleotide sequence complementary to a nucleotide sequence encoding the amino acid sequence of the heavy or light chain of any one of the antibodies MAb1 to MAb4 of the present invention, the chimeric antibodies thereof, and the humanized antibodies thereof (including T1 to T21), and that binds to the RX protein, or a functional fragment thereof. Such an antibody preferably has one or more of the activities described in paragraph (3-3).

The anti-RX humanized antibody of the present invention or the functional fragment thereof has an anti-arthritic activity, preferably 30% or more anti-arthritic activity, more preferably 50% or more anti-arthritic activity.

The humanized antibody of the present invention has a KD value of 1×10⁻⁷ or lower, preferably 1×10⁻⁸ or lower more preferably 5×10⁻⁹ or lower, even more preferably 2×10⁻⁹ or lower, further more preferably 1×10⁻⁹ or lower, most preferably 5×10⁻¹⁰ or lower for the RX protein.

### (3-9) Antibody Binding to Same Site

An “antibody binding to the same site” as in the case of the antibody provided by the present invention is also included in the antibody of the present invention. The “antibody binding to the same site” as in the case of a certain antibody means another antibody that binds to a site on an antigen molecule recognized by the antibody. If a second antibody binds to a partial peptide or a partial three-dimensional structure on an antigen molecule bound by a first antibody, the first and second antibodies can be determined to bind to the same site. Alternatively, the first and second antibodies can be determined to bind to the same site by confirming that the second antibody competes with the first antibody for binding to the antigen, i.e., the second antibody interferes with the binding of the first antibody to the antigen, even if the peptide sequence or three-dimensional structure of the specific binding site is not determined. When the first and second antibodies bind to the same site and the first antibody has an effect characteristic of one aspect of the antibody of the present invention, such as an anti-arthritic activity, the second antibody also has an exceedingly high probability of having the same activity thereas. Thus, if a second anti-RX antibody binds to a site bound by a first anti-RX antibody, the first and second antibodies can be determined to bind to the same site on the RX protein. Alternatively, the first and second anti-RX antibodies can be determined to bind to the same site on the RX protein by confirming that the second anti-RX antibody competes with the first anti-RX antibody for binding to the RX protein.

The present invention also encompasses: an antibody binding to a site on the RX protein recognized by any one of MAb1, MAb2, MAb3, and MAb4 of the present invention, the chimeric antibodies thereof, the humanized antibodies thereof, the functional fragments of these antibodies, and modified forms of the antibodies or the functional fragments; a functional fragment thereof; or a modified form of the antibody or the functional fragment.

MAb2, and its chimerized antibodies and humanized antibodies, recognize the 10th to 16th amino acids PPILHPV counted from the amino terminus of the amino acid sequence (FIG. 25: SEQ ID NO: 15) of gp73ED. This partial amino acid sequence is also found in the amino acid sequence of gp52SU.

MAb3, and its chimerized antibodies and humanized antibodies, recognize a partial amino acid sequence in the amine acid sequence (FIG. 25: SEQ ID NO: 15) of gp73ED. The partial amino acid sequence is also found in the amino acid sequence of gp52SU.

MAb4, and its chimerized antibodies and humanized antibodies, recognize gp28ED.

An antibody that competes with any one of the antibodies MAb1 to MAb4 for binding to the antigen is also included in the antibody of the present invention. Such an antibody preferably has one or more of the activities described in paragraph (3-3).

The antibody binding site can be determined by a method well known by those skilled in the art, such as immunoassay. For example, a series of antigen fragment peptides are prepared by appropriately sequentially cleaving the amino acid sequence of the antigen from its C terminus or N terminus, and the reactivity of the antibody thereto is studied to roughly determine a recognition site. Then, shorter peptides are synthesized, and the reactivity of the antibody to these peptides can be studied to thereby determine the binding site. The antigen fragment peptides can be prepared using a technique such as gene recombination, protease digestion, or peptide synthesis.

When the antibody binds to or recognizes the partial conformation of the antigen, the binding site for the antibody can be determined by identifying amino acid residues on the antigen adjacent to the antibody using X-ray structural analysis, site-directed mutagenesis experiments, deuterium exchange NMR, deuterium exchange mass spectrometry, etc.

MAb1, and its chimerized antibodies and humanized antibodies (including T1 to T21), recognize the partial conformation of gp52SU. The partial conformation is also found in gp73 ED.

### (3-10) Modified Form of Antibody or Functional Fragment Thereof

In one aspect, the present invention provides a modified form of the antibody or the functional fragment thereof. The modified form of the antibody of the present invention or the functional fragment thereof means an antibody of the present invention or a functional fragment thereof provided with chemical or biological modification. The chemically modified form includes, for example, a form having an amino acid skeleton conjugated with a chemical moiety, and a form having a chemically modified N-linked or O-linked carbohydrate chain. The chemically modified form may contain a toxic, or cytotoxic portion. The biologically modified form includes, for example, a form that has undergone post-translational modification (e.g., N-linked or O-linked glycosylation or N-terminal or C-terminal processing), and a form containing a methionine residue added to the N-terminus by expression using prokaryotic host cells. such a modified form is also meant to include a form labeled to permit detection or isolation of the antibody or the antigen of the present invention, for example, an enzyme-labeled form, a fluorescently labeled form, or an affinity-labeled form. Such a modified form of the antibody of the present invention or the functional fragment thereof is useful for improvement of the stability or retention in blood circulation of the original antibody of the present invention or the original functional fragment thereof, reduction in antigenicity, detection or isolation of the antibody or the antigen, etc.

Example of the chemical moiety contained in the chemically modified form can include water-soluble polymers such as polyethylene glycol, ethylene glycol/propylene glycol copolymers, carboxymethylcellulose, dextran, and polyvinyl alcohol.

Examples of the chemical moiety contained in the chemically modified form can include water-soluble polymers such as polyethylene glycol, ethylene glycol/propylene glycol copolymers, carboxymethylcellulose, dextran, and polyvinyl alcohol.

Examples of the biologically modified form can include a form modified by enzymatic treatment, cell treatment, or the like, a form fused with another peptide, such as a tag, added by gene recombination, and a form prepared from host cells expressing an endogenous or exogenous sugar chain-modifying enzyme.

The antibody-dependent cellular cytotoxic activity of the antibody of the present invention or the functional fragment thereof may be enhanced by adjusting the modification (glycosylation, defucosylation, etc.) of the sugar chain bound with the antibody or the functional fragment. For example, methods described in WO99/54342, WO00/61739, and WO92/31140are known as such a technique of adjusting the sugar chain modification of the antibody, though this technique is not limited thereto. The modified form of the antibody of the present invention also includes an antibody that has undergone the sugar chain modification thus adjusted.

Such a modification may be made at an arbitrary position or a desired position in the antibody or the functional fragment thereof. Alternatively, the same or two or more different modifications may be made at one or two or more positions therein.

The modified form of the antibody of the present invention or the functional fragment thereof has excellent stability and has, for example, high thermal stability and a neutral to weakly basic isoelectric point.

In the present invention, the “modified form of the antibody fragment” is also meant to include even a “fragment of the modified form of the antibody”.

In the present invention, the modified form of the antibody or the modified form of the functional fragment thereof is also simply referred to as an “antibody” or a “functional fragment of the antibody”. The “antibody” (modified form) of any one of MAb1 to MAb4, chimerized MAb1 to MAb4, and humanized MAb1 to MAb4 (including T1 to T21) also includes, for example, an antibody that lacks carboxy-terminal 1 to several amino acids in its heavy or light chain or has a modified carboxy-terminal amino acid(s) in its heavy or light chain.

### 4. Method for Producing Antibody

### (4-1) Method Using Hybridoma

In order to prepare the anti-RX antibody of the present invention, anti-RX antibody-producing cells are isolated from the spleens of animals immunized with the RX protein according to the method of Kohler and Milstein (Kohler and Milstein, Nature (1975), 256, p. 495-497; and Kennet, R. ed., Monoclonal Antibodies, p. 365-367, Plenum Press, N.Y. (1980)). The cells are fused with myeloma cells to thereby establish hybridomas. Monoclonal antibodies can be obtained from cultures of these hybridomas.

### (4-1-1) Preparation of Antigen

The antigen for the preparation of the anti-RX antibody can be obtained according to, for example, the method for preparing native or recombinant RX protein described in other paragraphs of the present specification. Examples of the antigen that may be thus prepared can include the RX protein or an RX protein fragment comprising a partial sequence with at least 6 consecutive amino acids of the RX protein, and their derivatives further comprising an arbitrary amino acid sequence or carrier added thereto (hereinafter, collectively referred to as an “RX antigen”).

The recombinant RX antigen can be prepared by transfecting host cells with a gene comprising a nucleotide sequence encoding the amino acid sequence of the RX antigen, and collecting the antigen from cultures of the cells. The native RX antigen can be purified or isolated from, for example, human or rodent tissues with arthritis, cells derived from the tissues, or cultures of the cells. An RX antigen obtained in a cell-free in vitro translation system from a gene comprising a nucleotide sequence encoding the amino acid sequence of the RX antigen is also included in the “RX antigen” of the present invention.

### (4-1-2) Production of Anti-RX Monoclonal Antibody

The monoclonal antibody is typically produced through the following steps:

(a) preparing an antigen,

(b) preparing antibody-producing cells,

(c) preparing myeloma cells (hereinafter, referred to as “myelomas”),

(d) fusing the antibody-producing cells with the myelomas,

(e) screening for a hybridoma group producing the antibody of interest, and

(f) obtaining single cell clones (cloning).

This production method further invoices (g) a step of culturing the hybridomas, a step of raising hybridoma-transplanted animals, etc, and (h) a step of assaying or determining the biological activity of the monoclonal antibody, etc., if necessary.

Hereinafter, the method for preparing the monoclonal antibody will be described in detail with reference to these steps. However, the method for preparing the antibody is not limited to those steps, and, for example, antibody-producing cells other than spleen cells and myelomas may be used.

### (a) Step of Preparing Antigen

This step is performed according to the method for preparing the RX protein described above in (2-3) .

### (b) Step of Preparing Antibody-Producing Cell

The antigen obtained in step (a) is mixed with an adjuvant such as a complete or incomplete Freund's adjuvant or potassium aluminum sulfate, and laboratory animals are immunized with the resulting immunogen. Any laboratory animal used in a hybridoma preparation method known in the art can be used without limitations. Specifically, for example, mice, rats, goats, sheep, cattle, or horses can be used. From the viewpoint of readily available myeloma cells to be fused with isolated antibody-producing cells, etc., the animals to be immunized are preferably mice or rats.

The strain of mice or rats actually used is not particularly limited. In the case of mice, for example, A, AKR, BALB/c, BDP, BA CE, C3H, 57BL, C57BL, C57L, DBA, FL, HTH, HT1, LP, NZB, NZW, RF, R III, SJL, SWR, WB, or 129 can be used. In the case of rats, for example, Wistar, Low, Lewis, Sprague-Dawley, ACI, BN, or Fischer can be used.

These mice and rats are available from laboratory animal breeders or distributors, for example, CLEA Japan, Inc. or Charles River Laboratories Japan Inc.

Of those mice and rats, a BALB/c mouse strain or Wistar and Low rat strains are particularly preferable as animals to be immunized in consideration of fusion compatibility with the myeloma cells described later.

Also, in consideration of the homology between human and mouse antigens, mice whose biological mechanism to remove autoantibodies has been reduced, i.e., autoimmune disease mice, are also preferably used.

In this context, these mice or rats are preferably 5 to 12 weeks old, more preferably 6 to 8 weeks old, at the time of immunization.

The animals can be immunized with the RX protein using, for example, the method of Weir, D. M., Handbook of Experimental Immunology, Vols. I, II, and III, Blackwell Scientific Publications, Oxford (1987); and Kabat, E. A. and Mayer, M. M., Experimental Immunochemistry, Charles C Thomas Publisher Springfield, Ill. (1964).

Examples of method for determining antibody does can include, but are not limited to, immunoassay such as RIA and ELISA.

Antibody-producing cells derived from spleen cells or lymphocytes separated from the immunized animals can be prepared according to a method known in the art, for example, the method of Kohler et al., Nature (1975), 256, p. 495; Kohler et al. , Eur. J. Immunol. (1977), 6, p. 511; and Milstein et al., Nature (1977), 266, p. 550; and Walsh, Nature (1977), 266, p. 495.

In the case of spleen cells, a general method can be adopted, which involves chopping the spleens, filtering cells through a stainless mesh, and then floating the resulting cells in an Eagle's minimum essential median (MEM) or the like to separate antibody-producing cells.

### (c) Step of Preparing Myeloma

The myeloma cells used in cell fusion are not particularly limited and can be selected appropriately for use from cell lines known in the art. For example, a hypozanthine-guanine phosphoribosyl transferase (HGPRT)-deficient line, i.e., mouse-derived X63-Ag8 (X63), NS1-ANS/1 (NS1), P3X63-Ag8.U1 (P301), X63-Ag8.653 (X63.653), SP2/0-Ag14 (SP2/0), MPC11-45.6TG1.7 (45.6TG), FO, 3149/5XXO, or BU.1, rat-derived 210.RSY3.Ag1.2.3 (Y3), or human-derived U266AR (SKO-007), GM1500-GTG-A12 (GM1500), UC729-6, LICR-LOW-HMy2 (HMy2), or 8226AR/NIP4-1 (NP41), whose screening procedures have already been established, is preferably used in consideration of convenience in the selection of hybridomas from fusion cells. These HGPRT-deficient lines are available from, for example, American Type Culture Collection (ATCC).

These cell lines are subcultured in an appropriate medium, for example, an 8-azaguanine medium [RPMI-1640 medium supplemented with glutamine, 2-mercaptoethanol, gentamicin, and fetal calf serum (hereinafter, referred to as “FCS”) and further supplemented with 8-azaguanine], an Iscove's modified Dulbecco's medium (hereinafter, referred to as “IMDM”), or a Dulbecco's modified Eagle medium (hereinafter, referred to as “DMEM”) and subcultured in a normal medium (e.g., ASF104 medium (manufactured by Ajinomoto Co., Inc.) containing 10% FCS) 3 to 4 days before cell fusion to secure the number of cells equal to or greater than 2×10⁷ cells on the day of cell fusion.

(d) Step of Fusing Antibody-Producing Cell with Myeloma Cell

The antibody-producing cells can be fused with the myeloma cells under conditions that present cell viability from being exceedingly reduced, according to any method known in the art (e.g., Weir, D. M., Handbook of Experimental Immunology, Vols. I, II, and III, Blackwell Scientific Publications, Oxford (1987); and Kabat, E. A. and Mayer, M. M., Experimental Immunochemistry, Charles C Thomas Publisher Springfield, Ill. (1964)). For example, a chemical method which involves mixing antibody-producing cells with myeloma cells in a high-concentration solution of a polymer such as polyethylene glycol, or a physical method using electric stimulation can be used.

### (e) Step of Screening for Hybridoma Group Producing Antibody of Interest

A method for selection from the hybridomas obtained by cell fusion is not particularly limited, and a hypoxanthine-aminopterin-thymidine (HAT) selection method (Kohler et al., Nature (1975), 256, p. 495; and Milstein et al., Nature (1977), 266, p. 550) is typically used. This method is effective for obtaining hybridomas using an HGPRT-deficient myeloma cell line, which cannot survive in the presence of aminopterin. Specifically, unfused cells and hybridomas can be cultured in HAT medium to thereby allow only hybridomas resistant to aminopterin to selectively live and grow.

### (f) Step of Obtaining Single Cell Clone (Cloning)

The hybridomas can be cloned using any method known in the art, for example, a methylcellulose, soft agarose, or limiting dilution method (see e.g., Barbara, B. M. and Stanley, M. S.: Selected Methods in Cellular Immunology, W. H. Freeman and Company, San Francisco (1980)). The limiting dilution method is preferable.

### (g) Step of Culturing Hybridoma and Step of Raising Hybridoma-Transplanted Animal

The selected hybridomas can be cultured to thereby produce monoclonal antibodies. Preferably, the desired hybridomas are cloned and then subjected to antibody production.

The monoclonal antibody produced by such a hybridoma can be collected from cultures of the hybridoma. Also, a recombinant antibody can be collected from cultures of cells transfected with the monoclonal antibody gene. Alternatively, the hybridoma may be injected intraperitoneally to mice of the same strain (e.g., BALB/c described above) or Nu/Nu mice and allowed to grow. Then, the monoclonal antibody can be collected from their ascites.

### (h) Step of Assaying or Determining Biological Activity of Monoclonal Antibody

Various biological tests can be selected and applied thereto according to the purpose.

### (4-2) Cell Immunization Method

Cells expressing the native RX protein, cells expressing the recombinant RX protein or its fragment, or the like can be used as immunogens to thereby prepare an anti-RX antibody by the hybridoma method described above.

The cells expressing the native RX protein can be found in cells derived from animals with experimentally induced arthritis or from the tissues of patients affected with autoimmune disease such as RA or arthritis. Such cells are preferably mouse-derived cells, but are not limited to them. Examples of such mouse-derived cells can include the ADSF cells of the present invention. These mouse-derived cells expressing the RX protein are used in an remount of 1×10⁵ to 1×10⁹ cells, preferably 1×10⁶ to 1×10⁸ cells, more preferably 0.5 to 2×10⁷ cells, even more preferably 1×10⁷ cells, per shot. The number of cells subjected to immunization can be changed according to the expression level of the RX protein. The immunogens are generally administered intraperitoneally and may be administered through an intradermal route or the like. The hybridomas can be prepared by the application of the method described in paragraphs (4-1-2).

### (4-3) Gene Recombination

In order to prepare the antibody of the present invention, host cells are transfected with a nucleic acid (heavy chain nucleic acid) comprising a nucleotide sequence encoding the amino acid sequence of its heavy chain and a nucleic acid (light chain nucleic acid) comprising a nucleotide sequence encoding the amino acid sequence of its light chain, or with a vector containing an insert of the heavy chain nucleic acid and a vector containing an insert of the light chain nucleic acid, and then cultured, and the antibody can be collected from the cultures. The heavy chain nucleic acid and the light chain nucleic acid may be inserted in one vector.

Examples of the nucleic acid comprising a nucleotide sequence encoding the amino acid sequence of the heavy chain variable region of the present invention (heavy chain variable region nucleic acid) can include nucleic acids of H1 to H10. Examples of the nucleotide sequences of the nucleic acids of H1 to H10 can include nucleotide sequences described in SEQ ID NOs: 91 to 100 (FIGS. 58 to 67), respectively, in the Sequence Listing. Examples of the heavy chain variable region nucleic acid can also include a nucleic acid that hybridizes under stringent conditions to a nucleic acid comprising a nucleotide sequence complementary to any of these nucleotide sequences and comprises a nucleotide sequence encoding the amino acid sequence of the humanized antibody heavy chain variable region of the present invention.

Examples of the nucleic acid comprising a nucleotide sequence encoding the amino acid sequence of the light chain variable region of the present invention (light chain variable region nucleic acid) can include nucleic acids of L1 to L5. Examples of the nucleotide sequences of the nucleic acids of L1 to L5 can include nucleotide sequences described in SEQ ID NOs: 103 to 107 (FIGS. 69 to 73), respectively, in the Sequence Listing. Examples of the light chain variable region nucleic acid can also include a nucleic acid that hybridizes under stringent conditions to a nucleic acid comprising a nucleotide sequence complementary to any of these nucleotide sequence and comprises a nucleotide sequence encoding the amino acid sequence of the humanized antibody light chain variable region of the present invention.

Prokaryotic or eukaryotic cells can be used as host cells. In the case of using host eukaryotic cells, animal cells, plant cells, or eukaryotic microbes can be used.

Examples of the animal cells can include mammal-derived cells, i.e., monkey-derived COS cells (Gluzman, Y. Cell (1981), 23, p. 175-182, ATCC CRL-1650), mouse fibroblast NIH3T3 (ATCC No. CRL-1658), Chinese hamster ovary cells (CHO cells, ATCC CCL-61), dihydrofolate reductase-deficient lines thereof (CHO^{dhfx-}; Urlaub, C. and Chasin, L. A. Proc. Natl. Acad. Sci. U.S.A. (1980), 77, p. 4126-4220), cells derived from birds such as chickens, and cells derived from insects.

Also, cells modified by the modification of their sugar chain structures to enhance the biological activity of the antibody can be used as hosts. For example, CHO cells modified such that sugar chains free from fucose bound to N-acetylglucosamine at their reducing ends account for 20% of complex-type N-glycoside-linked sugar chains binding to the Fc region of the antibody can be used to thereby prepare an antibody having an enhanced ADCC or CDC activity (WO02/31140).

Examples of the eukaryotic microbes can include yeasts.

Examples of the prokaryotic cells can include E. coli and Bacillus subtilis.

A signal peptide for the secretion of the antibody of the present invention (monoclonal antibody derived from each animal, rat antibody, mouse antibody, chimerized (chimeric) antibody, humanized antibody, human antibody, etc.) is not limited to the secretory signal of an antibody of the same species, the same type, and the same subtype as the antibody of the present invention or to the own secretory signal of the antibody of the present invention. Any secretory signal of an antibody of different type or subtype therefrom or any secretory signals or a protein derived from a different eukaryotic species therefrom or a prokaryotic species can be selected and used.

In the present invention, the heavy and light chains of a mature antibody, a functional fragment thereof, a modified form of the antibody or the functional fragment obtained by the preparation method such as gene recombination are usually free from a signal peptide in most cases. The light and/or heavy chains may contain a portion or the whole of the signal peptide.

### (4-4) Method for Preparing Human Antibody

Further examples of the antibody of the present invention can include a human antibody. The anti-RX human antibody means an anti-RX antibody consisting of the amino acid sequence of a human-derived antibody. The anti-EX human antibody can be obtained by a method using human antibody-producing mice carrying human genomic DNA fragments comprising human antibody heavy and light chain genes (see e.g., Tomiruka, K. et al., Nature Genetics (1997), 16, p. 133-143; Kuroiwa, Y. et al., Nuc. Acids Res. (1998), 26, p. 3447-3448; Yoshida, H. et al., Animal Cell Technology: Basic and Applied Aspects vol. 10, p. 69-73 (Kitagawa, Y., Matsuda, T. and Iijima, S. eds.), Kluwer Academic Publishers, 1999; and Tomizuka, K. et al., Proc. Natl. Acad. Sci. USA (2000), 97, p. 722-727).

Specifically, such transgenic animals may be any recombinant animals that are obtained by disrupting the endogenous immunoglobulin heavy and light chain gene loci of non-human mammals and instead introducing thereto human immunoglobulin heavy and light chain gene loci via yeast artificial chromosome (YAC) vectors or the like, and animals that are created by crossing these animals.

Alternatively, eukaryotic cells may be transformed with cDNAs encoding the heavy and light chains, respectively, of such a human antibody, preferably with vectors comprising the cDNAs by a gene recombination technique. The transformed cells producing a recombinant human monoclonal antibody are cultured. This antibody can be obtained from the culture supernatant. The own secretory signal of the antibody as well as any other secretory signal, for example, the secretory signal of an antibody of different class or subclass therefrom or derived from different species therefrom, or any eukaryote- or prokaryote-derived secretory protein can be used.

In this context, for example, eukaryotic cells, preferably mammalian cells such as CHO cells, lymphocytes, or myelomas, can be used as hosts.

Also, a method for obtaining a phage display-derived human antibody selected from a human antibody library (see e.g., Wormstone, I. M. et al., Investigative Ophthalmology & Visual Science (2002), 43 (7), p. 2301-2308; Carmen, S. et al., Briefings in Functional Genomics and Proteomics (2002), 1 (2), p. 189-203; and Siriwardens, D. et al., Ophthalmology (2002), 109 (3), p. 427-431) is known.

For example, a phage display method (Nature Biotechnology (2005), 23, (9), p. 1105-1116) can be used, which involves allowing the variable regions of a human antibody to be expressed as a single chain antibody (scFv) on phage surface and selecting a phage binding to the antigen.

The phage selected on the basis of its ability to bind to the antigen can be subjected to gene analysis to thereby determine DNA sequences encoding the variable regions of the human antibody binding to the antigen.

If the DNA sequence of scFv binding to the antigen is determined, an expression vector having this sequence can be prepared and introduced to appropriate hosts to allow them to express the human antibody (WO92/01047, WO92/20791, WO93/06213, WO93/11236, WO93/19172, WO95/01438, WO95/15388, Annu. Rev. Immunol (1994), 12, p. 433-455; and Nature Biotechnology (2005), 23(9), p. 1105-1116).

### (4-5) Method for Preparing Functional Fragment of Antibody

The method for preparing a single chain antibody is well known in the art (see e.g., U.S. Pat. Nos. 4,946,778, 5,260,203, 5,091,513, and 5,455,030). in this scFv, a heavy chain variable region and a light chain variable region are linked via a linker that prevents them from forming a conjugate, preferably a polypeptide linker (Huston, J. S. et al., Proc. Natl. Acad. Sci. U.S.A. (1988), 85, p. 5879-5883), The heavy chain variable region and the light chain variable region in scFv may be derived from the same antibody or may be derived from different antibodies.

For example, an arbitrary single chain peptide consisting of 12 to 19 residues is used as the polypeptide linker that links there variable regions.

In order to obtain scFv-encoding DNA, of the sequences of DNA encoding the heavy chain or heavy chain variable region of the antibody and DNA encoding the light chain or light chain variable region thereof, each DNA portion encoding the whole or desired amino acid sequence is used as a template and amplified by PCR using a printer pair flanking both ends of the template. Subsequently, DNA encoding the polypeptide linker moiety is further amplified in combination with a printer pair flanking both ends of the DNA so that the obtained fragment can be linked at its ends to the heavy and light chain DNAs, respectively.

The scFv-encoding DNA can be used to thereby prepare, according to a routine method, an expression vector containing the DNA and host cells transformed with the expression vector. In addition, the cost cells are cultured, and the scFv can be collected from the cultures according to a routine method.

Also in order to obtain any other functional fragment of the antibody, a gene encoding the functional fragment is obtained according to the method described above and introduced into cells. The functional fragment of interest can be collected from cultures of the cells.

The antibody of the present invention may be multimerized to thereby enhance its affinity for the antigen. In this case, antibodies of the same type may be multimerized, or a plurality of antibodies recognizing a plurality of epitopes, respectively, of the same antigen may be multimerized. Examples of methods for multimerizing these antibodies can include the binding of two scFvs to an IgG CH3domain, the binding thereof to streptavidin, and the introduction of a helix-turn-helix motif.

The antibody of the present invention may be a mixture of plural types of anti-RX antibodies differing in amino acid sequence, i.e., a polyclonal antibody. Examples of the polyclonal antibody can include a mixture of plural types of antibodies differing in a portion or the whole of CDRs. Such a polyclonal antibody can be collected from cultures of mixed-cultured different antibody-producing cells (WO2004/061104). Alternatively, separately prepared antibodies may be mixed. Antiserum, which is one aspect of the polyclonal antibody, can be prepared by immunizing animals with the desired antigen and collecting serum from the animals according to a routine method.

Antibodies conjugated with various molecules such as polyethylene glycol (PEG) can also be used as modified forms of the antibody.

The antibody of the present invention may further be any conjugates formed by these antibodies with other drugs (immunoconjugates). Examples of such an antibody can include the antibody conjugated with a radioactive material or a compound having a pharmacological action (Nature Biotechnology (2005), 23, p. 1137-1116).

### (4-6) Purification of Antibody

The obtained antibody can be purified until homogeneous. Usual protein separation and purification methods can be used for the separation and purification of the antibody.

The antibody can be separated and purified by appropriately selected or combined approach(es), for example, chromatography columns, filters, ultrafiltration, salting out, dialysis, preparative polyacrylamide gel electrophoresis, and/or isoelectric focusing (Strategies for Protein Purification and Characterization: A Laboratory Course Manual, Daniel R, Marshak et al, eds., Cold Spring Harbor Laboratory Press (1996); and Antibodies: A Laboratory Manual. Ed Harlow and David Lane, Cold Spring Harbor Laboratory (1988)), though the separation and purification method is not limited thereto.

Examples of chromatography include affinity chromatography, ion-exchange chromatography, hydrophobic chromatography, gel filtration, reverse-phase chromatography, and adsorption chromatography.

These chromatography approaches can be performed using liquid-phase chromatography such as HPLC or FPLC.

Examples of columns used in affinity chromatography can include protein A, protein G, and antigen columns.

Examples of commercially available protein A columns include Protein A ceramic HyperD F (Pall Corp.), POROS (R) Protean A (Applied Biosystems, Inc.), Mabselect, Protein A Sepharose F.F. (GE Healthcare Bio-Sciences Corp.), and Prosep rA and Prosep A (Millipore Corp.)

Also, the antibody may be purified using its binding activity against the antigen using an antigen-immobilized carrier.

### (4-7) Gene, Vector, and Cell

The present invention provides a gene encoding the antibody of the present invention or the functional fragment thereof, or the modified form of the antibody or the functional fragment (hereinafter, this gene is referred to as an “antibody gene”), a recombinant vector containing an insert of the gene, a cell containing the gene or the vector introduced therein (hereinafter, this cell is referred to as an “antibody gene-introduced cell”), and a cell producing the antibody of the present invention (hereinafter, this cell is referred to as an “antibody-producing cell”).

Preferably, the antibody gene of the present invention comprises any one of the following nucleotide sequences (a) to (e) (hereinafter, each is referred to as an “antibody gene sequence”), consists of a nucleotide sequence comprising the antibody gene sequence, or consists of the antibody gene sequence:

(a) a combination of a nucleotide sequence encoding the heavy chain amino acid sequence of any one of the antibodies MAb1 to MAb4 of the present invention, the chimeric antibodies thereof, and the humanized antibodies thereof (including T1 to T21) and a nucleotide sequence encoding the light chain amino acid sequence of any one of thereof;

(b) a combination of a nucleotide sequence encoding the amino acid sequence of a heavy chain comprising CDRH1 to CDRH3 of any one of the antibodies MAb1 to MAb4 of the present invention, the chimeric antibodies thereof, and the humanized antibodies thereof (including T1 to T21) and a nucleotide sequence encoding the amino acid sequence of a light chain comprising CDRL1 to CDRL3 of any one thereof;

(c) a combination of a nucleotide sequence encoding a heavy chain amino acid sequence comprising the amino acid sequence of the heavy chain variable region of any one of the antibodies MAb1 to MAb4 of the present invention, the chimeric antibodies thereof, and the humanized antibodies thereof (including T1 to T21) and a nucleotide sequence encoding a light chain amino acid sequence comprising the amino acid sequence of the light chain variable region of any one thereof;

(d) a nucleotide sequence that hybridizes under stringent conditions to a nucleic acid consisting of a nucleotide sequence complementary to any one of the nucleotide sequences (a) to (c) and encodes the amino acid sequence of an antibody binding to the RX protein; and

(e) a nucleotide sequence that comprises a nucleotide sequence derived from any one of the nucleotide sequences (a) to (c) by the substitution, deletion, addition, or insertion of 1 to 50, 1 to 45, 1 to 40, 1 to 30, 1 to 25, 1 to 20, 1 to 15, 1 to 10, 1 to 8, 1 to 6, 1 to 5, 1 to 4, 1 to 3, 1 or 2, or 1 base(s) and encodes the amino acid sequence of an antibody binding to the RX protein.

The antibody having the amino acid sequence encoded by the nucleotide sequence (d) or (e) may have one or two or more of the activities described in paragraph (3-3), in addition to an RX protein binding activity.

However, the antibody gene of the present invention is not limited to those described in (a) to (e).

The present invention provides, as described in paragraph (4-3), a method for producing the antibody of the present invention or the functional fragment thereof, or the modified form of the antibody or the functional fragment, comprising the steps of: culturing the antibody gene-introduced cell of the present invention and collecting the antibody, the functional fragment, or the modified form from the cultures. The antibody or the functional fragment thereof, or the modified form of the antibody or the functional fragment obtained by this production method is also included in the present invention.

### 5. Pharmaceutical Composition

The present invention provides a pharmaceutical composition comprising the anti-RX antibody or the functional fragment thereof, or the modified term of the antibody or the functional fragment.

The pharmaceutical composition of the present invention is useful in the treatment or prevention of autoimmune disease or arthritis, particularly these diseases in an individual expressing the RX protein. The term “autoimmune disease” means a disease that exhibits certain symptoms in response to factors (including cells, tissues, etc) of the self by the immune system supposed to serve as a mechanism of body's defense against the invasion of a foreign object. Examples of an autoimmune disease can include rheumatoid arthritis (RA), systemic lupus erythematosus, Sjogren's syndrome, Crohn's disease, psoriasis, collagen disease, generalized scleroderma, cryoglobulinemia causes by systemic lupus erythematosus, etc., polymyositis, and dermatomyositis. Examples of RA can include narrowly defined RA, systemic-onset juvenile rheumatoid arthritis (Still's disease), and adult-onset Still's disease (Still's disease that is developed in an adult). The term “arthritis” means a disease accompanied by joint inflammation. Examples of symptoms of arthritis can include joint redness, swellings tenderness or pain (arthralgia), rigidity, local warmth, impaired motor functions, fever, general malaise, and weight loss. Arthritis includes, for example, acute monoarthritis, acute polyarthritis, chronic monoarthritis/arthropathy, and chronic polyarthritis.

Examples of acute monoarthritis can include bacterial arthritis and gout. Examples of acute polyarthritis can include viral polyarthritis. Examples of chronic monoarthritis/arthropathy can include non-inflammatory and inflammatory diseases such as osteoarthritis and traumatic arthritis. Examples of chronic polyarthritis can include RA and psoriatic arthritis. In the present invention, arthritis includes juvenile idiopathic arthritis (hereinafter, referred to as “JIA”). Examples of JIA can include generalized arthritis, RF-negative polyarthritis, RF-positive polyarthritis, oligoarthritis, and psoriatic arthritis. In the present invention, the treatment and/or prevention of a disease includes, but is not limited to, the prevention of the onset of the disease, preferably the disease in an individual expressing the RX protein, the suppression or inhibition of exacerbation or progression thereof, the alleviation of one or two or more symptoms exhibited by an individual affected with the disease, the suppression or remission of exacerbation or progression thereof, the treatment or prevention of a secondary disease, etc.

The pharmaceutical composition of the present invention can contain a therapeutically or prophylactically effective amount of the anti-RX antibody or the functional fragment of the antibody and a pharmaceutically acceptable diluent, vehicle, solubilizer, emulsifier, preservative, and/or additive.

The “therapeutically or prophylactically effective amount” means an amount that exerts therapeutic or prophylactic effects on a particular disease by means of a particular dosage form and administration route.

The pharmaceutical composition of the present invention may contain materials for changing, maintaining, or retaining pH, osmotic pressure, viscosity, transparency, color, tonicity, sterility, or the stability, solubility, sustained release, absorbability, permeability, dosage form, strength, properties, shape, etc., of the composition or the antibody contained therein (hereinafter, referred to as “pharmaceutical materials”). The pharmaceutical materials are not particularly limited as long as the materials are pharmacologically acceptable. For example, no or low toxicity is a property preferably possessed by these pharmaceutical materials.

Examples of the pharmaceutical materials can include, but are not limited to, the following: amino acids such as glycine, alanine, glutamine, asparaginic, histidine, arginine, and lysine; antimicrobial agents; antioxidants such as ascorbic acid, sodium sulfate, and sodium bisulfite; buffers such as phosphate, citrate, or borate buffers, sodium bicarbonate, and Tris-HCl solutions; fillers such as mannitol and glycine; chelating agents such as ethylenediaminetetraacetic acid (EDTA); complexing agents such as caffeine, polyvinylpyrrolidine, β-cyclodextrin, and hydroxypropyl-β-cyclodextrin; bulking agents such as glucose, mannose, and dextrin; other hydrocarbons such as monosaccharides, disaccharides, glucose, mannose, and dextrin; coloring agents; corrigents; diluents; emulsifiers; hydrophilic polymers such as polyvinylpyrrolidine; low-molecular-weight polypeptides; salt-forming counterions; antiseptics such as benzalkonium chloride, benzoic acid, salicylic acid, thimerosal, phenethyl alcohol, methylparaben, propylparaben, chlorhexidine, sorbic acid, and hydrogen peroxide; solvents such as glycerin, propylene glycol, and polyethylene glycol; sugar alcohols such as mannitol and sorbitol; suspending agents; surfactants such as PEG, sorbitan ester, polysorbates such as polysorbate 20 and polysorbate 60, triton, tromethamine, lecithin, and cholesterol; stability enhancers such as sucrose and sorbitol; elasticity enhancers such as sodium chloride, potassium chloride, mannitol, and sorbitol; transport agents; diluents; excipients; and/or pharmaceutical additives.

The amount of these pharmaceutical materials is 0.001 to 1000 times, preferably 0.01 to 100 times, more preferably 0.1 to 10 times the weight of the anti-TX antibody or the functional fragment thereof, or the modified form of the antibody or the functional fragment.

An immunoliposome comprising the anti-RX antibody or the functional fragment thereof, or the modified form of the antibody or the functional fragment encapsulated in a liposome, or a modified antibody form comprising the antibody conjugated with a liposome (U.S. Pat. No. 6,214,388, etc.) is also included in the pharmaceutical composition of the present invention.

The excipients or vehicles are not particularly limited as long as they are liquid or solid materials usually used in injectable water, saline, artificial cerebrospinal fluids, and other preparations for oral or parenteral administration. Examples of saline can include neutral saline and serum albumin-containing saline.

Examples of buffers can include a Tris buffer solution adjusted to bring about the final pH of the pharmaceutical composition to 7.0 to 8.5, an acetate buffer solution adjusted to bring about the final pH thereof to 4.0 to 5.5, a citrate buffer solution adjusted to bring about the final pH thereof to 5.0 to 8.0, and a histidine buffer solution adjusted to bring about the final pH thereof to 5.0 to 8.0.

The pharmaceutical composition of the present invention is a solid, a liquid, a suspension, or the like. Another example of the pharmaceutical composition of the present invention can include freeze-dried preparations. The freeze-dried preparations can be formed using an excipient such as sucrose.

The administration route of the pharmaceutical composition of the present invention may be any of enteral administration, local administration, and parenteral administration. Examples thereof can include intravenous administration, intraarterial administration, intramuscular administration, intradermal administration, hypodermic administration, intraperitoneal administration, transdermal administration, intraosseous administration, intraarticular administration, and the like.

The recipe for the pharmaceutical composition can be determined according to the administration method, the binding affinity of the antibody for the RX protein, etc. The anti-RX antibody of the present invention or the functional fragment thereof, or the modified form of the antibody or the functional fragment having higher affinity (lower KD value) for the RX protein can exert its pharmaceutical efficacy at a lower dose.

The dose of the anti-RX antibody of the present invention can be determined appropriately according to the species of an individual, the type of disease, symptoms, sex, age, pre-existing conditions, the binding affinity of the antibody for the RX protein or its biological activity, and other factors. A dose of usually 0.01 to 1000 mg/kg, preferably 0.1 to 100 mg/kg, can be administered once every day to every 180 days or twice or three or more times a day.

Examples of the form of the pharmaceutical composition can include injections (including freeze-dried preparations and drops), suppositories, transnasal absorption preparations, transdermal absorption preparations, sublingual formulations, capsules, tablets, ointments, granules, aerosols, pills, powders, suspensions, emulsions, eye drops, and biological implant formulations.

The pharmaceutical composition comprising the anti-RX antibody or the functional fragment thereof, or the modified form of the antibody or the functional fragment as an active ingredient can be used in combination with additional therapeutic or prophylactic agent(s) selected from DMARDs, steroid drugs, and/or nonsteroidal anti-inflammatory drugs (NSAIDs). The pharmaceutical composition and the additional therapeutic or prophylactic agent(s) can be administered concurrently or sequentially. For example, the pharmaceutical composition comprising the anti-RX antibody or the functional fragment thereof, or the modified form of the antibody or the functional fragment as an active ingredient is administered after administration of DMARD, a steroid drug, and/or NSAID or before administration of DMARD, a steroid drug, and/or NSAID. Alternatively, the pharmaceutical composition and DMARD, a steroid drug, and/or NSAID may be administered concurrently. Examples of DMARD can include MTX. Also, the pharmaceutical composition of the present invention may be administered as an alternative drug or a concomitant drug to a patient that receives the administration of an anti-TNFα agent, an anti-IL-1 agent, an anti-IL-6 agent, CTLA4-Ig, an anti-CD20 antibody, a JAK inhibitor, or the like.

The present invention provides even a method for treating or preventing autoimmune disease such as RA or arthritis, use of the antibody of the present invention for preparing a pharmaceutical composition for treatment or prevention of autoimmune disease such as RA or arthritis, and use of the antibody of the present invention for treating or preventing autoimmune disease such as RA or arthritis. The present invention also includes a kit for treatment or prevention comprising the antibody of the present invention.

### 6. Composition for Diagnosis

The present invention provides a composition for examination or diagnosis comprising the anti-RX antibody of the present invention or the functional fragment thereof, or the modified form of the antibody or the functional fragment (hereinafter, collectively referred to as a “composition for diagnosis”). The antibody, the functional fragment, or the modified form contained in the composition for diagnosis of the present invention is not particularly limited as long as it binds to the RX protein.

The composition for diagnosis of the present invention is useful in the examination or diagnosis of autoimmune diseases such as RA or arthritis. The composition for diagnosis of the present invention is also useful in the examination or diagnosis of early RA or pre-RA symptoms, which do not satisfy the conventional diagnosis criteria, undiagnosed arthritis (UA) that evolves to RA, etc. In the present invention, the examination or the diagnosis includes, for example, the determination or examining of a risk of developing a disease, the determination of the presence or absence of a disease, the examining of the degree of progression or exacerbation, the examining or determination of the effect of drug therapy using the pharmaceutical composition comprising the anti-RX antibody or the like, the examining or determination of the effect of therapy other than drug therapy, the examining of a risk of recurrence, and the determination of the presence or absence of recurrence. However, the examination or the diagnosis according to the present invention is not limited to these, and any approach can be used.

When the RX protein is detected in a 2, 3, 4, 5, 7, 8, 9, 10, 11, 12, 13, 14, 15, 16, 17, 18, 19, or 20-fold or more amount in a sample derived from a test subject compared with a sample derived from a healthy individual, the test subject can be diagnosed as having rheumatoid arthritis or as being at a high risk of developing it. When the concentration of the RX protein in blood exceeds a particular reference value, the test subject is diagnosed as having RA or can be diagnosed as being at a high risk of developing RA.

The composition for diagnosis can contain a pH buffer, an osmoregulator, salts, a stabilizer, an antiseptic, a color developer, a sensitizer, an aggregation inhibitor, and the like.

The present invention provides a method for examining or diagnosing autoimmune disease such as RA, arthritis, or the like, use of the antibody of the present invention for preparing a composition for diagnosis of autoimmune disease such as RA, arthritis, or the like, and use of the antibody of the present invention for examining or diagnosing autoimmune disease such as RA, arthritis, or the like. The present invention also includes a kit for examination or diagnosis comprising the antibody of the present invention. This kit can contain the RX protein or a fragment thereof, or a modified form of the protein or the fragment as a standard.

The desirable examination or diagnosis method involving the antibody of the present invention is sandwich ELISA. Any usual detection method using antibodies, such as ERISA, RIA, enzyme-linked immunospot (ELISPOT) assay, dot blotting, Ouchterlony test, or counterimmunoelectrophoresis (CIE), may be used. Antibodies applied to the sandwich ERISA assay system may be any combination of two types of antibodies that recognize MMTV env, but do not compete with each other. The antibodies can be labeled by a method using biotin or by any other labeling method that can be carried out in biochemical analysis using a labeling material such as HRP, alkaline phosphatase, or FITC. A chromogenic substrate seen as 3,3′,5,5′-tetramethylbenzidine (TMB), 5-bromo-4-chloro-3-indolyl phosphate (BCIP), ρ-nitrophenyl phosphate (ρ-NPP), o-phenylenediamine (OPD), 3-ethylbenzothiazoline-6-sulfonic acid (ABTS), SuperSignal ELISA Pico Chemiluminescent Substrate (Thermo Fisher Scientific Inc.), a fluorescent substrate such as QuantaBlu™ Fluorogenic Peroxidase Substrate (Thermo Fisher Scientific Inc.), and a chemiluminescent substrate can be used in direction using enzymatic labeling. Sample derived from human or non-human animals as well as artificially treated sampler such as recombinant proteins can be subjected to this assay. Examples of test samples derived from individual organisms can include, but are not limited to, blood, synovial fluids, ascites, lymph, cerebrospinal fluids, and tissue homogenate supernatants. Examples of the blood sample can include, but are not limited to, serum and plasma.

The sandwich ELISA kit for examination or diagnosis comprising the antibody of the present invention may contain a solution of RX protein standards, a coloring reagent, a buffer solution for dilution, an antibody for solid phase, antibody for detection, and a washing solution, and the like. The amount of the antibody bound to the antigen can be measured preferably by the application of a method such as an absorbance, fluorescence, luminescence, or radioisotope (RI) method. An absorbance plate reader, a fluorescence plate reader, a luminescence plate reader, an RI liquid scintillation counter, or the like is preferably used in the measurement.

The present invention provides a method for detecting the RX protein and a method for quantifying the Rx protein. These methods each comprise the step of contacting a test sample with the anti-RX antibody. The present invention also encompasses a reagent comprising the anti-RX antibody. The detection method, the quantification method, and the reagent may be used for the examination and/or diagnosis described above.

### EXAMPLES

Hereinafter, the present invention will be described specifically with reference to Examples. However, the present invention is not intended to be limited to them.

The procedures of the Examples below were performed according to the methods described in “Molecular Cloning” (Sambrook, J., Fritsch, E. F. and Maniatis, T., Cold Spring Harbor Laboratory Press, 1989) or the methods described in other experimental manuals used by those skilled in the art, or using commercially available reagents or kits according to the instruction manuals, unless otherwise specified.

### Example 1

### Establishment of Cell Line Involved in Exacerbation of Arthritis

a) Establishment of Autonomously Growing Cell Line from Joint of Collagen-Induced Arthritis Mouse Model

A cell line involved in the exacerbation of arthritis was established from the joint of an arthritis mouse model as follows: the arthritis was induced according to the method described in T. S. Courtensy, Nature, 283, 666, 1980. Specifically, an emulsion of bovine type II collagen (Collagen Gijutsu Kenshukai Y.K.) and a complete Freund's adjuvant was intradermally administered to the tail head of each male DBA/1 mouse (Charles River Laboratories Japan Inc.). Two weeks later, an emulsion of bovine type II collagen and an incomplete Freund's adjuvant was intradermally administered thereto in the same way as above to cause collagen-induced arthritis. The malleolar joint tissue of a hindlimb was aseptically collected from a mouse with serious arthritis, and cut finely in a culture dish. The tissue slices were cultured in a culture medium (RPMI1640 medium supplemented with 10% FCS) to extract cells from the tissue slices. While the state of the extracted cells was observed under a microscope, half the amount of the culture medium was replaced with fresh medium every 3 to 7 days. A sufficient amount of the extracted cells was confirmed and then dissociated by trypsin treatment to collect the cells and the tissue slices from the dish. Unnecessary tissue slices were filtered off through a 70-μm cell strainer (Becton, Dickinson and Company). The cells that passed through the strainer were continuously cultured with half the amount of the medium replaced with fresh medium every 3 to 7 days. After stabilization of cell growth, the culture medium containing 10% FCS was gradually replaced with a serum-free medium. In this way, an autonomously growing cell line was established from the joint of the collagen-induced arthritis mouse model (hereinafter, this cell line is referred to as “ADSF cells”).

### b) Confirmation of Function of ADSF Cell on Exacerbation of Arthritis

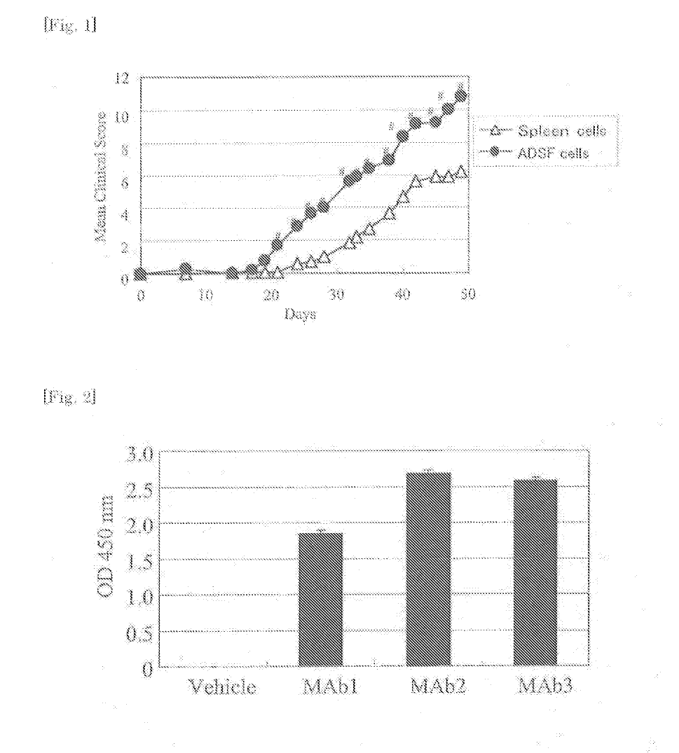

In order to study the relationship of ADSF cells to arthritis, the ADSF cells themselves were intraperitoneally administered to a collagen-induced arthritis mouse model and evaluated for their influence on the exacerbation of arthritis. The collagen-induced arthritis was caused by intradermally administering an emulsion of bovine type II collagen and a complete Freund's adjuvant to the tail head of each male DBA/1 mouse two times at a 14-day interval. Groups each involving 10 mice were divided into an ADSF cell-administered group and a mouse spleen cell-administered group as a negative control. On×10⁷ cells were intraperitoneally administered to each mouse 7 times at 7-day intervals from the day of the first sensitization with collagen. The exacerbation of arthritis was evaluated on the basis of the total score of limbs by scoring the degree of arthritis in each limb on a scale of 0 to 4 (5 stages). The arthritis scores of the ADSF cell-administered group and the mouse spleen cell-administered group were subjected to a significance test by the Mann-Whitney U-test method. Results showed that the arthritis score of the ADSF cell-administered group was significantly higher than that of the mouse spleen cell-administered group at day 21 or later after the sensitization with collagen, demonstrating that the administration or ADSF cells significantly exacerbated arthritis (FIG. 1, #: p<0.05).

### Example 2

### Preparation of Monoclonal Antibody Having Anti-Arthritis Function

a) Preparation of Monoclonal Antibody using ADSF Cell Tab Concentrated Solution of Culture Supernatant Thereof

For the purpose of obtaining an antibody that suppresses the exacerbation of arthritis, 1×10⁷ ADSF cells and a concentrated solution of their culture supernatant were mixed and intraperitoneally and intradermally administered to each WKY/NCrj rat and one of its soles, respectively. Booster immunization was performed to enhance the antibody titer. Three days after final immunization, lymph nodes were collected, and cells were isolated and fused by the addition of a myeloma cell line 8-653 at a cell number ratio of 1:7 according to a routine method. Polyethylene glycol (molecular weight: 4000) heated in advance to 37° C. was added as a cell fusion promoter at a final concentration of 35% (w/v). The cell fusion was completed by mild centrifugation (900 rpm, within 5 minutes). Then, the cells were resuspended by the addition of medium and centrifuged to collect cells, which were then screened for fusion cells using a HAT selective medium, (containing hypoxanthine, aminopterin, and thymidine). Next, the obtained fusion cells were prepared as single clones by limiting dilution analysis and then screened for fusion cells (hybridomas) producing antibodies binding to the culture supernatant components of ADSF cells. Specifically, the antibodies were reacted with a microplate with immobilized culture supernatant components of ADSF cells. Subsequently, horseradish peroxidase (hereinafter, referred to as “HRP”)—labeled anti-rat secondary antibodies were reacted therewith. TMB substrates (MP Biomedicals, LLC (Cappel)) were allowed to emit color. The reaction was terminated with 0.1 N hydrochloric acid, and the absorbance was measured at a wavelength of 450 nm using a plate reader (Multiscan Biochromatic; Labsystems Diagnostics Group). Results showed that Monoclonal Antibody 1 (MAb1) binding to the culture supernatant components of ADSF cells was obtained. This antibody was purified according to a routine method using a Protein G affinity column from the ascites of a nude mouse that intraperitoneally received the hybridoma producing the antibody.

### b) Analysis of Antigen Recognized by Antibody

### 1) Purification of Recognized Antigen

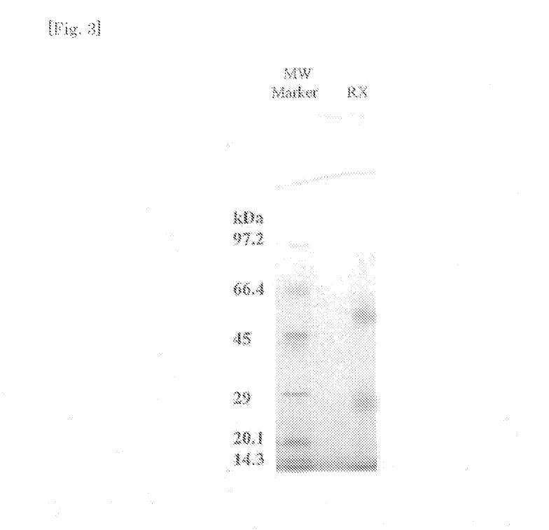

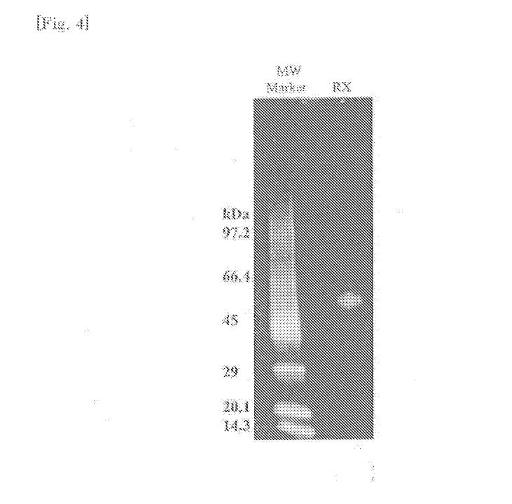

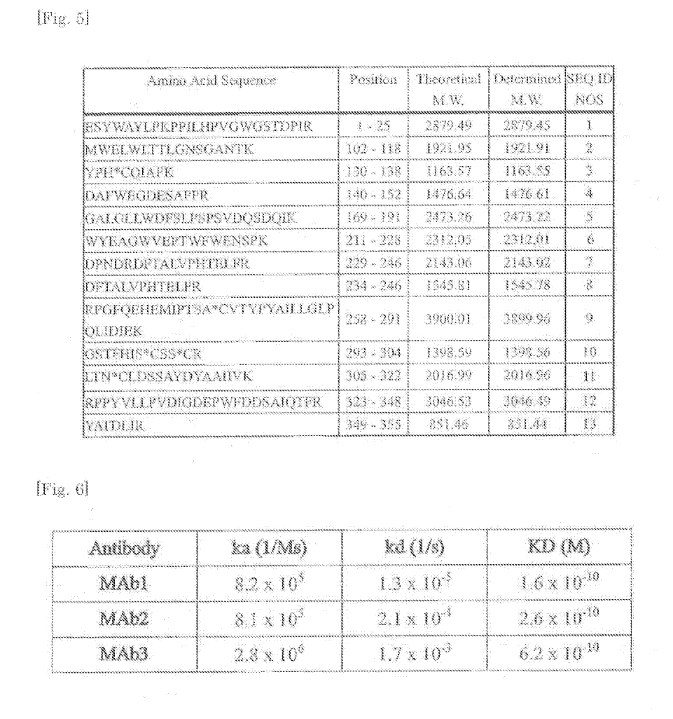

An antigen recognized by MAb1 was purified as follows: MAb1 prepared according to paragraph a) of Example 2 was cross-linked with an affinity gel carrier (Immunopure Immobilized Protein G Plus gel; Thermo Fisher Scientific K.K.) using a coupling reagent (disuccinimidyl suberate) to prepare an antibody column. The culture supernatant of ADSF cells cultured in a CL-1000 flask was added to this antibody column. The column was washed with sterilized PBS in an amount of 10 times the volume of the column, followed by elation with a 100 mM glycine-HCl buffer solution (pH 2.8). The eluate was immediately neutralized with a 1 M tris-HCl buffer solution (pH 9.0) in an amount of 1/10 of the volume of the solution. The solution was concentrated using an ultrafiltration filter (Centriprep MWCO10K; Millipore Corp.). The buffer was replaced with PBS. The purified antigen protein was analyzed by sodium dodecyl sulfate-polyacrylamide gel electrophoresis (hereinafter, referred to as “SDS-PAGE”) under non-reducing conditions. As a result, bands with molecular sizes of approximately 55 kDa and approximately 28 kDa were detected (FIG. 3).

### 2) Confirmation of Recognized Antigen

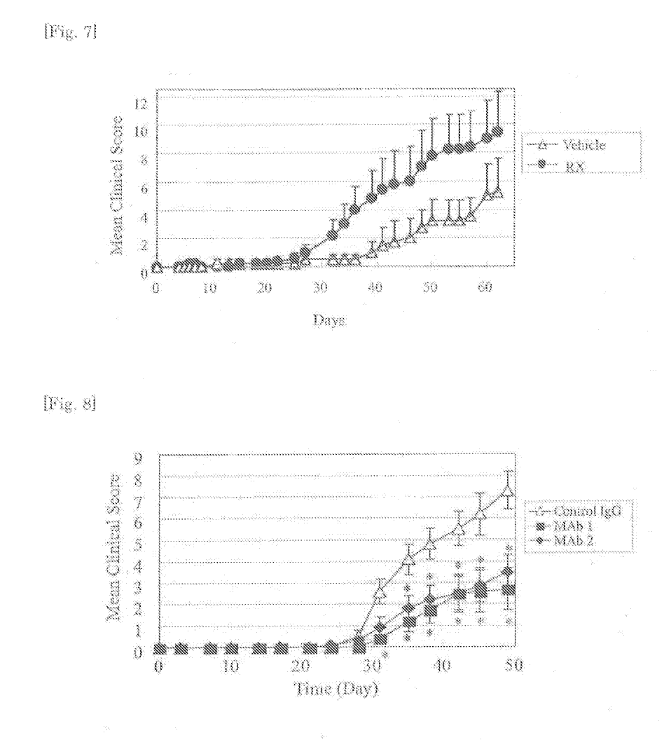

In order to confirm the molecular weight of the antigen recognized by MAb1, Western blot analysis was carried out. Specifically, the antigen purified by paragraph b) 1) of Example 2 was subjected to SDS-PAGE under non-reducing conditions and then transferred so a nitrocellulose membrane according to a routine method. After the transfer, the membrane was blocked with Block Ace (DS Pharma Biomedical Co., Ltd.). Biotin-labeled MAb1 was added thereto and reacted for 1 hour. The membrane was fully washed with PBS containing 0.1% Tween 20 and then reacted with HRP-labeled streptavidin. Subsequently, the membrane was fully washed with PBS containing 0.1% Tween 20. Then, a chemiluminescent substrate (SuperSignal West Dura Extended Duration Substrate; Thermo Fisher Scientific K.K.) was allowed to act thereon to detect the band of an MAb1-reactive protein. Results showed that MAb1 recognized the protein with a molecular size of approximately 55 kDa purified by affinity chromatography (FIG. 4). A sample treated in advance with glycosidase F (PNGase F proteomics Grade; Sigma-Aldrich Corp.) was separated by SDS-PAGE. After the electrophoresis, a band corresponding to the protein that was positive in Western blot analysis was excised from the silver-stained gel and digested with trypsin (Trypsin Gold; Promega K.K.) after reductive alkylation. The digestion product was mixed with α-cyano-4-hydroxycinnamic acid (α-CHCA) and subjected to MALDI-TOF-MS analysis using Oltraflex II™ (Bruker Daltonics K.K.), Protein analysis software (Mascot Server; Matrix Science Ltd.) was searched for the corresponding protein as to the molecular size of the obtained ionized fragment peak. Results showed that the band of the protein recognized by MAb1 was shown to correspond to the extramembranous region gp52SU of retrovirus MMTV envelope protein (MMTV env) (FIG. 5). Also, the protein with a molecular size of approximately 28 kDa was analyzed in the same way as above and consequently confirmed to be at least a portion of the extracellular region of MMTV env gp36TM.

### c) Preparation of Monoclonal Antibody Against RX Antigen

The purified RX protein was emulsified as an antigen in a complete Freund's adjuvant, and this emulsion was intraperitoneally administered to each BALB/c mouse. Booster immunization was performed intraperitoneally and through the tail vein to enhance the antibody titer. Three to 4 days after final immunization, the spleen was collected, and cells were isolated and fused with a mouse myeloma cell (P3-X63) line by the PEG method to obtain hybridomas. The hybridomas were screened for those producing antibodies having the ability to bind to the RX protein as follows; first, the purified RX protein was added to each well of a microplate and immobilized. Antibodies produced by the hybridomas were reacted with the immobilized RX protein. Subsequently, HRP-labeled secondary antibodies that recognized mouse IgG were reacted therewith. o-Phenylenediamine substrates were allowed to emit color. The reaction was terminated with 2 N sulfuric acid, and the absorbance was measured at a wavelength of 490 nm using a plate reader. As a result, monoclonal antibodies 2 and 3 (MAb2 and MAb3) binding to the purified RX protein were obtained. FIG. 2 shows results of confirming, by ELISA, the abilities of MAb2 and MAb3 purified using Protein G affinity columns and MAb1 obtained in paragraph a) of Example 2 to bind to the purified RX protein. In this ELISA method, TMB substrates (MP Biomedicals, LLC (Cappel)) were used as chromogenic substrates, and the absorbance was measured at a wavelength of 450 nm using a plate reader.

### Example 3

### Sequence Analysis of Monoclonal Antibody

In order to sequence the gene of MAb1, mRNA was extracted from the MAb1-producing hybridoma according to a routine method. Several types of 3′ end primers shown below were designed by selecting sequences identical to human and mouse antibody genes from the nucleic acid sequence (heavy chain constant region: Accession No. P20759; light chain: Accession No. L22653) of rat antibody gene known in the art. cDNA fragments were obtained by 5′-RACE RT-PCR (GeneRacer/SuperScript III, Invitrogen Corp.) with the above-mentioned mRNA as a template using primers for the 3′ end of a heavy chain CH3 nucleotide sequence (SEQ ID NO: 16 in the Sequence Listing (CH-R1): TCATTTACCCGGAGAGTGGGAGAGA) and for the 3′ end of a light chain CL nucleotide sequence (SEQ ID NO: 17 in the Sequence Listing (CLK-R1): CTAACACTCATTCCTGTTGAAGCTC). Each cDNA fragment was confirmed to have the fragment size of the corresponding region of the antibody gene by agarose gel electrophoresis analysis. Next, each cDNA fragment was inserted into a cloning vector (TOPO TA cloning, Invitrogen Corp.), and the full-length DNA was sequenced. The sequence of the 3′ region was separately confirmed by 3′-RACE. The nucleotide sequence of MAb1 heavy chain DNA is shown in SEQ ID NO: 18 (FIG. 15) in the Sequence Listing. The amino acid sequence of the antibody heavy chain is shown in SEQ ID NO: 19 (FIG. 16) in the Sequence Listing. The nucleotide sequence of MAb1 light chain DNA is shown in SEQ ID NO: 20 (FIG. 17) in the Sequence Listing. The amino acid sequence of the antibody light chain is shown in SEQ ID NO: 21 (FIG. 18) in the Sequence Listing. The amino acid sequences of MAb1 CDRs are shown in FIG. 23 (SEQ ID NOs: 22 to 27 in the Sequence Listing).

In order to express the genes of MAb1 heavy and light chains in cultured animal cells, the genes were inserted into a tandem expression vector pTandem-1 (Merck KGaA (Novagen)) containing an IRES sequence. Specifically, the light chain gene was first inserted into an upstream site flanked by NcoI and XhoI. Subsequently, the heavy chain gene was inserted into a site flanked by NheI and ClaI downstream of the IRES sequence to construct an MAb1 gene expression vector pFFF05. Freestyle™ CHO-S Cells (Invitrogen Corp.) were transduced with this expression vector. The secretion of the antibody protein into the culture supernatant was confirmed. MAb1 was purified from the culture supernatant using a Protein G column (GE Healthcare Bio-Sciences Corp.). This recombinant MAb1 was confirmed to cross-react with the RX protein, as in hybridoma-derived MAb1, by Western-blot analysis.

In the case of MAb2, degenerate primers (SEQ ID NO: 28 in the Sequence Listing: primer RF for (mouse) antibody sequence analysis: CCGCTAGCATCSARGTNMAGCTGSAGSAGTC; SEQ ID NO: 29 in the Sequence Listing: printer HR for (mouse) antibody sequence analyses: AGCGCTCTTGACCAGGCATCCTAGAGTCA; SEQ ID NO: 30 in the Sequence Listing: primer LF for (mouse) antibody sequence analysis: CCCCATGGAYATTGTGMTSACMCAPWCTMCA; SEQ ID NO: 31 in the Sequence Listing: primer LR for (mouse) antibody sequence analysis: CCCTCGAGTTCAACACTCATTCCTGTTGAAGCCTTGACG) Were prepared according to the method of Wang Z. et al. (Journal of Immunological Methods, 233, pp. 167-177, 2000). cDNA fragments reverse-transcribed from hybridoma-derived mRNA by RT-PCR using Superscript III (Invitrogen Corp.) were inserted into a cloning vector (TOPO TA cloning, Invitrogen Corp.) to sequence the DNAs of heavy and light chain variable regions. The nucleotide sequence of the heavy chain variable region DNA of MAb2 is shown in SEQ ID NO: 32 (FIG. 19) in the Sequence Listing. The ammo acid sequence of the heavy chain variable region of the antibody is shown in SEQ ID NO: 33 (FIG. 20) in the Sequence Listing. The nucleotide sequence of the light chain variable region DNA of MAb2 is shown in SEQ ID NO: 34 (FIG. 21) in the Sequence Listing, The amino acid sequence of the light chain variable region of the antibody is shown in SEQ ID NO: 35 (FIG. 22) in the Sequence Listing. The amino acid sequences of MAb2 CDRs are shown in FIG. 23 (SEQ ID Nos: 36 to 41 in the Sequence Listing).

The heavy and light chain variable region DNAs of MAb3 were sequenced in the same way as for MAb2. The nucleotide sequence of the heavy chain variable region DNA of MAb3 is shown in SEQ ID NO: 62 (FIG. 30) in the Sequence Listing. The amino acid sequence of the heavy chain variable region or the antibody is shown in SEQ ID NO: 63 (FIG. 31) in the Sequence Listing. The nucleotide sequence of the light chain variable region DNA of MAb3 is shown in SEQ ID NO: 61 (FIG. 32) in the Sequence Listing. The amino acid sequence of the light chain variable region of the antibody is shown in SEQ ID NO: 65 (FIG. 33) in the Sequence Listing. The amino acid sequences of MAb3 CDRs are shown in FIG. 33 (SEQ ID NOs: 66 to 71 in the Sequence Listing.

### Example 4

### Binding Affinity of Monoclonal Antibody for RX Protein

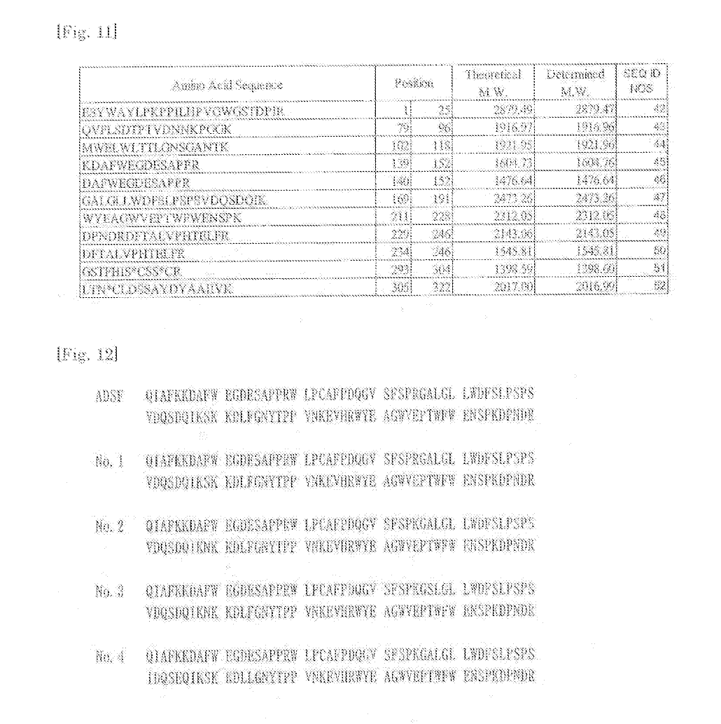

The binding affinity of each monoclonal antibody obtained in Example 2 for the RX protein was assayed using an SPR apparatus (ProteOn XPR36; Bio-Rad Laboratories, Inc.). MAb1 and MAb3 were each diluted to a concentration of 10 μg/ml with a 10 mM sodium acetate buffer solution (ph 4.5), while MAb2 was diluted to a concentration of 20 μg/ml using the same buffer solution. Each antibody was immobilized on a sensor chip (Sensor Chip GLM; Bio-Rad Laboratories, Inc.) by the amine coupling method. The chip was blocked by the addition of 1 M ethanolamine hydrochloride (pH 8.5). ProteOn PBS/Tween, pH 7.4 (PBS, pH 7.4, 0.005% Tween 20) was used as a running buffer solution. The purified RX protein was serially diluted and interacted as an analyte with the immobilized monoclonal antibody to determine the binding affinity. The association rate constant ka and the dissociation rate constant kd were calculated by kinetics analysis using ProteOn Manager™. Results showed that the obtained antibody had a binding affinity in the 10⁻¹⁰ M order for the RX protein (FIG. 6).

### Example 5

### Confirmation of Function of RX Protein on Exacerbation of Arthritis

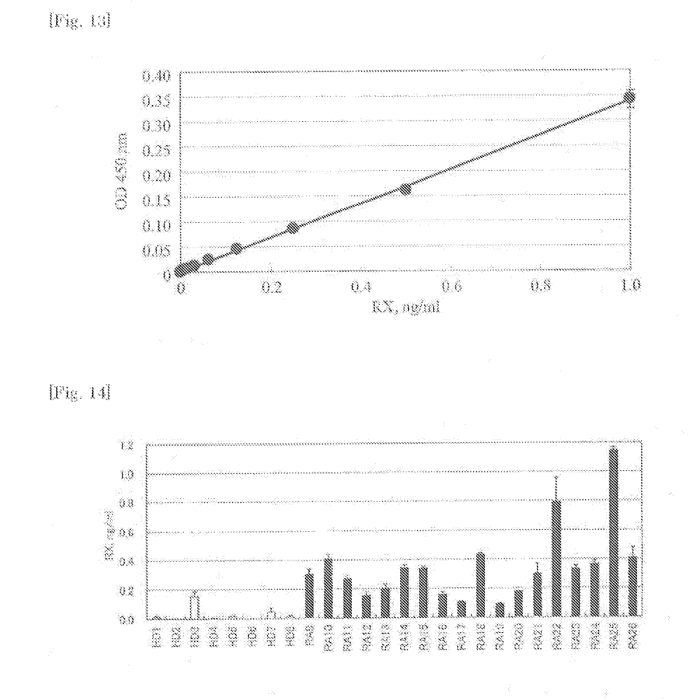

In order to confirm the involvement of the RX protein in arthritis, the experiment described below was conducted using a collagen-induced arthritis model. The collagen-induced arthritis was caused by intradermally administering an emulsion of bovine type II collagen and a complete Freund's adjuvant to the tail head of each male DBA/1 mouse and 2 weeks later, similarly administering thereto an emulsion of bovine type II collagen and an incomplete Freund's adjuvant (5 mice per group, RX and vehicle control groups). The RX protein purified from the culture supernatant of ADSF cells by the method described in paragraph b)1) of Example 2 was intravenously administered at a dose of 200 ng/mouse every three days from the day of the first sensitization with collagen. The exacerbation of arthritis was evaluated by scoring the degree of arthritis in each limb on a scale of 0 to 4 (5 stages) and evaluating the total score of limbs as the arthritis score of the individual. The results suggested that the administration of the RX protein exacerbated arthritis (FIG. 7). The error bar represents standard deviation (SE).

### Example 6

### Pharmaceutical Efficacy of Monoclonal Antibody on Arthritis Mouse Model

The monoclonal antibody MAb1 obtained in paragraph a) of Example 2 and the monoclonal antibody MAb1 obtained in paragraph c) of Example 2 were examined for their pharmacological function in a collagen-induced arthritis model

### a) Preparation of Collagen-Induced Arthritis Model

Collagen-induced arthritis was caused by intradermally administering an emulsion of bovine type II collagen and a complete Freund's adjuvant to the tail head of each male DBA/1 mouse and 3 weeks later, similarly administering thereto an emulsion of bovine type II collagen and an incomplete Freund's adjuvant. Each group included 10 mice. Each monoclonal antibody was intraperitoneally administered at a dose of 2 mg/kg every three days from the day of the second sensitization with collagen. IgG purified from normal rat serum was used as a negative control (control IgG antibody).

b) An Arthritis suppressive function (FIG. 8), and a) a bone destruction suppressive function (FIG. 9) were evaluated as indicators of pharmaceutical efficacy.

### b) Arthritis Suppressive Function

The exacerbation of arthritis was assessed by scoring the degree of arthritis in each limb as follows: 0=no sign of arthritis, 1=erythema and/or edema developed in one joint, 2=erythema and/or edema developed in two joints, 3=erythema and/or edema developed in the whole limb, and 4=joint deformity or rigidity. The total score of limbs were evaluated as the arthritis score of the individual. Results showed that the arthritis score of the control IgG antibody-administered group used as a negative control got worse over time, whereas remarkable suppression of arthritis was observed in the monoclonal antibody-administered group (FIG. 8). A significance test was conducted by the Wilcoxon's-test method relative to the control IgG antibody-administered group. MAb1 and MAb2 exhibited significant suppression of arthritis at days 31 and 35 or later, respectively (*: p<0.05). The error bar represents standard deviation (SE).

### c) Bone Destruction Suppressive Function

In the case of bone destruction, a collagen-induced arthritis model was prepared according to the method of paragraph a), and a rat's hindlimb was fixed in formalin. With reference to the soft X-ray photograph taken after the fixation, the degree of bone destruction in each of calcaneus, tarsal bone, and metatarsus was scored as follows: 0: normal, 1: mild, 2: moderate, and 3: severe (4 stages). The total score of limbs was evaluated as the bone destruction score of the individual. A significance test was conducted by the Wilcoxon's-test method relative to the control IgG antibody-administered group. Results showed that MAb1 was confirmed to have a significant bone destruction suppressive function (FIG. 9, #: P<0.05). The error bar represents standard deviation (SE).

### Example 7

### Expression of RX Protein and RX Gene in RX Patient

In order to examine the presence of the RX protein and the RX gene in RA patients, expression was confirmed by Western blot and MS analysis at the protein level and by Southern blot and gene sequence analysis at the gene level.

### a) Western Blot Analysis

The joint synovium excised from each RA patient during surgery was treated with collagenase and then cut finely. The pieces of the synovium were cultured for 2 to 5 days in an RPMI1640 medium containing 10% FCS. After the culture, the medium supernatant was collected and added to an MAb1-immobilized affinity column, followed by the elation of bound proteins. Then, the eluate was created using Proteoprep Immunoaffinity Albumin and IgG Depletion Kit (Sigma-Aldrich Corp., product No.: PROT-IA) to remove contaminating albumin and human IgG. An SDS sample buffer was added to the resulting affinity column eluate, and the mixture was heat-treated to prepare an electrophoresis sample. The electrophoresis was performed under non-reducing conditions according to the Laemmli method (Nature, 227: pp. 680-685 (1970)). After the electrophoresis, proteins were transferred from the gel to a nitrocellulose membrane (Bio-Rad Laboratories, Inc., Trans Blot; product No.: 162-0093) using a blotting apparatus (Bio-Rad Laboratories, Inc., SEMI DRY TRANSFER CELL; product No.: 170-3940. After the transfer, the membrane was blocked with Block Ace (DS Pharma Biomedical Co., Ltd.; product No.: UK-B80) and reacted with biotinylated MAb1 at a concentration of 1 μg/ml for 1 hour. The membrane was fully washed with PBS containing 0.1% Tween 20 and then reacted with HRP-labeled streptavidin (GE Healthcare Bio-Sciences Corp.: product No.: 1058765) for 1 hour. The membrane was further fully washed with PBS containing 0.1% tween 20 and then reacted using a chemiluminescent substrate Super Signal West Dura Extended Duration Substrate (Thermo Fisher Scientific K.K., product No.: 34075). Then, photographs were taken to detect an MAb1-reactive protein. FIG. 10 shows the results of Western blot on case Nos. 1 to 4 as a part of the analysis. Lane numbers RA1, RA2, RA3, and RA4 represent samples derived from RA patients RA1, RA2, RA3, and RA4, respectively. The RA patients exhibited a positive band of the same size as that of the ADSF cell-derived RX protein (lane S).

### b) Mass Spectral Analysis

The sample affinity-purified from the RA patient (RA1)-derived plasma described in paragraph a) of Example 7 was separated by SDS-PAGE under non-reducing conditions. A band corresponding to the protein that was MAb1-positive in Western blot analysis was excised from the gel and digested with trypsin after reductive alkylation. The digestion product was mixed with α-cyano-4-hydroxycinnamic acid (α-CHCA) and subjected to MALDI-TOF-MS analysis using Voyager-DE STR™ (Applied Biosystems, Inc.). A protein database was searched for a protein with a molecular size that corresponded to the obtained ionized fragment peak using a protein identification search engine MS-Fit (UCSF). As a result, the MAb1-reactive protein was confirmed to be the extramembranous region gp52SU of retrovirus MMTV envelope protein (MMTV env). FIG. 11 (SEQ ID NOs: 42 to 52 in the Sequence Listing) shows the amino acid sequences of peptide fragments that exhibited a match between the RA patient-derived protein and gp52SU.

### c) Southern Blot Analysis

ISOGEN reagent (Nippon Gene Co., Ltd.) was added to the joint synovium excised from each RA patient during surgery. The mixture was homogenized for 30 seconds using a homogenizer (IKA Japan K.K.). followed by RNA extraction. The total RNA samples thus obtained or the total RNA samples derived from the joint synovium of RA patients (purchased from Scottish Biomedical Ltd.) were used. These total RNA samples were each used as a template to synthesize cDNA using SuperScript VILO cDNA Synthesis Kit (Invitrogen Corp.), Genomic DMA was also purified from similarly separated aliquots of the tissue samples using Easy-DNA KIT (Invitrogen Corp.).

A gene sequence encoding MMTV env was detected as follows: PCR with the tissue sample-derived cDNA or genomic DNA mentioned above as a template was carried out under the following conditions using a set of primer 1 (SEQ ID NO: 53 in the Sequence Listing: 5′-CCAGATCGCCTTTAAGAAG-3′) and primer 2 (SEQ ID NO: 54 in the Sequence Listing: 5′-CTATCATTGGGATCCTTAGGAGAATT-3′) designed within the coding region of the MMTV env gene, and KOD FX DNA polymerase (Toyobo Co., Ltd.): thermal denaturation at 94° C. for 3 minutes, followed by 50 repetitive cycles each involving 94° C. for 30 seconds, 55° C. for 30 seconds, and 38° C. for 30 seconds. Each PCR amplification product was separated by agarose gel electrophoresis and then transferred to a nylon membrane. A positive band was detected by hybridization according to a routine method. Specifically, the amplification product was mildly shaken at 50° C. for 30 minutes in a prehybridization solution (DIG Easy Hyb; Roche Diagnostics K.K.). After replacement of the prehybridization solution, a thermally denatured probe for detection was added thereto, and hybridization was performed with mild shaking at 50° C. for 2 hours or longer. The probe for detection was prepared by DIG-labeling the 3′ end of an oligonucleotide represented by SEQ ID NO: 55 in the Sequence Listing (5′-TGCGCCTTCCCTGACCAAGGG-3′) using DIG Oligonucleotide 3′-End Labeling kit, 2nd Generation (Roche Diagnostics K.K.). After the hybridization, the membrane was washed at room temperature for 5 minutes twice with a 2×SSC (150 mM NaCl, 15 mM sodium citrate; pH 7.0) solution containing 0.1% SDS and subsequently washed at 50° C. for 15 minutes twice with a 0.5×SSC solution containing 0.1% SDS. Then, the membrane was blocked using DIG Wash and Block Buffer Set (Roche Diagnostics K.K.) and reacted with anti-digoxigenin-AP antibodies to detect a DNA fragment bound with the probe. As a result, positive bawds were detected from a plurality of RA patient-derived samples.

### d) Sequence Analysis of PCR-Positive Sample

The nucleotide sequence of DNA of each sample confirmed to be positive by the Southern blot analysis described in paragraph c) of Example 7 was analyzed using PRISM 3100-Avant Genetic Analyzer™ (Applied Biosystems, Inc.) by the direct sequencing of the PCR product or the subcloning of the PCR product into a vector. Results showed that all of the nucleotide sequences of the DNAs obtained from the samples derived from the RA patients RA5 to RA8) exhibited high homology to the nucleotide sequence of MMTV env. These nucleotide sequences were translated into amino acid sequencers. The resulting sequences were substantially identical to the amino acid sequence of the ADSF cell-derived RX protein, though the sequences had 0 to 4 amino acid mutations (FIG. 12: SEQ ID NOs: 56 to 59 in the Sequence Listing).

### Example 8

### Construction of Sandwich ELISA Assay on RX Protein

### a) Confirmation of Sandwich ELISA Assay on RX Protein

In order to provide a means of measuring the amount of RX protein in blood, a sandwich ELISA assay was established. Specifically, MAb1 was immobilized at a concentration of 2 μg/ml on a High Bind microplate for immunoassay (Corning Inc. (Costar), product No.: 3590). The microplate was blocked using Block Ace (DS Pharma Biomedical Co., Ltd.). After addition of each assay sample, the microplate was fully washed with PBS containing 0.1% Tween 30. Then, biotin-labeled MAb3 was added as an antibody for detection at a concentration of 1 μg/ml. Subsequently, the microplate was fully washed with PBS containing 0.13 Tween 20. Then, HRP-labeled streptavidin was allowed to act thereon. The microplate was further fully washed with PBS containing 0.1% Tween 20 and then reacted with a chromogenic substrate TMB. The chromogenic reaction was terminated with 0.1 N hydrochloric acid. Then, the light absorption was measured at a wavelength of 450 nm. A calibration curve prepared using the purified RX protein produced linearity in the range of 0.0625 ng/ml to 1 ng/ml, as shown in FIG. 13.

### b) Study Using Blood of RA Patient and Normal Subject

The amount of RX protein in blood was analyzed by the sandwich ERISA assay system mentioned above using the plasma samples of RA patients of 18 cases (RA9 to RA26) and 8 healthy human volunteers (HD1 to HD8). Results showed that the RX protein in blood was detected in the RA patients in an amount 11.5 times on average that in the normal subjects (FIG. 14), though expression was also seen in some normal subjects. A significance test based on the Student-T test also demonstrated that the amount of the RX protein in the blood of the RA patients was significantly larger than that in the normal subjects (p<0.001). This result showed that RA patients can be discriminated from normal subjects by measuring the amount of the RX protein in blood.

### Example 9

### Preparation and Functional Confirmation of Chimeric Antibody

Nucleotide sequences encoding the respective variable regions of the H chain (SEQ ID NO: 19) and L chain (SEQ ID NO: 21) of the rat antibody MAb1 sequenced in Example 3 were grafted into homologous sites in the nucleotide sequences encoding the H and L chain variable regions of human IgG1 to prepare the gene sequence of a rat-human chimeric antibody (hereinafter, referred to as “chimerized MAb1”). This antibody gene was incorporated into an expression vector for cultured animal cells according to the method of Example 3. HEK293 cells were transduced with the expression vector according to a routine method using a transfection reagent to transiently express chimerized MAb1. The antibody secreted into the culture supernatant at culture day 4 to 5 was added to a Protein A column. An adsorbed fraction was elated with a 100 mM glycine-HCl buffer solution (pH 2.8). The eluate was immediately neutralized using a 1 M tris-HCl buffer solution. The obtained chimerized MAb1 was analyzed for its binding affinity for the RX protein by SPR and consequently confirmed to exhibit binding affinity at the same level as that of the rat antibody MAb1 (FIG. 34).

### Example 10

### Preparation and Functional Confirmation of Humanized Antibody

### a) Design of Humanized Antibody Sequence

The H and L chains of human IgG1 having high homology to the amino acid sequences of the MAb1 H chain (SEQ ID NO: 19; FIG. 16) and L chain (SEQ ID NO: 21; FIG. 18) sequenced in Example 3 were selected. The H and L chain CDR sequences of MAb1 were grafted into homologous sites in the selected H and L chains of human IgG1 to design the sequence of MAb1 very similar to the human IgG1 antibody (hereinafter, referred to as “humanized MAb1”). In addition, on the basis of the molecular models of the predicted MAb1 variable regions, some amino acid residues in framework regions were substituted so as to stabilize the structures of the CDR sequences. Detailed procedures for the CDR grafting into homologous sites and the optimization of framework regions are as follows:

### I) Molecular Modeling of MAb1 Variable Region

The molecular modeling of the MAb1 variable regions was carried out according to a generally known homology modeling method (Methods in Enzymology, 203, 121-153, (1991). Specifically, the primary amino acid sequences of human immunoglobulin variable regions registered in Protean Data Bank (Nuc, Acid Res., 35, D301-D303 (2007)) were searched for sequences most homologous to the primary amino acid sequences of the MAb1 variable regions. PDB code: IZAN (chain L: hereinafter, referred to as a “IZAN light chain”) and POB ID: 2GHW (chain B; hereinafter, referred to as a “2GHW heavy chain”) exhibited the highest sequence homology to the light and heavy chain variable regions of MAb1, respectively, and were thus selected as models for structural prediction. The three-dimensional structures of framework regions were prepared (hereinafter, referred to as a “framework model”) by combining the coordinates of the IZAN light chain and 2GHW heavy chain corresponding to the MAb1 light and heavy chains, respectively. The CDR sequences of MAb1 were assigned as clusters 11A, 7A, 9A, 10A, and 10B to CDRL1, CDRL2, CDRL3, CDRH1, and CDRH2, respectively, according to the classification of Thornton et al. (J. Mol. Biol., 263, 800-815, (1996)). By contrast, kink type (8) was adopted for CDRH3 according to the H3 rule (FEBS letter, 399, 1-8 (1996). Next, the typical conformation of each CDR sequence was incorporated into the framework model to construct a three-dimensional structural model of the MAb1 variable regions (hereinafter, referred to as a “MAb1 structure model”). The MAb1 structure model thus obtained was subjected to molecular dynamics simulation using a protein three-dimensional structure prediction program Prime and a conformation search program MacroModel (Schroedinger, LLC) to determine the minimum kinetic energies of all atoms constituting the principal and side chains. In this way, the most stable structure or the MAb1 structure model was determined.

### 2) Design of Amino Acid Sequence of Humanized MAb1

CDR grafting for designing the primary amino acid sequence of the humanized MAb1 antibody was carried out according to the method of Queen C, et al. (Proc. Natl. Acad. Sci. USA, 86, 10029-10033 (1989)). Specifically, the amino acid sequences of the MAb1 framework regions were compared with the amino acid sequences of human antibody framework regions registered in the Kabat database (Nuc. Acid Res., 29, 205-206 (2001)). Results showed that an mAb58'CL antibody exhibited 77% sequence homology in the framework regions and was thus selected as an antibody providing human antibody framework regions (hereinafter, referred to as an “acceptor antibody”). The amino acid residues of framework regions in the acceptor antibody were compared with the amino acid sequences of the MAb1 framework regions to identify the positions of amino acid residues that did not match therebetween. The positions of these unmatched amino acid residues were projected onto the MAb1 structure model constructed in paragraph a)1) of Example 10 and thereby analyzed for their degree of intersection with the MAb1 CDR sequences according to the criteria of Queen et al. (Proc. Natl. Acad. Sci, USA, 86, 10029-10033 (1989)). Amino acid residues to be transferred from the MAb1 sequence to the acceptor antibody sequence (hereinafter, referred to as “donor residues”) were determined on the basis of unmatched amino acids in the framework regions presumed to be important for maintaining the three-dimensional structures of the CDR sequences. In order to secure diverse physicochemical properties, the amino acid sequence of humanized MAb1 was designed as ten H chain sequences (MAb1H1 to MAb1H10: SEQ ID NOs: 72 to 81; FIGS. 40 to 49) and five L chain sequences (MAb1L1 to MAb1L5: SEQ ID NOs: 82 to 86 in the Sequence Listing; FIGS. 50 to 54) by changing the transfer position of the donor residues.

### 3) Amino Acid Sequence of Humanized MAb1

The amino acid sequence of each humanized MAb1 thus designed is shown blow.

The H chain variable region of humanized MAb1 designed by replacing amino acid Nos. 19 (lysine), 23 (valine), 24 (glycine), 42 (lysine), 49 (alanine), 74 (serine), 75 (alanine), 77 (serine). 82 (glutamine), 88 (serine), 93 (threonine), 113 (valine), and 114 (methionine) counted from the N terminus of the H chain variable region of MAb1 represented by SEQ ID NO: 19 in the Sequence Listing with arginine, alanine, alanine, glycine, serine, asparagine, serine, asparagine, lysine, alanine, valine, threonine, and leucine, respectively, was designated as “MAb1H1” (amino acid sequence of SEQ ID NO: 91; SEQ ID NO: 72; FIG. 40). In the present invention, this region is also simply referred to as “H1”.

The H chain variable region of humanized MAb1 designed by replacing amino acid Nos. 19 (lysine), 23 (valine), 24 (glycine), 42 (lysine), 74 (serine), 75 (alanine), 77 (serine), 82 (glutamine), 88 (serine), 93 (threonine). 113 (valine), and 114 (methionine) counted from the N terminus of the H chain variable region of MAb1 represented by SEQ ID NO: 19 in the Sequence Listing with arginine, alanine, alanine, glycine, asparagine, serine, asparagine, lysine, alanine, valine, threonine, and leucine, respectively, was designated as “MAb1H2” (amino acid sequence of SEQ ID NO: 92; SEQ ID NO: 73; FIG. 41). In the present invention, this region is also simply referred to as “H2”.

The H chain variable region of humanized MAb1 designed by replacing amino acid Nos. 19 (lysine), 23 (valine), 42 (lysine), 35 (alanine), 77 (serine), 82 (glutamine), 88 (serine), 93 (threonine), 113 (valine), and 114 (methionine) counted from the N terminus of the H chain variable region of MAb1 represented by SEQ ID NO: 19 in the Sequence Listing with arginine, alanine, glycine, serine, asparagine, lysine, alanine, valine, threonine, and leucine, respectively, was designated as “MAb1H3” (amino acid sequence of SEQ ID NO: 93; SEQ ID NO: 74; FIG. 42). In the present invention, this region is also simply referred to as “H3”.

The H chain variable region of humanized MAb1 designed by replacing amino acid Nos. 19 (lysine), 23 (valine), 42 (lysine), 77 (serine), 82 (glutamine), 88 (serine), 93 (threonine), 113 (valine), and 114 (methionine) counted from the N terminus of the H chain variable region of MAb1 represented by SEQ ID NO: 19 in the Sequence Listing with arginine, alanine, glycine, asparagine, lysine, alanine, valine, threonine, and leucine, respectively, was designated as “MAb1H4” (amino acid sequence of SEQ ID NO: 94; SEQ ID NO: 75; FIG. 43). In the present invention, this region is also simply referred to as “H4”.

The H chain variable region of humanized MAb1 designed by replacing amino acid Nos. 19 (lysine), 23 (valine), 24 (glycine), 42 (lysine), 75 (alanine), 77 (serine), 82 (glutamine), 88 (serine), 93 (threonine), 113 (valine), and 114 (methionine) counted from the N terminus of the H chain variable region of MAb1 represented by SEQ ID NO: 19 in the Sequence Listing with arginine, alanine, alanine, glycine, serine, asparagine, lysine, alanine, valine, threonine, and leucine, respectively, was designated as “MAb1H5” (amine acid sequence of SEQ ID NO: 95; SEQ ID NO: 76; FIG. 44). In the present invention, this region is also simply referred to as “H5”.

The H chain variable region of humanized MAb1 designed by replacing amino acid Nos. 19 (lysine), 23 (valine), 42 (lysine), 71 (serine), 75 (alanine), 77 (serine), 82 (glutamine), 68 (serine), 93 (threonine), 113 (valine), and 114 (methionine) counted from the N terminus of the H chain variable region of MAb1 represented by SEQ ID NO: 19 in the Sequence Listing with arginine, alanine, glycine, asparagine, serine, asparagine, lysine, alanine, valine, threonine, and leucine, respectively, was designated as “MAb1H6” (amino acid sequence of SEQ ID NO: 96; SEQ ID NO: 77; FIG. 15). In the present invention, this region is also simply referred to as “H6”.

The H chain variable region of humanized MAb1 designed by replacing amino acid Nos. 19 (lysine), 23 (valine), 42 (lysine), 33 (alanine), 77 (serine), 88 (serine), 93 (threonine), 118 (saline), and 114 (methionine) counted from the 3 terminus of the H chain variable region of MAb1 represented by SEQ ID NO: 19 in the Sequence Listing with arginine, alanine, glycine, serine, asparagine, alanine, valine, threonine, and leucine, respectively, was designated as “MAb1H8” (ammo acid sequence of SEQ ID NO: 37; SEQ ID NO: 78; FIG. 46). In the present invention, this region is also simply referred to as “H8”.

The H chain variable region of humanized MAb1 designed by replacing amino acid Nos. 19 (lysine;, 33 (valine), 24 (glycine), 42 (lysine), 49 (alanine), 75 (alanine), 77 (serine), 82 (glutamine), 88 (serine), 93 (threonine), 113 (valine), and 114 (methionine) counted from the N terminus of the H chain variable region of MAb1 represented by SEQ ID NO: 19 in the Sequence Listing with arginine, alanine, alanine, glycine, serine, serine, asparagine, lysine, alanine, valine, threonine, and leucine, respectively, was designated as “MAb1H8” (amino acid sequence of SEQ ID NO: 98; SEQ ID NO: 79; FIG. 47). In the present invention, this region is also simply referred to as “H8”.

The H chain variable region of humanized MAb1 designed by replacing amino acid Nos. 13 (glutamine), 16 (arginine), 19 (lysine), 13 (valine), 24 (glycine), 42 (lysine), 75 (alanine), 77 (serine), 82 (glutamine), 86 (serine), 93 (threonine), 113 (valine), and 114 (methionine) counted from the N terminus of the H chain variable region of MAb1 represented by SEQ ID NO: 19 in the Sequence Listing with lysine, glycine, arginine, alanine, alanine, glycine, serine, asparaginic, lysine, alanine, saline, threonine, and leucine, respectively, was designated as “MAb1H9” (amino acid sequence of SEQ ID NO: 99; SEQ ID NO: 80: FIG. 48). In the present invention, this region is also simply referred to as “H9”.

The H chain variable region of humanized MAb1 designed by replacing amino acid Nos. 13 (glutamine), 16 (arginine), 19 (lysine), 23 (valine), 24 (glycine), 42 (lysine), 49 (alanine), 75 (alanine), 77 (serine), 82 (glutamine), 86 (serine), 93 (threonine), 113 (valine), and 114 (methionine) counted item the N terminus of the H chain variable region of MAb1 represented by SEQ ID NO: 19 in the Sequence Listing with lysine, glycine, arginine, alanine, alanine, glycine, serine, serine, asparagine, lysine, alanine, valine, threonine, and leucine, respectively, was designated as “MAb1H10” (amino acid sequence of SEQ ID NO: 100: SEQ ID NO: 81: FIG. 49). In the present invention, this region is also simply referred to as “H10”.

The L chain variable region of humanized MAb1 designed by replacing amino acid. Nos. 9 (alanine), 15 (leucine), 17 (glutamic acid), 18 (threonine), 22 (glutamic acid), 43 (serine), 43 (glutamine), 70 (glutamine), 72 (serine), 74 (lysine), 76 (asparagine), 77 (serine), 80 (serine), 83 (valine), 84 (serine), 85 (isoleucine), 87 (phenylalanine), 100 (alanine), 102 (alanine), 104 (leucine), 106 (leucine), and 10 (alanine) counted from the N terminus of the L chain variable region of MAb1 represented by SEQ ID NO: 21 in the Sequence Listing with serine, valine, aspartic acid, arginine, threonine, alanine, lysine, aspartic acid, threonine, threonine, serine, arginine, proline, phenylalanine, alanine, threonine, tyrosine, glutamine, threonine, valine, isoleucine, and threonine, respectively, was designated as “MAb1L1” (amino acid sequence of SEQ ID NO: 103; SEQ ID NO: 82; FIG. 50). In the present invention, this region is also simply referred to as “L1”.

The L chain variable region of humanized MAb1 designed by replacing amino acid Nos. 8 (alanine), 15 (leucine), 17 (glutamic acid), 18 (threonine), 22 (glutamic acid), 45 (glutamine), 70 (glutamine); 72 (serine), 74 (lysine), 76 (asparagine), 77 (serine), 80 (serine), 83 (valine), 84 (serine), 85 (isoleucine), 87 (phenylalanine), 100 (alanine), 102 (alanine), 104 (leucine), 106 (leucine), and 109 (alanine) counted from the N terminus of the L chain variable region of MAb1 represented by SEQ ID NO: 21 in the Sequence Listing with serine, valine, aspartic acid, arginine, threonine, lysine, aspartic acid, threonine, threonine, serine, arginine, proline, phenylalanine, alanine, threonine, tyrosine, glutamine, threonine, valine, isoleucine, and threonine, respectively, was designated as “MAb1L2” (amino acid sequence or SEQ ID NO: 104; SEQ ID NO: 83; FIG. 51). In the present invention, this region is also simply referred to as “L2”.

The L chain variable region of humanized MAb1 designed by replacing amino acid Nos. 9 (alanine), 15 (leucine), 17 (glutamic acid), 18 (threonine), 22 (glutamic acid), 70 (glutamine), 72 (serine), 74 (lysine), 76 (asparagine), 77 (serine), 80 (serine), 83 (valine), 84 (serine), 85 (isoleucine), 100 (alanine), 102 (alanine), 104 (leucine), 106 (leucine), and 109 (alanine) counted from the N terminus of the L chain variable region of MAb1 represented by SEQ ID NO: 21 in the Sequence Listing with serine, valine, aspartic acid, arginine, threonine, aspartic acid, threonine, threonine, serine, arginine, proline, phenylalanine, alanine, threonine, glutamine, threonine, valine, isoleucine, and threonine, respectively, was designated as “MAb1L3” (amino acid sequence of SEQ ID NO: 105; SEQ ID NO: 84; FIG. 52). In the present invention, this region is also simply referred to as “L3”.

The L chain variable region of humanized MAb1 designed by replacing amino acid Nos. 9 (alanine), 15 (leucine), 17 (glutamic acid), 18 (threonine), 22 (glutamic acid), 70 (glutamine), 72 (serine), 74 (lysine), 76 (asparagine), 77 (serine), 80 (serine), 83 (valine), 84 (serine), 85 (isoleucine), 100 (alanine), 102 (alanine), 106 (leucine), and 109 (alanine) counted from the N terminus of the L chain variable region of MAb1 represented by SEQ ID NO: 21 in the Sequence Listing with serine, valine, aspartic acid, arginine, threonine, aspartic acid, threonine, threonine, serine, arginine, proline, phenylalanine, alanine, threonine, glutamine, threonine, isoleucine, and threonine, respectively, was designated as “MAb1L4” (amino acid sequence of SEQ ID NO: 106; SEQ ID NO: 85; FIG. 53). In the present invention, this region is also simply referred to as “L4”.

The L chain variable region of humanized MAb1 designed by replacing amino acid Nos. 9 (alanine), 15 (leucine), 17 (glutamic acid), 18 (threonine), 22 (glutamic acid), 45 (glutamine), 70 (glutamine), 72 (serine), 74 (lysine), 76 (asparagine), 80 (serine), 83 (valine), 84 (serine), 85 (isoleucine), 67 (phenylalanine), 100 (alanine), 102 (alanine), 104 (leucine), 106 (leucine), and 109 (alanine) counted from the N terminus of the L chain variable region of MAb1 represented by SEQ ID NO: 21 in the Sequence Listing with serine, valine, aspartic acid, arginine, threonine, lysine, aspartic acid, threonine, threonine, serine, proline, phenylalanine, alanine, threonine, tyrosine, glutamine, threonine, valine, isoleucine, and threonine, respectively, was designated as “MAb1L5” (amino acid sequence of SEQ ID NO: 107; SEQ ID NO; 86; FIG. 54). In the present invention, this region is also simply referred to as “L5”.

### b) Construction of Vector for Humanized Antibody Heavy Chain Expression

Human IgG1 heavy chain constant region-encoding cDNA represented by the nucleotide sequence of SEQ ID NO: 87 (FIG. 55) in the Sequence Listing was chemically synthesized and used as a template in PCR using two primers represented by the nucleotide sequences of SEQ ID NOs: 59 and 90 (FIGS. 56 and 57), respectively, in the Sequence Listing to obtain a DNA fragment encoding a mouse IgM signal sequence and a human IgG1 heavy chain constant region (hereinafter, referred to as a “CH fragment”). The obtained CH fragment was inserted between the CMV promoter and the polyA addition signal sequence of the thymidine kinase gene in a vector for expression in animal cells to construct a vector pIgG1-CH containing an insert encoding the heavy chain protein constant region of the humanized antibody.

The heavy chain variable region-encoding sequences of the humanized MAb1 genes were prepared by chemically synthesizing or PCR-mutating cDNAs (SEQ ID NOs: 91 to 100 in the Sequence Listing: FIGS. 58 to 67) encoding the amino acid sequences of 10 candidate sequences designed in paragraph a)2) of Example 10. The obtained variable region-encoding cDNA fragments were each inserted into an Eco47III restriction site designed between the mouse IgM signal, sequence-encoding sequence and the human IgG1 heavy chain constant region-encoding sequence of the vector pIgG1-CH mentioned above to prepare vectors respectively expressing heavy chain proteins MAb1H1 to MAb1H10 serving as humanized MAb1 candidates. The obtained expression vectors were designated as “pMAb1-H1”, “pMAb1-H2”, “pMAb1-H3”, “pMAb1-H4”, “pMAb1-H5”, “pMAb1-H6”, “pMAb1-H7”, “pMAb1-H8”, “pMAb1-H9”, AND “pMAb1-H10”, respectively.

The procedures of preparing the expression vectors for the humanized MAb1 antibody heavy chain proteins are summarized in FIG. 37.

### c) Construction of Vector for Humanized Antibody Light Chain Expression

The light chain variable region DNA fragments of the humanized MAb1 genes were prepared by chemically synthesizing or PCR-mutating cDNAs (having the nucleotide sequences represented by SEQ ID NOs: 103 to 107 in the Sequence Listing; FIGS. 69 to 73) encoding the amino acid sequences of 5 candidate sequences designed in paragraph a)2) of Example 10. In the same way as in paragraph b) of Example 10, each light chain variable region DNA fragment thus obtained and a chemically synthesized human IgG1 light chain constant region-encoding cDNA fragment (hereinafter, referred to as a “CL fragment”) represented by the nucleotide sequence of SEQ ID NO: 101 (FIG. 68) in the Sequence Listing were inserted between the IgM signal sequence-encoding sequence and the polyA addition signal sequence of the thymidine kinase gene under the control of the CMV promoter to prepare vectors respectively expressing the light chain proteins MAb1L1 to MAb1L5 serving as humanized MAb1 candidates. The obtained expression vectors were designated as “pMAb1-L1”, “pMAb1-L2”, “pMAb1-L3”, “pMAb1-L4”, and “pMAb1-L5”, respectively.

### d) Preparation of Humanized Antibody

In order to confirm the functions of each humanized MAb1 consisting of heavy and light chains expressed from the humanized MAb1 heavy and light chain expression vectors prepared in paragraphs b) and c) of Example 10, one of the humanized MAb1 heavy chain protein expression vectors and one of the humanized MAb1 light chain protein expression vectors were mixed in each combination shown in FIG. 35. Exponentially growing HEK293 cells were transduced with the mixture by transfection and cultured at 37° C. for 4 to 5 days to transiently express the antibody proteins. Then, the culture supernatant was collected. The obtained culture supernatant was applied to Protein A affinity column chromatography. The column was washed with PBS, followed by the elution of an antibody-containing fraction with a 0.1 M glycine-HCl buffer solution (pH 2.8). The eluate was neutralized by the addition of a 1 M tris-HCl buffer solution in an amount 1/10 of the volume of the solution, and replaced with PBS or a histidine buffer solution by ultrafiltration.

The concentration of each humanized MAb1 sample thus purified was calculated on the basis of the molar absorption coefficient 13.8 of the human IgG antibody by measuring the absorbance at 280 nm using a spectrophotometer.

### e) Assay on Binding Affinity of Humanized Antibody for RX Protein

The binding affinity of each humanized MAb1 for the RX protein was assayed by SPR. Specifically, a Protein A-Protein G fusion protein Protein A/G (Thermo Fisher Scientific K.K.) was immobilized on the GLM sensor chip of ProteOn XPR36 (Bio-Rad Laboratories, Inc.) by the amine coupling method. Subsequently, each humanized MAb1 was added thereto and captured by the immobilized Protein A/G. Next, the RX protein was added thereto, and the association rate constant and the dissociation rate constant were determined using analysis software (ProteOn Manager version 3.0.1) from changes in sensorgram caused by association and dissociation. The dissociation constant, i.e., binding affinity, was calculated from the ratio therebetween. The binding affinity of each humanized MAb1 for the RX protein is shown in FIG. 36.

### Example 11

### Construction of ELISA Assay on RX Protein

In order to provide means of conveniently measuring the amount of the RX protein, ELISA assay was also studied by the direct adsorption method. The RX protein purified in paragraph b) of Example 3 was diluted 2-fold into a predetermined concentration and immobilized on a High Bind microplate for immunoassay (Corning Inc. (Costar), product No.: 3590). The microplate was blocked using Block Ace (DS Pharma Biomedical Co., Ltd.), The microplate was fully washed with PBS containing 0.1% Tween 20. Then, biotin-labeled MAb2 and MAb3 were added as antibodies for detection at a concentration of 1 μg/ml. Subsequently, the microplate was fully washed with PBS containing 0.1% Tween 20. Than, HRP-labeled streptavidin was allowed to act thereon. The microplate was further fully washed with PBS containing 0.1% Tween 20 and then reacted with a chromogenic substrate TMB. The chromogenic reaction was terminated with 0.1 N sulfuric acid. Then, the light absorption was measured at a wavelength of 450 nm. A calibration curve was prepared using the RX protein purified in paragraph, b) of Example 2. Results showed that use of MAb2 and MAb3 produced linearity in the ranges of 6.25 ng/ml to 400 ng/ml and 6.25 ng/ml to 200 ng/ml, respectively, as shown in FIGS. 38 and 39.

### Example 12

### Pharmaceutical Efficacy of Humanized Monoclonal Antibody of Arthritis Mouse Model

In order to confirm the pharmaceutical efficacy of five of the humanized MAb1 antibodies described in paragraph d) of Example 10 on an arthritis mouse model, each antibody was prepared by transient expression from 10 L of the culture solution. Each antibody's arthritis suppressive function was evaluated according to the method described in paragraphs a) and b) of Example 6. Results showed that the remarkable suppression of arthritis was observed in all of the humanized MAb1-administered groups compared with the control IgG antibody-administered group. FIG. 74 shows the arthritis score of each group at day 49 after sensitization. A significance test was conducted by the Wilcoxon's-test method relative to the control IgG-administered group (*; p<0.05; **: p<0.01). The error bar represents standard deviation (SE).

### Example 13

### Obtainment of Monoclonal Antibody MAb4 and Its Pharmaceutical Efficacy on Arthritis Mouse Model

MAb1 was obtained in the same way as in paragraph c) of Example 2 except that ADSF cells were used as antigens instead of the purified RX protein used in the preparation of MAb2 and MAb3. The heavy and light chain DNAs of the MAb1 antibody were sequenced according to the method of Example 3. The nucleotide sequence of the heavy chain variable region DMA of MAb1 is shown in SEQ ID NO: 108 (FIG. 75) in the Sequence Listing. The amino acid sequence of the heavy chain variable region of the antibody is shown in SEQ ID NO: 109 (FIG. 76) in the Sequence Listing. The nucleotide sequence of the light chain variable region DNA of MAb1 is shown in SEQ ID NO: 110 (FIG. 77) in the Sequence Listing. The amino acid sequence of the light chain variable region of the antibody is shown in SEQ ID NO: 111 (FIG. 78) in the Sequence Listing. The amino acid sequences of MAb1 CDRs are shown in FIG. 79 (SEQ ID Nos: 112 to 117).

The binding affinity of MAb1 for the RX protein was assayed by the method described in Example 4. Results showed that MAb4 had a binding affinity of 1.4×10⁻¹⁰ M for the RX protein.

MAb1 was examined for its arthritis suppressive function in a collagen-induced arthritis mouse model according to the method described in paragraphs a) and b) of Examples 6. Results showed that remarkable suppression of arthritis was observed in the MAb4-administered group (filled circle) compared with the control IgG-administered group (open triangle) (FIG. 80. A significance test was conducted by the Wilcoxon's-test method relative to the control IgG-administered group (*:p<0.05). The error bar represents standard deviation (SE).

ELISA assay based on the direct adsorption method of the RX protein using MAb4 was studied by the method described in Example 11. Results showed that linearity was produced in the range of 6.25 ng/ml to 200 ng/ml, shown in FIG. 81.

### Example 14

### Inhibitory Function of MAb1 on Cytokine Production in Inflamed Region

At the completion of the experiment of Example 12, limbs were excised from each mouse and frozen. Their tissues were disrupted using Shake Master NEO (BMS). For extraction, 5 μl of Cell lysis buffer (Bio-Rad Laboratories, Inc.) was added per mg of the disrupted tissues, and the extracts were centrifuged three times at 4° C. for 10 minutes. The obtained supernatant was used as a homogenate sample. Each homogenate sample was diluted 3-fold with PBS. The amounts of cytokines and chemokines produced were measured using Bio-plex Pro Cytokine Assay 10 plex (Bio-Rad Laboratories, Inc.). Results for IL-6 are shown in FIG. 82. Results for MCP-1 are shown in FIG. 83. Results showed that humanized MAb1 was confirmed to significantly inhibit the production of inflammatory cytokines or chemokines. A significance test was conducted by the Wilcoxon's-test method relative to the control IgG antibody-administered group (**: p<0.01, *: p<0.05 vs. control IgG). The significance test results for the control IgG antibody-administered group are shown relative to an untreated (normal) group (##: p<0.01 vs. normal). The error bar represents standard deviation (SE).

### INDUSTRIAL APPLICABILITY

Use of the antibody provided by the present invention achieves the treatment or prevention of autoimmune disease such as RA or arthritis and the examination or diagnosis of RA or the like.

### FREE TEXT OF SEQUENCE LISTING

SEQ ID NO: 1: Partial amino acid sequence 1 (FIG. 5) of ADSF cell-derived RX protein

SEQ ID NO: 2: Partial amino acid sequence 2 (FIG. 5) of ADSF cell-derived RX protein

SEQ ID NO: 3: Partial amino acid sequence 3 (FIG. 5) of ADSF cell-derived RX protein

SEQ ID NO: 4: Partial amino acid sequence 4 (FIG. 5) of ADSF cell-derived RX protein

SEQ ID NO: 5: Partial amino acid sequence 5 (FIG. 5) of ADSF cell-derived RX protein

SEQ ID NO: 6: Partial amino acid sequence 6 (FIG. 5) of ADSF cell-derived RX protein

SEQ ID NO: 7: Partial amino acid sequence 7 (FIG. 5) of ADSF cell-derived RX protein

SEQ ID NO: 8: Partial amino acid sequence 8 (FIG. 5) of ADSF cell-derived RX protein

SEQ ID NO: 9: Partial amino acid sequence 9 (FIG. 5) of ADSF cell-derived RX protein

SEQ ID NO: 10: Partial amino acid sequence 10 (FIG. 5) of ADSF cell-derived RX protein

SEQ ID NO: 11: Partial amino acid sequence 11 (FIG. 5) of ADSF cell-derived RX protein

SEQ ID NO: 12: Partial amino acid sequence 12 (FIG. 5) of ADSF cell-derived RX protein

SEQ ID NO: 13: Partial amino acid sequence 13 (FIG. 5) of ADSF cell-derived RX protein

SEQ ID NO 14: Nucleotide sequence (FIG. 24) of a gene encoding ADSF cell-derived RX protein gp73ED. A portion (the nucleotides Nos. 1 to 294 of SEQ ID NO: 60) corresponding to a signal sequence and 3′-TERMINAL 111 bases (except for the stop codon; the nucleotides Nos. 1954 to 2064 of SEQ ID NO: 60) were deleted from the nucleotide sequence of SEQ ID NO: 60.

SEQ ID NO: 15: Amino acid sequence (FIG. 25) of the ADSF cell-derived RX protein gp73ED. A signal sequence (amino acid Nos. 1 to 98 to SEQ ID NO: 61) and C-terminal 37 amino acids (amino acid Nos. 652 to 688 of SEQ ID NO: 610 were deleted from the amino acid sequence of SEQ ID NO: 61.

SEQ ID NO: 16: Primer CH-R1 for (rat) antibody sequence analysis

SEQ ID NO: 17: Primer CLK-R1 for (rat) antibody sequence analysis

SEQ ID NO: 18: Nucleotide sequence (FIG. 15) of cDNA encoding an MAb1 heavy chain

SEQ ID NO: 19: Amino acid sequence (FIG. 16) of the MAb1 heavy chain

SEQ ID NO: 20: Nucleotide sequence (FIG. 17) of cDNA encoding an MAb1 light chain

SEQ ID NO: 21: Amino acid sequence (FIG. 18) of the MAb1 light chain

SEQ ID NO: 22: Amino acid sequence (FIG. 23) of MAb1 heavy chain CDRH1

SEQ ID NO: 23: Amino acid sequence (FIG. 23) of MAb1 heavy chain CDRH2

SEQ ID NO: 24: Amino acid sequence (FIG. 23) of MAb1 heavy chain CDRH3

SEQ ID NO: 25: Amino acid sequence (FIG. 23) of MAb1 light chain CDRL1

SEQ ID NO: 26: Amino acid sequence (FIG. 23) of MAb1 light chain CDRL2

SEQ ID NO: 27: Amino acid sequence (FIG. 23) of MAb1 light chain CDRL3

SEQ ID NO: 28: Primer HF for (mouse) antibody sequence analysis

SEQ ID NO: 29: Primer HR for (mouse) antibody sequence analysis

SEQ ID NO; 30: Primer LF for (mouse) antibody sequence analysis

SEQ ID NO: 31: Primer LR for (mouse) antibody sequence analysis

SEQ ID NO: 32: Nucleotide sequence (FIG. 19) of cDNA encoding in MAb2 heavy chain variable region

SEQ ID NO; 33: Amino acid sequence (FIG. 20) of the MAb2 heavy chain variable region

SEQ ID NO: 34; Nucleotide sequence (FIG. 21) of cDNA encoding an MAb2 light chain variable region

SEQ ID NO: 35: Amino acid sequence (FIG. 22) of the MAb2 light chain variable region

SEQ ID NO: 36: Amino acid sequence (FIG. 23) of MAb2heavy chain CDRH1

SEQ ID NO: 37: Amino acid sequence (FIG. 23) of MAb2 heavy chain CDRH2

SEQ ID NO: 38: Amino acid sequence (FIG. 23) of MAb2 heavy chain CDRH3

SEQ ID NO; 39: Amino acid sequence (FIG. 23) of MAb2 light chain CDRL1

SEQ ID NO: 40: Amino acid sequence (FIG. 23) of MAb2 light chain CDRL2

SEQ ID NO: 41: Amino acid sequence (FIG. 23) of MAb2 light chain CDRL3

SEQ ID NO: 42: Partial amino acid sequence 1 (FIG, II) of RX protein derived from the plasma of an RA par lent:

SEQ ID NO: 43: Partial amino acid sequence 2 (FIG. 11) of RX protein derived from the plasma of an RA patient

SEQ ID NO: 44: Partial amino acid sequence 3 (FIG. 11) of RX protein derived from the plasma of an RA patient

SEQ ID NO: 45: Partial amino acid sequence 4 (FIG. 11) of RX protein derived from the plasma of an RA patient

SEQ ID NO: 46: Partial amino acid sequence 5 (FIG. 11) of RX protein derived from the plasma of an RA patient

SEQ ID NO: 47: Partial amino acid sequence 6 (FIG. 11) of RX protein derived from the plasma of an RA patient

SEQ ID NO: 48: Partial amino acid sequence 7 (FIG. 11) of RX protein derived from the plasma of an RA patient

SEQ ID NO: 49: Partial amino acid sequence 8 (FIG, 11) of RX protein derived from the plasma of an RA patient

SEQ ID NO; 50: Partial, amino acid sequence 9 (FIG. 11) of RX protein derived from the plasma of an RA patient

SEQ ID NO: 51: Partial amino acid sequence 10 (FIG. 11) of RX protein derived from the plasma of an RA patient

SEQ ID NO: 52: Partial amino acid sequence 11 (FIG. 11) of RX protein derived from the plasma of an RA patient

SEQ ID NO: 53: Primer 1 for PCR analysis of the RX protein gene

SEQ ID NO: 54: Printer 2 for PCR analysis of the RX protein gene

SEQ ID NO: 55: Probe for southern blot detection of the RX protein gene

SEQ ID NO: 56: Partial amino acid sequence 1 (No. 1 of FIG. 12) of RA patient-derived RX protein

SEQ ID NO: 57: Partial amino acid sequence 2 (No. 2 of FIG. 12) of RA patient-derived RX protein

SEQ ID NO: 58: Partial amino acid sequence 3 (No. 3 of FIG. 12) of RA par rent-derived RX protein SEQ ID NO: 59: Partial amino acid sequence 4 (No. 4 of FIG. 12) of RA patient-derived RX protein

SEQ ID NO: 60: Nucleotide sequence (FIG. 28) of a gene encoding the amino acid sequence of an ADSF cell-derived RX protein precursor containing a signal sequence and a C-terminal sequence

SEQ ID NO: 61: Amino acid sequence (FIG. 29) of the ADSF cell-derived RX protein precursor containing a signal sequence and a C-terminal sequence

SEQ ID NO: 62: Nucleotide sequence FIG. 30) of cDNA encoding an MAb3 heavy chain variable region

SEQ ID NO; 63: Amino acid sequence (FIG. 31) of the MAb3 heavy chain variable region

SEQ ID NO: 64: Nucleotide sequence (FIG. 32) of cDNA encoding an MAb3 light chain variable region

SEQ ID NO: 65: Amino acid sequence (FIG. 33) of the MAb3 light chain variable region

SEQ ID NO: 66: Amino acid sequence (FIG. 23) of MAb3 heavy chain CDRH1

SEQ ID NO: 67: Amino acid sequence (FIG. 23) of MAb3 heavy chain CDRH2

SEQ ID NO: 68: Amino acid sequence (FIG. 23) of MAb3 light chain CDRH3

SEQ ID NO: 69: Amino acid sequence (FIG. 23) of MAb3 light chain CDRL1

SEQ ID MO: 70: Amino acid sequence (FIG. 23) of MAb3 light chain CDRL2

SEQ ID NO: 71: Amino acid sequence (FIG. 23) of MAb3 light chain CDRL3

SEQ ID NO: 72: Amino acid sequence (FIG. 10) of the variable region of humanized MAb1 heavy chain H1

SEQ ID NO: 73: Amino acid sequence (FIG. 41) of the variable region of humanized MAb1 heavy chain H2

SEQ ID NO: 74: Amino acid sequence (FIG. 42) of the variable region of humanized MAb1 heavy chain H3

SEQ ID NO: 75: Amino acid sequence (FIG. 43) of the variable region of humanized MAb1 heavy chain H4

SEQ ID NO: 76: Amino acid sequence (FIG. 44) of the variable region of humanized MAb1 heavy chain H5

SEQ ID NO: 77: Amino acid sequence (FIG. 45) of the variable region of humanized MAb1 heavy chain H6

SEQ ID NO: 78: Amino acid sequence (FIG. 46) of the variable region of humanized MAb1 heavy chain H7

SEQ ID NO: 79: Amino acid sequence (FIG. 47) of the variable region of humanized MAb1 heavy chain H8

SEQ ID NO: 80: Amino acid sequence (FIG. 48) of the variable region of humanized MAb1 heavy chain H9

SEQ ID NO: 81: Amino acid sequence (FIG. 49) of the variable region of humanized MAb1 heavy chain H10

SEQ ID NO: 82: Amino acid sequence (FIG. 50) of the variable region of humanized MAb1 light chain L1

SEQ ID NO: 83: Amino acid sequence (FIG. 51) of the variable region of humanized MAb1 light chain L2

SEQ ID NO: 84: Amino acid sequence (FIG. 52) of the variable region of humanized MAb1 light chain L3

SEQ ID NO: 85: Ammo acid sequence (FIG. 53) of the variable region of humanized MAb1 light chain L4

SEQ ID NO: 86: Amino acid sequence (FIG. 54) of the variable region of humanized MAb1 light chain L5

SEQ ID NO: 87: Nucleotide sequence (FIG. 55) of cDNA encoding the amino acid sequence of the heavy chain constant region of human IgG1

SEQ ID NO: 88: Amino acid sequence of the heavy chain constant region of human IgG1

SEQ ID NO: 89: Nucleotide sequence (FIG. 56) of primer R for amplification of cDNA encoding the heavy chain constant region of human IgG1

SEQ ID NO: 90: Nucleotide sequence (FIG. 57) of primer R for amplification of cDNA encoding the heavy chain constant region of human IgG1

SEQ ID NO: 91: Nucleotide sequence (FIG. 58) of cDNA encoding the amino acid sequence of the variable region of humanized MAb1 heavy chain H1

SEQ ID NO: 92: Nucleotide sequence (FIG. 59) of cDNA encoding the amino acid sequence of the variable region of humanized MAb1 heavy chain H2

SEQ ID NO: 93: Nucleoside sequence (FIG. 60) of cDNA encoding the amino acid sequence of the variable region of humanized MAb1 heavy chain H3

SEQ ID NO: 94: Nucleotide sequence (FIG. 61) of cDNA encoding the amino acid sequence of the variable region of humanized MAb1 heavy chain H4

SEQ ID NO: 95: Nucleotide sequence (FIG. 62) of cDNA encoding the amino acid sequence of the variable region of humanized MAb1 heavy chain H5

SEQ ID NO: 96: Nucleotide sequence (FIG. 63) of cDNA encoding the amino acid sequence of the variable region of humanized MAb1 heavy chain H6

SEQ ID NO: 97: Nucleotide sequence (FIG. 64) of cDNA encoding the amino acid sequence of the variable region of humanized MAb1 heavy chain H7

SEQ ID NO: 98: Nucleotide sequence (FIG. 65) of cDNA encoding the amino acid sequence of the variable region of humanized MAb1 heavy chain H8

SEQ ID NO: 99: Nucleotide sequence (FIG. 66) of cDNA encoding the amino acid sequence of the variable region of humanized MAb1 heavy chain H9

SEQ ID NO: 100: Nucleotide sequence (FIG. 67) of

cDNA encoding the amino acid sequence of the variable region of humanized MAb1 heavy chain H10

SEQ ID NO: 101: Nucleotide sequence (FIG. 68) of cDNA encoding the amino acid sequence of the light chain constant region of human IgG1

SEQ ID NO: 102: Amino acid sequence of the light chain constant region of human IgG1

SEQ ID NO: 103: Nucleotide sequence (FIG. 69) of cDNA encoding the amino acid sequence of the variable region of humanized MAb1 light chain L1.

SEQ ID NO: 104: Nucleotide sequence (FIG. 70) of cDNA encoding the amino acid sequence of the variable region of humanized MAb1 light chain L2

SEQ ID NO: 105: Nucleotide sequence (FIG. 71) of cDNA encoding the amino acid sequence of the variable region of humanized MAb1 light chain L3

SEQ ID NO: 106: Nucleotide sequence (FIG. 72) of cDNA encoding the amino acid sequence of the variable region of humanized MAb1 light chain L4

SEQ ID NO: 107: Nucleotide sequence (FIG. 73) of cDNA encoding the amino acid sequence of the variable region of humanized MAb1 light chain L5

SEQ ID NO: 108: Nucleotide sequence (FIG. 75) of cDNA encoding the amino acid sequence of an MAb4 heavy chain variable region

SEQ ID NO: 109: Amino acid sequence (FIG. 76) of the MAb1 heavy chain variable region

SEQ ID NO: 110: Nucleotide sequence (FIG. 77) of cDNA encoding the amino acid sequence of an MAb1 light chain variable region

SEQ ID NO: 111: Amino acid sequence (FIG. 78) of the MAb1 light chain variable region

SEQ ID NO: 112: Amino acid sequence (FIG. 79) of MAb1 heavy chain CDRH1

SEQ ID HO: 113: Amino acid sequence (FIG. 79) of MAb1 heavy chain CDRH2

SEQ ID NO: 114: Amino acid sequence (FIG. 79) of MAb4 heavy chain CDRH3

SEQ ID NO: 115: Amino acid sequence (FIG. 79) of MAb4 heavy chain CDRL1

SEQ ID NO: 116: Amino acid sequence (FIG. 79) of MAb4 heavy chain CDRL2

SEQ ID NO: 117: Amino acid sequence (FIG. 79) of MAb4 heavy chain CDRL3

1. An antibody that recognizes a polypeptide comprising any one of the following amino acid sequences (I) to (III) and has an anti-arthritic function, or a functional fragment thereof:
(I) the amino acid sequence represented by SEQ ID NO: 15 in the Sequence Listing:
(II) the amino acid sequence that is encoded by the nucleotide sequence of a nucleic acid hybridizing under stringent conditions to a nucleic acid having a nucleotide sequence complementary to a nucleotide sequence encoding the amino acid sequence represented by SEQ ID NO: 15 in the Sequence Listing, and is of a polypeptide that causes the onset and/or exacerbation of arthritis; and
(III) the amino acid sequence that comprises an amino acid sequence represented by SEQ ID NO: 15 in the Sequence Listing having the substitution, deletion, addition, or insertion of one to several amino acids, and is of a polypeptide that causes the onset and/or exacerbation of arthritis.

2. The antibody or the functional fragment thereof according to claim 1, wherein the polypeptide has a molecular weight of (I) 50 to 55 k, (II) 50 to 55 k and 25 to 30 k, or (III) 70 to 75 k under non-reducing conditions of SDS-PAGE.

3. The antibody or the functional fragment thereof according to claim 1, wherein the antibody or the functional fragment thereof suppresses bone destruction.

4. The antibody or the functional fragment thereof according to claim 3, wherein bone destruction is a process in a collagen-induced arthritis non-human animal model.

5. The antibody or the functional fragment thereof according to claim 1, wherein the anti-arthritic function works in a collagen-induced arthritis non-human animal model.

6. The antibody or the functional fragment thereof according to claim 1, wherein the polypeptide exacerbates arthritis in a collagen-induced arthritis non-human animal model.

7. The antibody or the functional fragment thereof according to claim 1, wherein the polypeptide is capable of being detected in a collagen-induced arthritis mouse model.

8. The antibody or the functional fragment thereof according to claim 1, wherein the antibody or the functional fragment thereof inhibits cytokine production in an inflamed (body) region.

9. The antibody or the functional fragment thereof according to claim 8, wherein the cytokine is an inflammatory cytokine and/or a chemokine.

10. The antibody or the functional fragment thereof according to claim 8, wherein the inflamed (body) region is an affected part in a collagen-induced arthritis non-human animal model.

11. The antibody or the functional fragment thereof according to claim 4, wherein the non-human animal is a mouse.

12. The antibody or the functional fragment thereof according to claim 1, wherein the antibody or the functional fragment thereof recognizes the polypeptide comprising the amino acid sequence represented by SEQ ID NO: 15 in the Sequence Listing.

13. The antibody or the functional fragment thereof according to claim 1, wherein the antibody consists of a heavy chain comprising CDRH1 consisting of the amino acid sequence represented by SEQ ID NO: 22 in the Sequence Listing, CDRH2 consisting of the amino acid sequence represented by SEQ ID NO: 23 in the Sequence Listing, and CDRH3 consisting of the amino acid sequence represented by SEQ ID NO: 24 in the Sequence Listing, and a light chain comprising CDRL1consisting of the amino acid sequence represented by SEQ ID NO: 25 in the Sequence Listing, CDRL2 consisting of the amino acid sequence represented by SEQ ID NO: 26 in the Sequence Listing, and CDRL3 consisting of the amino acid sequence represented by SEQ ID NO: 27 in the Sequence Listing.

14. The antibody or the functional fragment thereof according to claim 1, wherein the antibody consists of a heavy chain comprising CDRH1 consisting of the amino acid sequence represented by SEQ ID NO: 36 in the Sequence Listing, CDRH2 consisting of the amino acid sequence represented by SEQ ID NO: 37 in the Sequence Listing, and CDRH3 consisting of the amino acid sequence represented by SEQ ID NO: 38 in the Sequence Listing, and a light chain comprising CDRL1consisting of the amino acid sequence represented by SEQ ID NO: 39 in the Sequence Listing, CDRL-2 consisting of the amino acid sequence represented by SEQ ID NO: 40 in the Sequence Listing, and CDRL3 consisting of the amino acid sequence represented by SEQ ID NO: 41 in the Sequence Listing.

15. The antibody or the functional fragment thereof according to claim 1, wherein the antibody consists of a heavy chain comprising CDRH1 consisting of the amino acid sequence represented by SEQ ID NO: 66 in the Sequence Listing, CDRH2 consisting of the amino acid sequence represented by SEQ ID NO: 67 in the Sequence Listing, and CDRH3 consisting of the amino acid sequence represented by SEQ ID NO: 68 in the Sequence Listing, and a light chain comprising CDRL1 consisting of the amino acid sequence represented by SEQ ID NO: 69 in the Sequence Listing, CDRL2 consisting of the amino acid sequence represented by SEQ ID NO: 70 in the Sequence Listing, and CDRL3 consisting of the amino acid sequence represented by SEQ ID NO: 71 in the Sequence Listing.

16. The antibody or the functional fragment thereof according to claim 1, wherein the antibody consists of a heavy chain comprising CDRH1 consisting of the amino acid sequence represented by SEQ ID NO: 112 in the Sequence Listing, CDRH2 consisting of the amino acid sequence represented by SEQ ID NO: 113 in the Sequence Listing, and CDRH3 consisting of the amino acid sequence represented by SEQ ID NO: 114 in the Sequence Listing, and a light chain comprising CDRL1 consisting of the amino acid sequence represented by SEQ ID NO: 115 in the Sequence Listing, CDRL2 consisting of the amino acid sequence represented by SEQ ID NO: 116 in the Sequence Listing, and CDRL3 consisting of the amino acid sequence represented by SEQ ID NO: 117 in the Sequence Listing.

17. An antibody or the functional fragment thereof wherein the antibody comprises heavy and light chains comprising amino acid sequences 95% or higher identical to the amino acid sequences of the heavy and light chains, respectively, of an antibody according to claim 13 and recognizes the polypeptide.

18. An antibody or the functional fragment thereof wherein the antibody or the functional fragment thereof binds to a site on an antigen recognized by an antibody or a functional fragment thereof according to claim 13.

19. An antibody or the functional fragment thereof wherein the antibody or the functional fragment thereof competes with an antibody or a functional fragment thereof according to claim 13 for binding to the polypeptide.

20. The antibody or the functional fragment thereof of claim 1 wherein the antibody is a chimeric antibody.

21. The antibody or the functional fragment thereof of claim 1 wherein the antibody is a humanized antibody.

22. The antibody or the functional fragment thereof of claim 1 wherein the antibody is a human antibody.

23. Any one of the following nucleic acids (I) to (III):
(I) a nucleic acid comprising a nucleotide sequence encoding a partial or whole amino acid sequence of the heavy or fight chain of an antibody according to claim 1;
(II) a nucleic acid consisting of a nucleotide sequence comprising a nucleotide sequence encoding a partial or whole amino acid sequence of the heavy or light chain of an antibody according to claim 1; and
(III) a nucleic acid consisting of a nucleotide sequence encoding a partial or whole amino acid sequence of the heavy or light chain of an antibody according to claim 1.

24. A recombinant vector containing an insert of a nucleic acid according to claim 23.

25. A recombinant cell containing a nucleic acid according to claim 23 introduced therein.

26. A cell producing an antibody according to claim 1.

27. A method for producing an antibody or a functional fragment thereof, comprising the following steps (I) and (II):
(I) culturing a cell according to claim 25; and
(II) collecting the antibody or the functional fragment thereof from the cultures obtained in step (I).

28. An antibody or the functional fragment thereof obtained by the method according to claim 27.

29. A modified form of an antibody or a functional fragment thereof according to claim 1.

30. A pharmaceutical composition comprising an antibody or a functional fragment thereof according to claim 1 as an active ingredient.

31. The pharmaceutical composition according to claim 30, wherein the pharmaceutical composition is a therapeutic or prophylactic drug for autoimmune disease in an individual expressing the polypeptide.

32. The pharmaceutical composition according to claim 31, wherein the autoimmune disease is rheumatoid arthritis.

33. The pharmaceutical composition according to claim 30, wherein the pharmaceutical composition is a therapeutic or prophylactic drag for arthritis an individual expressing the polypeptide.

34. A composition for examination or diagnosis of rheumatoid arthritis, comprising an antibody or a functional fragment thereof according to claim 1.

35. The antibody or the functional fragment thereof according to claim 13, wherein the heavy chain variable region comprises a peptide represented by an amino acid sequence described in any one of SEQ ID NOs: 72 to 81 (FIGS. 40 to 49) in the Sequence Listing, and the light chain variable region comprises a peptide represented by an amino acid sequence described in any one of SEQ ID NOs: 82 to 86 (FIGS. 50 to 54) in the Sequence Listing.

36. The antibody or the functional fragment thereof according to claim 13, wherein the heavy chain variable region is a peptide represented by an amino acid sequence described in any one of SEQ ID NOs: 72 to 81 (FIGS. 40 to 49) in the Sequence Listing, and the light chain variable region is a peptide represented by an amino acid sequence described in any one of SEQ ID NOs: 82 to 86 (FIGS. 50 to 54) in the Sequence Listing.

37. The antibody or the functional fragment thereof according to claim 13, wherein the heavy chain variable region is represented by an amino acid sequence consisting of amino acid Nos. 20 to 138 of SEQ ID NO: 19 (FIG. 16) in the Sequence Listing, and the light chain variable region is represented by an amino acid sequence consisting of amino acid Nos. 21 to 128 of SEQ ID NO: 21 (FIG. 18) in the Sequence Listing.

38. The antibody or the functional fragment thereof according to claim 35, wherein the heavy chain variable region comprises a peptide represented by an amino acid sequence selected from the group consisting of the amino acid sequences described in SEQ ID NOs: 72 to 74, 76, and 79 to 81 (FIGS. 40 to 42, 44, and 47 to 49) in the Sequence Listing, and the light chain variable region comprises a peptide represented by the amino acid sequence of SEQ ID NO: 82 (FIG. 50) in the Sequence Listing.

39. The antibody or the functional fragment thereof according to claim 36, wherein the heavy chain variable region is a peptide represented by an amino acid sequence selected from the group consisting of the amino acid sequences described in SEQ ID NOs: 72 to 74, 76, and 79 to 81 (FIGS. 40 to 42, 44, and 47 to 49) in the Sequence Listing, and the light chain variable region is a peptide represented by the amino acid sequence of SEQ ID NO: 82 (FIG. 50) in the Sequence Listing.

40. The antibody or the functional fragment thereof according to claim 35, wherein the heavy chain variable region comprises a peptide represented by an amino acid sequence selected from the group consisting of the amino acid sequences described in SEQ ID NOs: 72 to 74 and 76 to 78 (FIGS. 40 to 42 and 44 to 46) in the Sequence Listing, and the light chain variable region comprises a peptide represented by the amino acid sequence of SEQ ID NO: 83 (FIG. 51) in the Sequence Listing.

41. The antibody or the functional fragment thereof according to claim 36, wherein the heavy chain variable region is a peptide represented by an amino acid sequence selected from the group consisting of the amino acid sequences described in SEQ ID NOs: 72 to 74, and 76 to 78 (FIGS. 40 to 42 and 44 to 46) in the Sequence Listing, and the light chain variable region is a peptide represented by the amino acid sequence of SEQ ID NO: 83 (FIG. 51) in the Sequence Listing.

42. The antibody or the functional fragment thereof according to claim 35, wherein the heavy chain variable region comprises a peptide represented by an amino acid sequence described in any one of SEQ ID NOs: 72 to 74 (FIGS. 40 to 42) in the Sequence Listing, and the light chain variable region comprises a peptide represented by an amino acid sequence described in SEQ ID NO: 84 (FIG. 52) in the Sequence Listing.

43. The antibody or the functional fragment thereof according to claim 36, wherein the heavy chain variable region is a peptide represented by an amino acid sequence described in any one of SEQ ID NOs: 72 to 74 (FIGS. 40 to 42) in the Sequence Listing, and the light chain variable region is a peptide represented by an amino acid sequence described in SEQ ID NO: 84 (FIG. 52) in the Sequence Listing.

44. The antibody or the functional fragment thereof according to claim 35, wherein the heavy chain variable region comprises a peptide represented by the amino acid sequence of SEQ ID NO: 75 (FIG. 43) in the Sequence Listing, and the light chain variable region comprises a peptide represented by the amino acid sequence of SEQ ID NO: 85 (FIG. 53) in the Sequence Listing.

45. The antibody or the functional fragment thereof according to claim 36, wherein the heavy chain variable region is a peptide represented by the amino acid sequence of SEQ ID NO: 75 (FIG. 43) in the Sequence Listing, and the light chain variable region is a peptide represented by the amino acid sequence of SEQ ID NO: 85 (FIG. 53) in the Sequence Listing.

46. The antibody or the functional fragment thereof according to claim 35, wherein the heavy chain variable region comprises a peptide represented by an amino acid sequence selected from the group consisting of the amino acid sequences described in SEQ ID NOs: 73, 74, 76, and 77 (FIGS. 41, 42, 44 and 45) in the Sequence Listing, and the light chain variable region comprises a peptide represented by the amino acid sequence of SEQ ID NO: 86 (FIG. 54) in the Sequence Listing.

47. The antibody or the functional fragment thereof according to claim 36, wherein the heavy chain variable region is a peptide represented by an amino acid sequence selected from the group consisting of the amino acid sequences described in SEQ ID NOs: 73, 74, 76 and 77 (FIGS. 41, 42, 44 and 45) in the Sequence Listing, and the light chain variable region is a peptide represented by the amino acid sequence of SEQ ID NO: 86 (FIG. 54) in the Sequence Listing.

48. An antibody selected from the following (i) to (xxi), or a functional fragment thereof:
(i) an antibody (T13) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region:
(ii) an antibody (T14) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;
(iii) an antibody (T15) that consists of a heavy chain having a variable region consisting of the amino acid sequence represented by SEQ ID NO: 76 (FIG. 44) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region;
(iv) an antibody (T8) that consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;
(v) an antibody (T9) that consists of a heavy chain having a variable region consisting of die amino acid sequence represented by SEQ ID NO: 74 (FIG. 42) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region:
(vi) an antibody (T10) that consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region;
(vii) an antibody (T11) that consists of a heavy chain having a variable region consisting of the amino acid sequence represented by SEQ ID NO: 74 (FIG. 42) in the Sequence Listing and a human IgG1-derived constant region, and a light chain having a variable region consisting of the amino acid sequence represented by SEQ ID NO: 86 (FIG. 54) in the Sequence fisting and a human IgG1-derived constant region;
(viii) an antibody (T18) that consists of a heavy chain having a variable region consisting of the amino acid sequence represented by SEQ ID NO: 78 (FIG. 46) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;
(ix) an antibody (T12) that consists of a heavy chain having a variable region consisting of the amino acid sequence represented by SEQ ID NO: 75 (FIG. 43) in the Sequence Listing and a human IgG1-derived constant region, and a light chain having a variable region consisting of the amino acid sequence represented by SEQ ID NO: 85 (FIG. 53) in the Sequence Listing and a human IgG1-derived constant region;
(x) air antibody (T1) that consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;
(xi) an antibody (T2) that consists of a heavy chain having a variable region consisting of the amino acid sequence represented by SEQ ID NO: 72 (FIG. 40) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;
(xii) an antibody (T3) that consists of a heavy chain having a variable region consisting of the amino acid sequence represented by SEQ ID NO: 72 (FIG. 40) in the Sequence lasting and a human IgG1-derived constant region, and a light chain having a variable region consisting of the amino acid sequence represented by SEQ ID NO: 84 (FIG. 52) in the Sequence Listing and a human IgG1-derived constant region;
(xiii) an antibody (T4) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;
(xiv) an antibody (T5) that consists of a heavy chain having a variable region consisting of the amino acid sequence represented by SEQ ID NO: 73 (FIG. 41) in the Sequence Listing and a human IgG1-derived constant region, and a light chain having a variable region consisting of die amino acid sequence represented by SEQ ID NO: 83 (FIG. 51) in the Sequence Listing and a human IgG1-derived constant region;
(xv) an antibody (T6) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 84 (FIG. 52) in the Sequence Listing and a human IgG1-derived constant region:
(xvi) an antibody (T7) that consists of a heavy chain having a variable region consisting of the amino acid sequence represented by SEQ ID NO: 73 (FIG. 41) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region;
(xvii) an antibody (T6) that consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 83 (FIG. 51) in the Sequence Listing and a human IgG1-derived constant region;
(xviii) an antibody (T17) that consists of a heavy chain having a variable region consisting of the amino acid sequence represented by SEQ ID NO: 77 (FIG. 45) in the Sequence Listing and a human IgG1-derived constant region, and a light chain having a variable region consisting of the amino acid sequence represented by SEQ ID NO: 86 (FIG. 54) in the Sequence Listing and a human IgG1-derived constant region;
(xix) an antibody (T19) that consists of a heavy chain having a variable region consisting of the amino acid sequence represented by SEQ ID NO: 79 (FIG. 47) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region;
(xx) an antibody (T20) that consists of a heavy chain having a variable region consisting of the amino acid sequence represented by SEQ ID NO: 80 (FIG. 48) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region; and
(xxi) an antibody (T21) that consists of a heavy chain having a variable region consisting of the amino acid sequence represented by SEQ ID NO: 81 (FIG. 49) in the Sequence Listing and a human IgG1-derived constant region, and a light chain having a variable region consisting of the amino acid sequence represented by SEQ ID NO: 82 (FIG. 50) in the Sequence Listing and a human IgG1-derived constant region.

49. An antibody or the functional fragment thereof wherein the antibody comprises heavy and light chains comprising amino acid sequences 95% or higher identical to the amino acid sequences of the heavy and light chains, respectively, of an antibody according to claim 35.

50. An antibody or the functional fragment thereof wherein the antibody or the functional fragment thereof binds to a site on an antigen recognized by an antibody or a functional fragment thereof according to claim 35.

51. An antibody or the functional fragment thereof wherein the antibody or the functional fragment thereof competes with an antibody or a functional fragment thereof according to claim 35.

52. Any one of the following nucleic acids (I) to (III):
(I) a nucleic acid comprising a nucleotide sequence encoding a partial or whole amino acid sequence of the heavy or light chain of an antibody according to claim 35;
(II) a nucleic acid consisting of a nucleotide sequence comprising a nucleotide sequence encoding a partial or whole amino acid sequence of the heavy or light chain of an antibody according to claim 35; and
(III) a nucleic acid consisting of a nucleotide sequence encoding a partial or whole amino acid sequence of the heavy or light chain of an antibody according to claim 35.

53. The nucleic acid according to claim 52, wherein the nucleotide sequence encoding a partial or whole amino acid sequence of the heavy chain of an antibody is a nucleotide sequence represented by any one of SEQ ID NOs: 91 to 100 (FIGS. 58 to 67) in the Sequence Listing, and the nucleotide sequence encoding a partial or whole amino acid sequence of the light chain of an antibody is a nucleotide sequence represented by any one of SEQ ID NOs: 103 to 107 (FIGS. 69 to 73) in the Sequence Listing.

54. A recombinant vector containing an insert of a nucleic acid according to claim 52.

55. A recombinant cell containing a nucleic acid according to claim 52 introduced therein.

56. A cell producing an antibody according to claim 35.

57. A method for producing an antibody or a functional fragment thereof comprising the following steps (I) and (II):
(I) culturing a cell according to claim 55; and
(II) collecting die antibody or the functional fragment thereof from the cultures obtained in step (I).

58. The antibody or the functional fragment thereof obtained by a method according to claim 57.

59. A modified form of an antibody or a functional fragment thereof according to claim 35.

60. A pharmaceutical composition comprising an antibody or a functional fragment thereof according to claim 35 as an active ingredient,

61. The pharmaceutical composition according to claim 60, wherein the pharmaceutical composition is a therapeutic or prophylactic drug for autoimmune disease in an individual expressing the polypeptide.

62. The pharmaceutical composition according to claim 61, wherein the autoimmune disease is rheumatoid arthritis.

63. The pharmaceutical composition according to claim 60, wherein the pharmaceutical composition is a therapeutic or prophylactic drug for arthritis in an individual expressing the polypeptide.

64. A method for detecting a polypeptide described in claim 1, comprising the step of contacting a test sample with an antibody that recognizes a polypeptide comprising die amino acid sequence represented by SEQ ID NO: 15 in the Sequence Listing, or a functional fragment thereof, or a modified form of the antibody or the functional fragment.

65. The detection method according to claim 64, wherein the test sample is a test subject-derived sample.

66. The detection method according to claim 65, wherein the test subject-derived sample is plasma.

67. A method for quantifying RX protein, comprising the step of contacting a test sample with an antibody that recognizes a polypeptide comprising the amino acid sequence represented by SEQ ID NO: 15 in the Sequence Listing, or a functional fragment thereof or a modified form of the antibody or die functional fragment.

68. The quantification method according to claim 67, wherein the test sample is a test subject-derived sample.

69. The quantification method according to claim 68, wherein the test subject-derived sample is plasma.

70. The method according to claim 64, wherein the method is performed using an antibody that recognizes a polypeptide comprising the amino acid sequence represented by SEQ ID NO: 15 in the Sequence Listing, or a functional fragment thereof, or a modified form of the antibody or the functional fragment.

71. The method according to claim 64, wherein the method is performed using two or more antibodies that each recognize a polypeptide comprising the amino acid sequence represented by SEQ ID NO: 15 in the Sequence Listing, functional fragments thereof, or modified forms of the antibodies or the functional fragments.

72. The method according to claim 71, wherein the method is performed using sandwich ELISA.

73. A method for examining rheumatoid arthritis, comprising the step of contacting a test sample with an antibody that recognizes a polypeptide comprising the amino acid sequence represented by SEQ ID NO: 15 in the Sequence Listing, or a functional fragment thereof, or a modified form of the antibody or the functional fragment.

74. the examination-method according to claim 73, wherein the test sample is a test subject-derived sample.

75. The examination-method according to claim 74, wherein the test subject-derived sample is plasma.

76. A method for diagnosing rheumatoid arthritis, comprising the following steps (I) to (III):
(I) contacting test subject-derived plasma with an antibody that recognizes a polypeptide comprising the amino acid sequence represented by SEQ ID NO: 15 in the Sequence Listing, or a functional fragment thereof, or a modified form of the antibody or the functional fragment:
(II) determining the amount of a polypeptide described in claim 1 in the test subject-derived plasma; and
(III) diagnosing the test subject as having rheumatoid arthritis or as being at a high risk of acquiring rheumatoid arthritis when the amount of the polypeptide described in step (II) in the test subject-derived plasma is greater than that in healthy individual-derived plasma.

77. A composition for assay of a polypeptide comprising the amino acid sequence represented by SEQ ID NO: 15 in the Sequence Listing or for diagnosis, comprising an antibody that recognizes the polypeptide, or a functional fragment thereof, or a modified form of the antibody or the functional fragment.

78. The composition according to claim 77, wherein the diagnosis is diagnosis of rheumatoid arthritis.

79. The composition according to claim 77, wherein the composition comprises an antibody that recognizes a polypeptide comprising the amino acid sequence represented by SEQ ID NO: 15 in the Sequence Listing, or a functional fragment thereof, or a modified form of the antibody or the functional fragment.

80. The composition according to claim 77, wherein the composition comprises two or more antibodies that each recognize a polypeptide comprising the amino acid sequence represented by SEQ ID NO: 15 in the Sequence Listing, functional fragments thereof or modified forms of the antibodies or the functional fragments.

81. The composition of claim 80, wherein the composition is used ELISA.

82. A reagent or a kit for examination or diagnosis, comprising an antibody that recognizes a polypeptide comprising the amino acid sequence represented by SEQ ID NO: 15 in the Sequence Listing, or a functional fragment thereof, or a modified form of the antibody or the functional fragment.

83. The reagent or the kit according to claim 82, wherein the reagent or the kit is used in the examination or diagnosis of autoimmune disease.

84. The reagent or the kit according to claim 83, wherein the autoimmune disease is rheumatoid arthritis,

85. The reagent or die kit according to claim 82, wherein the reagent or the kit comprises an antibody that recognizes a polypeptide comprising the amino acid sequence represented by SEQ ID NO: 15 in the Sequence Listing, or a functional fragment thereof, or a modified form of the antibody or the functional fragment.

86. The reagent or the kit according to claim 82, wherein the reagent or the kit comprises two or more antibodies that each recognize a polypeptide comprising the amino acid sequence represented by SEQ ID NO: 15 in the Sequence Listing, functional fragments thereof or modified forms of the antibodies or the functional fragments.

87. The reagent or the kit according to claim 82, wherein the reagent or the kit comprises the polypeptide or a fragment thereof or a modified form of the polypeptide or the fragment.

88. The antibody or the functional fragment thereof according to claim 13, wherein the antibody is a rat antibody.

89. The antibody or the functional fragment thereof according to claim 14, wherein the antibody is a mouse antibody.

90. The pharmaceutical composition according to claim 30, wherein the pharmaceutical composition is used in combination with an additional therapeutic or prophylactic agent.


SEQUENCE LISTING (132 sequences)

SEQ ID NO: 1 - protein, 25 aa, Unknown
  Derived from a cell line
ESYWAYLPKPPILHPVGWGSTDPIR

SEQ ID NO: 2 - protein, 17 aa, Unknown
  Derived from a cell line
MWELWLTTLGNSGANTK

SEQ ID NO: 3 - protein, 9 aa, Unknown
  Derived from a cell line
YPHCQIAFK

SEQ ID NO: 4 - protein, 13 aa, Unknown
  Derived from a cell line
DAFWEGDESAPPR

SEQ ID NO: 5 - protein, 23 aa, Unknown
  Derived from a cell line
GALGLLWDFSLPSPSVDQSDQIK

SEQ ID NO: 6 - protein, 18 aa, Unknown
  Derived from a cell line
WYEAGWVEPTWFWENSPK

SEQ ID NO: 7 - protein, 18 aa, Unknown
  Derived from a cell line
DPNDRDFTALVPHTELFR

SEQ ID NO: 8 - protein, 13 aa, Unknown
  Derived from a cell line
DFTALVPHTELFR

SEQ ID NO: 9 - protein, 34 aa, Unknown
  Derived from a cell line
RPGFQEHEMIPTSACVTYPYAILLGLPQLIDIEK

SEQ ID NO: 10 - protein, 12 aa, Unknown
  Derived from a cell line
GSTFHISCSSCR

SEQ ID NO: 11 - protein, 18 aa, Unknown
  Derived from a cell line
LTNCLDSSAYDYAAIIVK

SEQ ID NO: 12 - protein, 26 aa, Unknown
  Derived from a cell line
RPPYVLLPVDIGDEPWFDDSAIQTFR

SEQ ID NO: 13 - protein, 7 aa, Unknown
  Derived from a cell line
YATDLIR

SEQ ID NO: 14 - DNA, 1662 bp, Unknown
  Derived from a cell line
gaaagttattgggcttacctacctaaaccacctattctccatcccgtgggatggggaagtacagaccccattagagttctgaccaatcaaaccatgtatttgggtgggtcgcctgactttcacgggtttagaaacatgtctggcaatgtacattttgaggggaagtctgatacgctccccatttgcttttccttctccttttctacccccacgggctgctttcaagtagataagcaagtatttctttctgatacacccacggttgataataataaacctgggggaaagggtgataaaaggcgtatgtgggaactttggttgactactttggggaactcaggggccaatacaaaactggtccctataaaaaagaagttgccccccaaatatcctcactgccagatcgcctttaagaaggacgccttctgggagggagacgagtctgctcctccacggtggttgccttgcgccttccctgaccagggggtgagtttttctccaagaggggcccttgggttactttgggatttctcccttccctcgcctagtgtagatcagtcagatcagattaaaagcaaaaaggatctctttggaaattatactccccctgtcaataaagaggttcatcgatggtatgaagcaggatgggtagaacctacatggttctgggaaaattctcctaaggatcccaatgatagagattttactgctctagttccccatacagaattgtttcgcttagttgcagcctcaagacatcttattctcaaaaggccaggatttcaagaacatgaaatgattcctacatctgcctgtgttacttacccttatgccatattattaggattacctcagctaatagatatagagaaaagaggatctacttttcatatttcctgttcttcttgtagattgactaattgtttagattcttctgcctacgactatgcagcgatcatagtcaagaggccgccatacgtgctgctacctgtagatattggtgatgaaccatggtttgatgattctgccattcaaacctttaggtatgccacagatttaattcgagctaagcgattcgtcgctgccattattctgggcatatctgctttaattgctattatcacttcctttgctgtagctactactgctttagttaaggagatgcaaactgctacgtttgttaataatcttcatagaaatgttacattagccttatctgaacaaagaataatagatttaaaattagaagctagacttaatgctttagaagaagtagttttagagttgggacaagatgtggcaaacttaaagaccagaatgtccaccaggtgtcatgcaaattatgattttatctgcgttacacctttaccatataatgcttctgagagctgggaaagaaccaaagctcatttattgggcatttggaatgacaatgagatttcatataacatacaagaattaaccaacctgattagtgatatgagcaaacaacatattgacgcagtggacctcggtggcttggctcagtcctttgccaatggagtaaaggctttaaatccattagattggacacaatatttcatttttataggtgttagagccctgcttttagtcatagtgcttatgattttccccattgttttccagtgctttgcgaagtag

SEQ ID NO: 15 - protein, 553 aa, Unknown
  Synthetic Construct
ESYWAYLPKPPILHPVGWGSTDPIRVLTNQTMYLGGSPDFHGFRNMSGNVHFEGKSDTLPICFSFSFSTPTGCFQVDKQVFLSDTPTVDNNKPGGKGDKRRMWELWLTTLGNSGANTKLVPIKKKLPPKYPHCQIAFKKDAFWEGDESAPPRWLPCAFPDQGVSFSPRGALGLLWDFSLPSPSVDQSDQIKSKKDLFGNYTPPVNKEVHRWYEAGWVEPTWFWENSPKDPNDRDFTALVPHTELFRLVAASRHLILKRPGFQEHEMIPTSACVTYPYAILLGLPQLIDIEKRGSTFHISCSSCRLTNCLDSSAYDYAAIIVKRPPYVLLPVDIGDEPWFDDSAIQTFRYATDLIRAKRFVAAIILGISALIAIITSFAVATTALVKEMQTATFVNNLHRNVTLALSEQRIIDLKLEARLNALEEVVLELGQDVANLKTRMSTRCHANYDFICVTPLPYNASESWERTKAHLLGIWNDNEISYNIQELTNLISDMSKQHIDAVDLGGLAQSFANGVKALNPLDWTQYFIFIGVRALLLVIVLMIFPIVFQCFAK

SEQ ID NO: 16 - DNA, 25 bp, Artificial
  Synthetic oligonucleotide
tcatttacccggagagtgggagaga

SEQ ID NO: 17 - DNA, 25 bp, Artificial
  Synthetic oligonucleotide
ctaacactcattcctgttgaagctc

SEQ ID NO: 18 - DNA, 1383 bp, Rattus norvegicus
  CDS (1)..(1383)
atggacaccaggctcagcttggttttcattgtccttttcataaaaggtgtccagtgtgaggtgcagctggtggagtctgggggaggcttagtgcagcctggaaggtccctgaaactctcctgtgtaggttcaggattcactttcagtgactattacatggcctgggtccgccaggctccaaagaagggtctggagtgggtcgcatccattagttatgagggtagtaacactaactttggagactccgtgaagggccgattcactatctccagagatagtgcaaaaagcaccctatacctacaaatgaacagtctgaggtctgaggacacggccacttattattgtgcaagatgggcccaaacagggtactactttgattactggggccaaggagtcatggtcacagtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtagcccacccggccagcagcaccaaggtggacaagaaaattgtgccaagggaatgcaatccttgtggatgtacaggctcagaagtatcatctgtcttcatcttccccccaaagaccaaagatgtgctcaccatcactctgactcctaaggtcacgtgtgttgtggtagacattagccagaatgatcccgaggtccggttcagctggtttatagatgacgtggaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaaagcatctccaaacccgaaggcacaccacgaggtccacaggtatacaccatggcgcctcccaaggaagagatgacccagagtcaagtcagtatcacctgcatggtaaaaggcttctatcccccagacatttatacggagtggaagatgaacgggcagccacaggaaaactacaagaacactccacctacgatggacacagatgggagttacttcctctacagcaagctcaatgtaaagaaagaaacatggcagcagggaaacactttcacgtgttctgtgctgcatgagggcctgcacaaccaccatactgagaagagtctctcccactctccgggtaaataa

SEQ ID NO: 19 - protein, 460 aa, Rattus norvegicus
MDTRLSLVFIVLFIKGVQCEVQLVESGGGLVQPGRSLKLSCVGSGFTFSDYYMAWVRQAPKKGLEWVASISYEGSNTNFGDSVKGRFTISRDSAKSTLYLQMNSLRSEDTATYYCARWAQTGYYFDYWGQGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETWQQGNTFTCSVLHEGLHNHHTEKSLSHSPGK

SEQ ID NO: 20 - DNA, 705 bp, Rattus norvegicus
  CDS (1)..(705)
atggctgtgcccactcagctcctggggttgttgctgctgtggattacagatgccatatgtgacattcagatgacacagtctccagcttccctgtctgcatctctgggagaaactgtcaccatcgaatgtctagcaagtgaggatatttacagtaatttagcttggtatcagcagaagccagggaaatctcctcagctcctgatctattatgcaaatagcttgaatgatggcgtcccatcacggttcagtggcagtggatctggcacacagttttctctgaagatcaacagcctgcaatctgaagatgtctcgatttatttctgtcaacagaattatgatagtccgtacacgtttggagctggggccaagctggaactgaaacgggctgatgctgcaccaactgtatctatcttcccaccatccacgaaacagttagcaactggaggtgcctcagtcgtgtgcctcatgaacaacttctatcccagagacatcagtgtcaagtggaagattgatggcactgaacgacgagatggtgtcctggacagtgttactgatcaggacagcaaagacagcacgtacagcatgagcagcaccctctcgttgaccaaggctgactatgaaagtcataacctctatacctgtgaggttgttcataagacatcatcctcacccgtcgtcaagagcttcaacaggaatgagtgttaa

SEQ ID NO: 21 - protein, 234 aa, Rattus norvegicus
MAVPTQLLGLLLLWITDAICDIQMTQSPASLSASLGETVTIECLASEDIYSNLAWYQQKPGKSPQLLIYYANSLNDGVPSRFSGSGSGTQFSLKINSLQSEDVSIYFCQQNYDSPYTFGAGAKLELKRADAAPTVSIFPPSTKQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 22 - protein, 5 aa, Rattus norvegicus
DYYMA

SEQ ID NO: 23 - protein, 17 aa, Rattus norvegicus
SISYEGSNTNFGDSVKG

SEQ ID NO: 24 - protein, 10 aa, Rattus norvegicus
WAQTGYYFDY

SEQ ID NO: 25 - protein, 11 aa, Rattus norvegicus
LASEDIYSNLA

SEQ ID NO: 26 - protein, 7 aa, Rattus norvegicus
YANSLND

SEQ ID NO: 27 - protein, 8 aa, Rattus norvegicus
QQNYDSPY

SEQ ID NO: 28 - DNA, 31 bp, Artificial
  Synthetic oligonucleotide
ccgctagcatgsargtnmagctgsagsagtc

SEQ ID NO: 29 - DNA, 29 bp, Artificial
  Synthetic oligonucleotide
agcgctcttgaccaggcatcctagagtca

SEQ ID NO: 30 - DNA, 31 bp, Artificial
  Synthetic oligonucleotide
ccccatggayattgtgmtsacmcarwctmca

SEQ ID NO: 31 - DNA, 39 bp, Artificial
  Synthetic oligonucleotide
ccctcgagttcaacactcattcctgttgaagccttgacg

SEQ ID NO: 32 - DNA, 339 bp, Mus musculus
  CDS (1)..(339)
gaggtgaagctgcaggagtctggacctgagctgaagaagcctggagagacagtcaagatctcctgcaaggcttctggttataccttcagagactatccaattcactgggtgaagcaggctccaggaaagggtttaaagtggatgggctggataaacactgagactggtgagccgacatatgcggatgacttcaagggacgctttgccttctctttggaaacctctgccaacactacctatttgcagatcaacaacctcaaaaatgaggacacggctacatatttctgtactcccaggtttacttactggggccaagggactctggtcactgtctcttca

SEQ ID NO: 33 - protein, 113 aa, Mus musculus
EVKLQESGPELKKPGETVKISCKASGYTFRDYPIHWVKQAPGKGLKWMGWINTETGEPTYADDFKGRFAFSLETSANTTYLQINNLKNEDTATYFCTPRFTYWGQGTLVTVSS

SEQ ID NO: 34 - DNA, 342 bp, Mus musculus
  CDS (1)..(342)
gatattgtgatgacacagactccatcctccctagctgtgtcagttggagagaaggttactatgagctgcaagtccagtcagaaccttttatatagtggcaatcaaaagaactacttggcctggttccagcagaaaccagggcagtctcctaaactgctgatttactgggcatccactagggaatctggggtccctgatcgcttcacaggcagtgggtctgggacagatttcactctcactctcagcagtgtgaaggctgaagacctggcagtttattactgtcagcaatcttataggtatccgtatacgtccggaggggggaccaagctggaaataaaacgg

SEQ ID NO: 35 - protein, 114 aa, Mus musculus
DIVMTQTPSSLAVSVGEKVTMSCKSSQNLLYSGNQKNYLAWFQQKPGQSPKLLIYWASTRESGVPDRFTGSGSGTDFTLTLSSVKAEDLAVYYCQQSYRYPYTSGGGTKLEIKR

SEQ ID NO: 36 - protein, 5 aa, Mus musculus
DYPIH

SEQ ID NO: 37 - protein, 17 aa, Mus musculus
WINTETGEPTYADDFKG

SEQ ID NO: 38 - protein, 4 aa, Mus musculus
RFTY

SEQ ID NO: 39 - protein, 17 aa, Mus musculus
KSSQNLLYSGNQKNYLA

SEQ ID NO: 40 - protein, 7 aa, Mus musculus
WASTRES

SEQ ID NO: 41 - protein, 8 aa, Mus musculus
QQSYRYPY

SEQ ID NO: 42 - protein, 25 aa, Unknown
  Derived from a subject
ESYWAYLPKPPILHPVGWGSTDPIR

SEQ ID NO: 43 - protein, 18 aa, Unknown
  Derived from a subject
QVFLSDTPTVDNNKPGGK

SEQ ID NO: 44 - protein, 17 aa, Unknown
  Derived from a subject
MWELWLTTLGNSGANTK

SEQ ID NO: 45 - protein, 14 aa, Unknown
  Derived from a subject
KDAFWEGDESAPPR

SEQ ID NO: 46 - protein, 13 aa, Unknown
  Derived from a subject
DAFWEGDESAPPR

SEQ ID NO: 47 - protein, 23 aa, Unknown
  Derived from a subject
GALGLLWDFSLPSPSVDQSDQIK

SEQ ID NO: 48 - protein, 18 aa, Unknown
  Derived from a subject
WYEAGWVEPTWFWENSPK

SEQ ID NO: 49 - protein, 18 aa, Unknown
  Derived from a subject
DPNDRDFTALVPHTELFR

SEQ ID NO: 50 - protein, 13 aa, Unknown
  Derived from a subject
DFTALVPHTELFR

SEQ ID NO: 51 - protein, 12 aa, Unknown
  Derived from a subject
GSTFHISCSSCR

SEQ ID NO: 52 - protein, 18 aa, Unknown
  Derived from a subject
LTNCLDSSAYDYAAIIVK

SEQ ID NO: 53 - DNA, 19 bp, Artificial
  Synthetic oligonucleotide
ccagatcgcctttaagaag

SEQ ID NO: 54 - DNA, 26 bp, Artificial
  Synthetic oligonucleotide
ctatcattgggatccttaggagaatt

SEQ ID NO: 55 - DNA, 21 bp, Artificial
  Synthetic oligonucleotide
tgcgccttccctgaccaaggg

SEQ ID NO: 56 - protein, 100 aa, Unknown
  Derived from a subject
QIAFKKDAFWEGDESAPPRWLPCAFPDQGVSFSPRGALGLLWDFSLPSPSVDQSDQIKSKKDLFGNYTPPVNKEVHRWYEAGWVEPTWFWENSPKDPNDR

SEQ ID NO: 57 - protein, 100 aa, Unknown
  Derived from a subject
QIAFKKDAFWEGDESAPPRWLPCAFPDQGVSFSPKGALGLLWDFSLPSPSVDQSDQIKNKKDLFGNYTPPVNKEVHRWYEAGWVEPTWFWENSPKDPNDR

SEQ ID NO: 58 - protein, 100 aa, Unknown
  Derived from a subject
QIAFKKDAFWEGDESAPPRWLPCAFPDQGVSFSPKGSLGLLWDFSLPSPSVDQSDQIKNKKDLFGNYTPPVNKEVHRWYEAGWVEPTWFWENSPKDPNDR

SEQ ID NO: 59 - protein, 100 aa, Unknown
  Derived from a subject
QIAFKKDAFWEGDESAPPRWLPCAFPDQGVSFSPKGALGLLWDFSLPSPSIDQSEQIKSKKDLLGNYTPPVNKEVHRWYEAGWVEPTWFWENSPKDPNDR

SEQ ID NO: 60 - DNA, 2067 bp, Unknown
  Derived from a cell line
atgccgaatcaccaatctgggtccccgaccggttcatccgaccttttactgagcggaaagaagcaacgcccacacctggcactgcggagaaaacgccgccgcgagatgagaaagatcaacaggaaagtccggaggatgaatatagcccccatcaaagagaagacggcttggcaacatctgcaggcgttaatctccgaagcggaggaggttcttaaaacctcacaaactccccaaacctctttgactttatttcttgctttgttgtctgtcctcggccccccgcctgtggccggggaaagttattgggcttacctacctaaaccacctattctccatcccgtgggatggggaagtacagaccccattagagttctgaccaatcaaaccatgtatttgggtgggtcgcctgactttcacgggtttagaaacatgtctggcaatgtacattttgaggggaagtctgatacgctccccatttgcttttccttctccttttctacccccacgggctgctttcaagtagataagcaagtatttctttctgatacacccacggttgataataataaacctgggggaaagggtgataaaaggcgtatgtgggaactttggttgactactttggggaactcaggggccaatacaaaactggtccctataaaaaagaagttgccccccaaatatcctcactgccagatcgcctttaagaaggacgccttctgggagggagacgagtctgctcctccacggtggttgccttgcgccttccctgaccagggggtgagtttttctccaagaggggcccttgggttactttgggatttctcccttccctcgcctagtgtagatcagtcagatcagattaaaagcaaaaaggatctctttggaaattatactccccctgtcaataaagaggttcatcgatggtatgaagcaggatgggtagaacctacatggttctgggaaaattctcctaaggatcccaatgatagagattttactgctctagttccccatacagaattgtttcgcttagttgcagcctcaagacatcttattctcaaaaggccaggatttcaagaacatgaaatgattcctacatctgcctgtgttacttacccttatgccatattattaggattacctcagctaatagatatagagaaaagaggatctacttttcatatttcctgttcttcttgtagattgactaattgtttagattcttctgcctacgactatgcagcgatcatagtcaagaggccgccatacgtgctgctacctgtagatattggtgatgaaccatggtttgatgattctgccattcaaacctttaggtatgccacagatttaattcgagctaagcgattcgtcgctgccattattctgggcatatctgctttaattgctattatcacttcctttgctgtagctactactgctttagttaaggagatgcaaactgctacgtttgttaataatcttcatagaaatgttacattagccttatctgaacaaagaataatagatttaaaattagaagctagacttaatgctttagaagaagtagttttagagttgggacaagatgtggcaaacttaaagaccagaatgtccaccaggtgtcatgcaaattatgattttatctgcgttacacctttaccatataatgcttctgagagctgggaaagaaccaaagctcatttattgggcatttggaatgacaatgagatttcatataacatacaagaattaaccaacctgattagtgatatgagcaaacaacatattgacgcagtggacctcggtggcttggctcagtcctttgccaatggagtaaaggctttaaatccattagattggacacaatatttcatttttataggtgttagagccctgcttttagtcatagtgcttatgattttccccattgttttccagtgctttgcgaagagccttgaccaagtgcagtcagatcttaacgtgcttcttttaaaaaagaaaaaagggggaaatgccgcgcctgcagcagaaatggttgaactcccgagagtgtcctacccttag

SEQ ID NO: 61 - protein, 688 aa, Unknown
  Synthetic Construct
MPNHQSGSPTGSSDLLLSGKKQRPHLALRRKRRREMRKINRKVRRMNIAPIKEKTAWQHLQALISEAEEVLKTSQTPQTSLTLFLALLSVLGPPPVAGESYWAYLPKPPILHPVGWGSTDPIRVLTNQTMYLGGSPDFHGFRNMSGNVHFEGKSDTLPICFSFSFSTPTGCFQVDKQVFLSDTPTVDNNKPGGKGDKRRMWELWLTTLGNSGANTKLVPIKKKLPPKYPHCQIAFKKDAFWEGDESAPPRWLPCAFPDQGVSFSPRGALGLLWDFSLPSPSVDQSDQIKSKKDLFGNYTPPVNKEVHRWYEAGWVEPTWFWENSPKDPNDRDFTALVPHTELFRLVAASRHLILKRPGFQEHEMIPTSACVTYPYAILLGLPQLIDIEKRGSTFHISCSSCRLTNCLDSSAYDYAAIIVKRPPYVLLPVDIGDEPWFDDSAIQTFRYATDLIRAKRFVAAIILGISALIAIITSFAVATTALVKEMQTATFVNNLHRNVTLALSEQRIIDLKLEARLNALEEVVLELGQDVANLKTRMSTRCHANYDFICVTPLPYNASESWERTKAHLLGIWNDNEISYNIQELTNLISDMSKQHIDAVDLGGLAQSFANGVKALNPLDWTQYFIFIGVRALLLVIVLMIFPIVFQCFAKSLDQVQSDLNVLLLKKKKGGNAAPAAEMVELPRVSYP

SEQ ID NO: 62 - DNA, 363 bp, Mus musculus
  CDS (1)..(363)
caggtgaagctggaggagtctgggggaggcttagtgaagcctggagggtccctgaaactctcctgtgcagcctctggattcagttttagtagaaatgtcatgtcttgggttcgccagactccggagaagaggctggagtgggtcgcaaccattagtgctggtggtagttacacctattatccagacagtgtgaaggggcgattcaccatctccagagacaatgccaagaacaccctgtatctgcaaatgagcagtctgaggtctgaggacacggccatgtattactgtgcaagacttaattataacgataacgggaactactttgactactggggccaaggcaccactctcacagtctcctca

SEQ ID NO: 63 - protein, 121 aa, Mus musculus
QVKLEESGGGLVKPGGSLKLSCAASGFSFSRNVMSWVRQTPEKRLEWVATISAGGSYTYYPDSVKGRFTISRDNAKNTLYLQMSSLRSEDTAMYYCARLNYNDNGNYFDYWGQGTTLTVSS

SEQ ID NO: 64 - DNA, 321 bp, Mus musculus
  CDS (1)..(321)
gatattgtgatgacacagtctacagcaatcatgtctgcatctctaggggagaaggtcaccatgagctgcagggccagctcaagtgtagtttacatgtactggtaccagcagaagtcagatgcctcccccaaactatggatttattacacatccaacctggctcctggagtcccggctcgcttcagtggcagtgggtctgggaactcttattctctcacaatcagcagcatggagggtgaagatgctgccacttattactgccagcagtttactagttccccatacacgttcggaggggggaccaagctggaaataaaacgg

SEQ ID NO: 65 - protein, 107 aa, Mus musculus
DIVMTQSTAIMSASLGEKVTMSCRASSSVVYMYWYQQKSDASPKLWIYYTSNLAPGVPARFSGSGSGNSYSLTISSMEGEDAATYYCQQFTSSPYTFGGGTKLEIKR

SEQ ID NO: 66 - protein, 5 aa, Mus musculus
RNVMS

SEQ ID NO: 67 - protein, 17 aa, Mus musculus
TISAGGSYTYYPDSVKG

SEQ ID NO: 68 - protein, 12 aa, Mus musculus
LNYNDNGNYFDY

SEQ ID NO: 69 - protein, 10 aa, Mus musculus
RASSSVVYMY

SEQ ID NO: 70 - protein, 7 aa, Mus musculus
YTSNLAP

SEQ ID NO: 71 - protein, 8 aa, Mus musculus
QQFTSSPY

SEQ ID NO: 72 - protein, 119 aa, Artificial
  Synthetic peptide
EVQLVESGGGLVQPGRSLRLSCAASGFTFSDYYMAWVRQAPGKGLEWVSSISYEGSNTNFGDSVKGRFTISRDNSKNTLYLKMNSLRAEDTAVYYCARWAQTGYYFDYWGQGTLVTVSS

SEQ ID NO: 73 - protein, 119 aa, Artificial
  Synthetic peptide
EVQLVESGGGLVQPGRSLRLSCAASGFTFSDYYMAWVRQAPGKGLEWVASISYEGSNTNFGDSVKGRFTISRDNSKNTLYLKMNSLRAEDTAVYYCARWAQTGYYFDYWGQGTLVTVSS

SEQ ID NO: 74 - protein, 119 aa, Artificial
  synthetic peptide
EVQLVESGGGLVQPGRSLRLSCAGSGFTFSDYYMAWVRQAPGKGLEWVASISYEGSNTNFGDSVKGRFTISRDSSKNTLYLKMNSLRAEDTAVYYCARWAQTGYYFDYWGQGTLVTVSS

SEQ ID NO: 75 - protein, 119 aa, Artificial
  synthetic peptide
EVQLVESGGGLVQPGRSLRLSCAGSGFTFSDYYMAWVRQAPGKGLEWVASISYEGSNTNFGDSVKGRFTISRDSAKNTLYLKMNSLRAEDTAVYYCARWAQTGYYFDYWGQGTLVTVSS

SEQ ID NO: 76 - protein, 119 aa, Artificial
  synthetic peptide
EVQLVESGGGLVQPGRSLRLSCAASGFTFSDYYMAWVRQAPGKGLEWVASISYEGSNTNFGDSVKGRFTISRDSSKNTLYLKMNSLRAEDTAVYYCARWAQTGYYFDYWGQGTLVTVSS

SEQ ID NO: 77 - protein, 119 aa, Artificial
  Synthetic peptide
EVQLVESGGGLVQPGRSLRLSCAGSGFTFSDYYMAWVRQAPGKGLEWVASISYEGSNTNFGDSVKGRFTISRDNSKNTLYLKMNSLRAEDTAVYYCARWAQTGYYFDYWGQGTLVTVSS

SEQ ID NO: 78 - protein, 119 aa, Artificial
  Synthetic peptide
EVQLVESGGGLVQPGRSLRLSCAGSGFTFSDYYMAWVRQAPGKGLEWVASISYEGSNTNFGDSVKGRFTISRDSSKNTLYLQMNSLRAEDTAVYYCARWAQTGYYFDYWGQGTLVTVSS

SEQ ID NO: 79 - protein, 119 aa, Artificial
  Synthetic peptide
EVQLVESGGGLVQPGRSLRLSCAASGFTFSDYYMAWVRQAPGKGLEWVSSISYEGSNTNFGDSVKGRFTISRDSSKNTLYLKMNSLRAEDTAVYYCARWAQTGYYFDYWGQGTLVTVSS

SEQ ID NO: 80 - protein, 119 aa, Artificial
  Synthetic peptide
EVQLVESGGGLVKPGGSLRLSCAASGFTFSDYYMAWVRQAPGKGLEWVASISYEGSNTNFGDSVKGRFTISRDSSKNTLYLKMNSLRAEDTAVYYCARWAQTGYYFDYWGQGTLVTVSS

SEQ ID NO: 81 - protein, 119 aa, Artificial
  synthetic peptide
EVQLVESGGGLVKPGGSLRLSCAASGFTFSDYYMAWVRQAPGKGLEWVSSISYEGSNTNFGDSVKGRFTISRDSSKNTLYLKMNSLRAEDTAVYYCARWAQTGYYFDYWGQGTLVTVSS

SEQ ID NO: 82 - protein, 109 aa, Artificial
  Synthetic peptide
DIQMTQSPSSLSASVGDRVTITCLASEDIYSNLAWYQQKPGKAPKLLIYYANSLNDGVPSRFSGSGSGTDFTLTISRLQPEDFATYYCQQNYDSPYTFGQGTKVEIKRT

SEQ ID NO: 83 - protein, 109 aa, Artificial
  synthetic peptide
DIQMTQSPSSLSASVGDRVTITCLASEDIYSNLAWYQQKPGKSPKLLIYYANSLNDGVPSRFSGSGSGTDFTLTISRLQPEDFATYYCQQNYDSPYTFGQGTKVEIKRT

SEQ ID NO: 84 - protein, 109 aa, Artificial
  Synthetic peptide
DIQMTQSPSSLSASVGDRVTITCLASEDIYSNLAWYQQKPGKSPQLLIYYANSLNDGVPSRFSGSGSGTDFTLTISRLQPEDFATYFCQQNYDSPYTFGQGTKVEIKRT

SEQ ID NO: 85 - protein, 109 aa, Artificial
  Synthetic peptide
DIQMTQSPSSLSASVGDRVTITCLASEDIYSNLAWYQQKPGKSPQLLIYYANSLNDGVPSRFSGSGSGTDFTLTISRLQPEDFATYFCQQNYDSPYTFGQGTKLEIKRT

SEQ ID NO: 86 - protein, 109 aa, Artificial
  Synthetic peptide
DIQMTQSPSSLSASVGDRVTITCLASEDIYSNLAWYQQKPGKSPKLLIYYANSLNDGVPSRFSGSGSGTDFTLTISSLQPEDFATYYCQQNYDSPYTFGQGTKVEIKRT

SEQ ID NO: 87 - DNA, 993 bp, Homo sapiens
  CDS (1)..(993)
gccagcacaaagggcccctccgtgtttccattggccccgtcttcaaaaagcacatctggcggtaccgctgctctcggctgtcttgttaaggactactttcccgaacccgtgacggtgtcctggaactccggtgccctgacctcaggcgtgcataccttcccggctgttctccagtcatcaggcctgtattcactgtcaagcgttgtgactgtcccgtcatctagtttgggcacacagacatatatctgtaacgtcaatcacaagccatcaaacaccaaagtcgacaagcgggtggaacctaagagttgcgacaagactcacacttgcccaccatgccctgctcccgagctgctgggagggccatctgttttcctgtttcctcccaagccaaaagatacactgatgatcagccggacccccgaagtaacttgcgtagtagtcgacgtgtctcacgaagatcccgaggtcaaatttaactggtacgtggatggcgttgaggtacacaacgccaagacaaaacccagggaagaacaatacaattcaacttatcgggtcgtctccgtccttacggtgctgcatcaggattggctgaacggcaaagaatacaagtgtaaggttagcaacaaggctcttcctgcccctattgagaagactatctctaaggcaaagggacagccacgggaacctcaggtgtacaccttgccaccgagtcgggaagaaatgaccaaaaatcaggtttccctgacctgccttgtgaaaggtttttatcccagcgacattgccgtagagtgggagtccaatggtcagcccgagaacaactacaaaaccactccccctgttctggacagcgatgggagcttttttctctacagcaagctgaccgtggataagagtcgctggcagcagggcaacgtgttcagctgtagcgtgatgcacgaggccctgcataatcattacacacaaaagagcctgtccctcagccccgggaagtga

SEQ ID NO: 88 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 93 bp, Artificial
  Synthetic oligonucleotide
gaattcgccaccatgaagttcagctgggtgatgttcttcctgatggccgtggtgaccggcgtgaacagcgctagcacaaagggcccctccgtg

SEQ ID NO: 90 - DNA, 30 bp, Artificial
  Synthetic oligonucleotide
ctcgagtcacttcccggggctgagggacag

SEQ ID NO: 91 - DNA, 357 bp, Artificial
  Synthetic nucleotide
gaagtgcagctcgtggaatcaggaggcgggttggtgcagccaggtcgcagtctgaggttgagctgcgccgcctctgggttcaccttttcagattactacatggcctgggtgaggcaggcacccggcaaaggacttgagtgggtgtcttcaatttcttacgagggcagtaataccaatttcggcgactctgttaagggacggttcacaataagtcgggataatagcaaaaacactctgtatctgaaaatgaatagtctcagggcagaggatacagccgtgtactattgcgccaggtgggcccagaccggatactacttcgactactggggccagggcaccctggtgaccgtgagcagc

SEQ ID NO: 92 - protein, 119 aa, Artificial; identical to SEQ ID NO: 72
  Synthetic Construct

SEQ ID NO: 93 - DNA, 357 bp, Artificial
  Synthetic nucleotide
gaggtccagttggtggagagcggggggggcctcgttcagcccgggcggagtctccgcctgtcatgcgctgccagcggattcacattcagtgactactatatggcctgggtgcgccaggctccaggcaaaggtttggaatgggtggcaagtattagttacgagggaagcaacaccaattttggcgatagtgtgaagggtcggtttaccatctcccgcgataacagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 94 - protein, 119 aa, Artificial; identical to SEQ ID NO: 73
  Synthetic Construct

SEQ ID NO: 95 - DNA, 357 bp, Artificial
  Synthetic nucleotide
gaggtgcagctggtggaatcaggagggggtctggtccagcctgggaggagcctgcggctgtcttgcgccggctccggtttcacatttagtgattactacatggcatgggtgagacaagcccctggaaaaggactggagtgggttgcttccatctcatacgagggcagcaacacaaactttggggattccgtcaaagggagattcacaatctcaagggatagcagtaaaaataccctgtaccttaagatgaacagcttgcgcgctgaggacaccgccgtgtactattgcgccagatgggcccagacaggctactacttcgactactggggccagggcaccctggtgaccgtgagcagc

SEQ ID NO: 96 - protein, 119 aa, Artificial; identical to SEQ ID NO: 74
  Synthetic Construct

SEQ ID NO: 97 - DNA, 357 bp, Artificial
  Synthetic nucleotide
gaagtgcagctcgttgaatccggaggagggctcgtgcagcctggccgcagtctcaggctcagctgcgctggttccggcttcaccttctccgactactacatggcatgggtgcggcaggctccagggaagggactggaatgggttgcctctatttcctatgagggctccaataccaacttcggcgactccgtgaaagggcgctttacaatttcaagggattcagctaaaaacaccctgtatctcaaaatgaacagcctgagggcagaagacaccgctgtttattattgcgccagatgggcccagaccggctactacttcgactactggggccagggcaccctggtgaccgtgagcagc

SEQ ID NO: 98 - protein, 119 aa, Artificial; identical to SEQ ID NO: 75
  Synthetic Construct

SEQ ID NO: 99 - DNA, 357 bp, Artificial
  Synthetic nucleotide
gaggtccagttggtggagagcggggggggcctcgttcagcccgggcggagtctccgcctgtcatgcgctgccagcggattcacattcagtgactactatatggcctgggtgcgccaggctccaggcaaaggtttggaatgggtggcaagtattagttacgagggaagcaacaccaattttggcgatagtgtgaagggtcggtttaccatctcccgcgatagcagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 100 - protein, 119 aa, Artificial; identical to SEQ ID NO: 76
  Synthetic Construct

SEQ ID NO: 101 - DNA, 357 bp, Artificial
  Synthetic nucleotide
gaggtccagttggtggagagcggggggggcctcgttcagcccgggcggagtctccgcctgtcatgcgctggcagcggattcacattcagtgactactatatggcctgggtgcgccaggctccaggcaaaggtttggaatgggtggcaagtattagttacgagggaagcaacaccaattttggcgatagtgtgaagggtcggtttaccatctcccgcgataacagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 102 - protein, 119 aa, Artificial; identical to SEQ ID NO: 77
  Synthetic Construct

SEQ ID NO: 103 - DNA, 357 bp, Artificial
  Synthetic nucleotide
gaggtgcagctggtggaatcaggagggggtctggtccagcctgggaggagcctgcggctgtcttgcgccggctccggtttcacatttagtgattactacatggcatgggtgagacaagcccctggaaaaggactggagtgggttgcttccatctcatacgagggcagcaacacaaactttggggattccgtcaaagggagattcacaatctcaagggatagcagtaaaaataccctgtaccttcagatgaacagcttgcgcgctgaggacaccgccgtgtactattgcgccagatgggcccagacaggctactacttcgactactggggccagggcaccctggtgaccgtgagcagc

SEQ ID NO: 104 - protein, 119 aa, Artificial; identical to SEQ ID NO: 78
  Synthetic Construct

SEQ ID NO: 105 - DNA, 357 bp, Artificial
  Synthetic nucleotide
gaggtccagttggtggagagcggggggggcctcgttcagcccgggcggagtctccgcctgtcatgcgctgccagcggattcacattcagtgactactatatggcctgggtgcgccaggctccaggcaaaggtttggaatgggtgtcaagtattagttacgagggaagcaacaccaattttggcgatagtgtgaagggtcggtttaccatctcccgcgatagcagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 106 - protein, 119 aa, Artificial; identical to SEQ ID NO: 79
  Synthetic Construct

SEQ ID NO: 107 - DNA, 357 bp, Artificial
  Synthetic nucleotide
gaggtccagttggtggagagcggggggggcctcgttaagcccggggggagtctccgcctgtcatgcgctgccagcggattcacattcagtgactactatatggcctgggtgcgccaggctccaggcaaaggtttggaatgggtggcaagtattagttacgagggaagcaacaccaattttggcgatagtgtgaagggtcggtttaccatctcccgcgatagcagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 108 - protein, 119 aa, Artificial; identical to SEQ ID NO: 80
  Synthetic Construct

SEQ ID NO: 109 - DNA, 357 bp, Artificial
  Synthetic nucleotide
gaggtccagttggtggagagcggggggggcctcgttaagcccggggggagtctccgcctgtcatgcgctgccagcggattcacattcagtgactactatatggcctgggtgcgccaggctccaggcaaaggtttggaatgggtgtcaagtattagttacgagggaagcaacaccaattttggcgatagtgtgaagggtcggtttaccatctcccgcgatagcagtaagaatacactgtacttgaaaatgaactcccttcgggctgaagatacagcagtgtattattgtgcccggtgggcccagaccggatattacttcgactactggggccagggcaccctggtgaccgtgagcagc

SEQ ID NO: 110 - protein, 119 aa, Artificial; identical to SEQ ID NO: 81
  Synthetic Construct

SEQ ID NO: 111 - DNA, 318 bp, Homo sapiens
  CDS (1)..(318)
gtggccgccccatccgtgtttatctttccccccagtgatgagcagttgaagtcagggactgctagtgtggtctgtttgttgaacaacttctacccccgcgaggccaaggtccagtggaaggtagataacgccctgcaatcagggaatagtcaggagtctgtgaccgaacaggactccaaggatagcacttatagcttgagctccaccctgaccctgagtaaggctgactacgaaaaacacaaggtgtacgcatgtgaagttacccatcaggggctgagttcccccgtaactaaatcttttaatcgaggcgaatgctga

SEQ ID NO: 112 - protein, 105 aa, Homo sapiens [sequence omitted]

SEQ ID NO: 113 - DNA, 327 bp, Artificial
  Synthetic nucleotide
gatattcagatgactcagagtccatccagtctcagcgcttccgttggcgatagggtcacaatcacctgcctcgcttcagaggatatctattccaatctggcctggtatcagcagaagcctgggaaagcccccaaactgttgatctactatgcaaattccctgaacgacggggtgccttcaagatttagtgggagtggatcaggcaccgattttaccttgaccatctcccggcttcagccagaggatttcgcaacttactactgtcagcagaattatgactccccttacaccttcggacagggcaccaaggtggagattaagcggacc

SEQ ID NO: 114 - protein, 109 aa, Artificial; identical to SEQ ID NO: 82
  Synthetic Construct

SEQ ID NO: 115 - DNA, 327 bp, Artificial
  Synthetic nucleotide
gatattcagatgacacagagtcctagcagcctgagcgcctcagtgggggatcgggtcaccatcacatgcctggccagcgaagacatatatagcaacctggcctggtaccaacagaagccagggaaaagccctaagctgctgatttactacgccaacagcctcaatgatggagtgccttcacggttttcaggatctggcagcggcactgattttaccctgaccatctctagacttcagccagaggattttgctacctactactgtcaacagaactacgatagcccctacaccttcggacagggcacaaaggtggagatcaagcggacc

SEQ ID NO: 116 - protein, 109 aa, Artificial; identical to SEQ ID NO: 83
  Synthetic Construct

SEQ ID NO: 117 - DNA, 327 bp, Artificial
  Synthetic nucleotide
gacatccagatgacacaaagtccctcttctctgtccgcttccgtgggggacagggtcacaatcacttgccttgccagtgaagacatatattctaatctggcctggtaccagcagaagcctggcaaatctcctcagctcctgatttattacgccaatagtctgaacgatggggtgccaagtcggttttctggcagcggatcaggcacagatttcaccctgactatttcccggcttcagccagaagattttgccacatacttctgtcagcaaaactatgacagcccctacaccttcggccagggcaccaaagtggagatcaagcggacc

SEQ ID NO: 118 - protein, 109 aa, Artificial; identical to SEQ ID NO: 84
  Synthetic Construct

SEQ ID NO: 119 - DNA, 327 bp, Artificial
  Synthetic nucleotide
gacatacagatgactcagtcacccagttcactctctgccagcgtgggggatagggttactatcacatgcctggcctctgaagatatctattccaatctcgcatggtatcagcaaaagcctggaaagagcccacagctcttgatatactacgccaactcactgaacgatggagtgccctcaaggttcagtgggtccggctcaggaaccgatttcaccctgacaatctctagactgcagcctgaggatttcgccacatacttttgtcagcagaactacgacagcccctacacattcggccagggcaccaaactggagatcaagcggacc

SEQ ID NO: 120 - protein, 109 aa, Artificial; identical to SEQ ID NO: 85
  Synthetic Construct

SEQ ID NO: 121 - DNA, 327 bp, Artificial
  Synthetic nucleotide
gatattcagatgacacagagtcctagcagcctgagcgcctcagtgggggatcgggtcaccatcacatgcctggccagcgaagacatatatagcaacctggcctggtaccaacagaagccagggaaaagccctaagctgctgatttactacgccaacagcctcaatgatggagtgccttcacggttttcaggatctggcagcggcactgattttaccctgaccatctctagccttcagccagaggattttgctacctactactgtcaacagaactacgatagcccctacaccttcggacagggcacaaaggtggagatcaagcggacc

SEQ ID NO: 122 - protein, 109 aa, Artificial; identical to SEQ ID NO: 86
  Synthetic Construct

SEQ ID NO: 123 - DNA, 357 bp, Mus musculus
  CDS (1)..(357)
caagtcaagctggaggagtctggggctgagctggtgaagcctggggcctcagtgaagatgtcctgcaaggcttctggctacacatttaccatttacaatatccactgggtaaagcagacgcctagacagggcctggaatggattggaactatttttccagtaaatggtgatacttcctacaatcagagattcaaaggcaaggccacattgactgcagacaaatcctccaacacagcctacatgcagctcagcggcctgacatctgaagactctgcgctctattactgtgcaaggtccactacggctagagcctggtttccttactggggccaagggactctggtcactgtctctgca

SEQ ID NO: 124 - protein, 119 aa, Mus musculus
QVKLEESGAELVKPGASVKMSCKASGYTFTIYNIHWVKQTPRQGLEWIGTIFPVNGDTSYNQRFKGKATLTADKSSNTAYMQLSGLTSEDSALYYCARSTTARAWFPYWGQGTLVTVSA

SEQ ID NO: 125 - DNA, 324 bp, Mus musculus
  CDS (1)..(324)
cacattgtgatcacacagactcaaaaattcatgtccacatcagtaggagacagggtcagcgtcacctgcaaggccagtcagaatgtgggtagtaatgtagcctggtatcaacagaaaccagggcaatctcctaaagtactgatttactcggcatcctaccggtacagtggagtccctgatcgcttcacaggcagtggatctgggacagatttcacactcaccatcagcaatgtgcagtctgaagacttggcagactttttctgtcagcaatataacacctctccgttcaccttcggaggggggaccaagctggaaataaaacgg

SEQ ID NO: 126 - protein, 108 aa, Mus musculus
HIVITQTQKFMSTSVGDRVSVTCKASQNVGSNVAWYQQKPGQSPKVLIYSASYRYSGVPDRFTGSGSGTDFTLTISNVQSEDLADFFCQQYNTSPFTFGGGTKLEIKR

SEQ ID NO: 127 - protein, 5 aa, Mus musculus
IYNIH

SEQ ID NO: 128 - protein, 17 aa, Mus musculus
TIFPVNGDTSYNQRFKG

SEQ ID NO: 129 - protein, 10 aa, Mus musculus
STTARAWFPY

SEQ ID NO: 130 - protein, 11 aa, Mus musculus
KASQNVGSNVA

SEQ ID NO: 131 - protein, 7 aa, Mus musculus
SASYRYS

SEQ ID NO: 132 - protein, 8 aa, Mus musculus
QQYNTSPF